(19) United States
(12) Patent Application Publication
(10) Pub. No.: US 2025/0114408 A1
(43) Pub. Date: Apr. 10, 2025

(54) COMPOSITIONS FOR THE TREATMENT OF SKIN CONDITIONS

(71) Applicant: DERMATA THERAPEUTICS, INC., San Diego, CA (US)

(72) Inventors: Gerald Thomas PROEHL, San Diego, CA (US); Christopher Joseph NARDO, San Diego, CA (US)

(21) Appl. No.: 18/954,103

(22) Filed: Nov. 20, 2024

Related U.S. Application Data
(63) Continuation of application No. 17/251,155, filed on Dec. 10, 2020, now abandoned.
(60) Provisional application No. 62/684,699, filed on Jun. 13, 2018.
(60) Provisional application No. 62/838,801, filed on Apr. 25, 2019.
(60) Provisional application No. 62/788,628, filed on Jan. 4, 2019.

(51) Int. Cl. A61K 35/655; A61K 38/48; A61P 17/00

(52) U.S. Cl. CPC A61K 35/655; A61K 38/4893; A61P 43/00

(57) ABSTRACT

The present disclosure relates to the treatment of a skin condition in a subject, including but not limited to hyperhidrosis, comprising applying to the skin of the subject a first composition derived from one or more sponges, and a second composition comprising one or more botulinum toxins. Also provided are compositions for use in the treatment of a skin condition in a subject, including but not limited to hyperhidrosis, comprising a first composition and a second composition, wherein (a) the first composition comprises Spongilla; and (b) the second composition comprises one or more botulinum toxins. Also provided are kits, including kits for the treatment of a skin condition in a subject including but not limited to hyperhidrosis, comprising a first composition and a second composition, wherein the first composition comprises Spongilla, and the second composition comprises one or more botulinum toxins.

### CROSS REFERENCE

This application claims the priority benefit of U.S. Provisional Application No. 62/684,699, filed Jun. 13, 2018; U.S. Provisional Application No. 62/788,628, filed Jan. 4, 2019, and U.S. Provisional Application No. 62/838,801, filed Apr. 25, 2019, all of which are incorporated by reference herein in their entireties.

### FIELD

The present disclosure relates to the treatment of a skin condition in a subject, including but not limited to hyperhidrosis, comprising applying to the skin of the subject a first composition derived from one or more sponges, and a second composition comprising one or more botulinum toxins. In one aspect, the compositions are derived from one or more sponges. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

The present disclosure relates to the treatment of a skin condition in a subject, including but not limited to hyperhidrosis, comprising applying to the skin of the subject a first composition comprising Spongilla, and a second composition comprising one or more botulinum toxins. The disclosure also relates to a products or kits for the treatment of skin conditions in a subject, including but not limited to hyperhidrosis, comprising a first composition comprising Spongilla, and a second composition comprising one or more botulinum toxins. The disclosure also relates to a first composition comprising Spongilla for use in a method of treating a skin disease or condition in a subject in need thereof, including but not limited to hyperhidrosis, wherein the method comprises administering to the subject an effective amount of the first composition in combination with an effective amount of a second composition, the second composition comprising one or more botulinum toxins.

### BACKGROUND

Skin conditions in subjects, including human subjects, such as acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, and eccrine nevus, can be difficult to treat. In some cases, topical treatment of these skin conditions is successful, but proper usage of topical therapies is often more complex than is the case for oral therapies and adherence may be a particularly significant issue for topical therapies. Poor subject adherence to treatment regimens using topical therapies, development of resistance to medications, and increased costs may contribute to treatment failure. Thus, a method of treating these skin conditions in subjects using topical products having simple usage paradigms, for example that is applied once a week may have the opportunity to exhibit greater treatment success due to improved subject adherence.

A number of skin conditions in subjects have been treated with the topical application of materials derived from naturally occurring sponges, such as Spongilla lacustris. Some materials derived from Spongilla are promoted for the treatment of certain skin conditions, such as acne vulgaris, and as a cosmetic. The Spongilla contains organic and inorganic compounds. The total lipid content is approximately 5% of the biomass of the dried sponge and the protein is composed of spongin or sclerotized collagen. The polysaccharides and N-acetyl-D-glucosamine (NAG) are part of chitin and chitosan that has been reported to be an important component within the skeletal fibers of Spongilla lacustris and detected 750±1.5 μg N-acetyl-D-glucosamine per 1 mg of spicule-free skeleton. Chitin and chitosan are described as a family of linear polysaccharides consisting of varying amounts of α or β (1-4) linked residues of N-acetyl-2 amino-2-deoxy-D-glucose and 2-amino-2-deoxy-D glucose residues. In α-chitin, the chains are arranged in sheets or stacks, the chains in any one sheet having the same direction or ‘sense’. In β-chitin, adjacent sheets along the c axis have the same direction; the sheets are parallel, while in α-chitin adjacent sheets along the c axis have the opposite direction, they are antiparallel. Chitin is deacetylated into chitosan and can be further degraded into N-acetyl-D-glucosamine (NAG) units. Chitosan preparations are classified into native chitosan, chitosan formulations, complexes and derivatives with other substances. Chitosan can be used to prevent or treat wound and burn infections not only because of its intrinsic antimicrobial properties, but also by virtue of its ability to deliver extrinsic antimicrobial agents to wounds and burns. Chitosan is water-insoluble and highly viscous in dilute acidic solutions. Soluble chitosan oligosaccharides were found to be instrumental in suppressing the LPS-induced nuclear factor kappa-light-chain-enhancer of activated B cell (NF-κB)-dependent inflammatory gene expression, and this was associated with reduced nuclear translocation of NF-κB. Chitosan has also been demonstrated to have an antimicrobial effect against P. acnes and S. aureus. Chitosan of differing molecular weight (MW) were tested on antibacterial activity, chitosan of low MW (50-190 kDa), medium MW (190-310 kDa), and high MW (310-375+ kDa). Concentrations of 2.5, 5, 10, and 20 μg/mL were tested against P. acnes with high molecular weight having a greater effect against the gram-positive bacteria P. acnes demonstrated in a clinical study that with acne vulgaris subjects, NAG quickly reduced the number of acne lesions over an 8-week period and was better tolerated by the subjects than 10% benzoyl peroxide.

Hyperhidrosis is a disorder of excessive sweating out of proportion with thermoregulatory requirements. While many subjects may exhibit this excessive sweating in response to specific triggers (e.g., emotional stress), others may exhibit symptoms spontaneously. The diagnosis of hyperhidrosis is based partly on subjective measures that measure how the excessive sweating affects a subject's quality of life. The amount of sweat produced can be measured gravimetrically, though there is no standardized threshold which defines hyperhidrosis. Hyperhidrosis can be categorized as either focal (affecting a specific area such as palms or axillae) or generalized (affecting the entire body). Hyperhidrosis may be secondary to a wide variety of medical conditions, which usually presents as generalized hyperhidrosis. Whereas, primary hyperhidrosis is idiopathic and commonly presents as focal sweating, most frequently on the axillae (51%), soles (30%), palms (24%), and face (10%). Primary focal axillary hyperhidrosis is not uncommon. The prevalence in the US is estimated to be 2.8% of the population, which is comparable to psoriasis. Two-thirds of affected individuals do not seek medical treatment as conventional treatment is generally ineffective and social embarrassment is considerable. Yet, hyperhidrosis has a high burden of disease, primarily due to the effect on subject's quality of life and psychosocial well-being, but also may lead to bacterial and fungal overgrowth. While primary hyperhidrosis is idiopathic, the mechanism is thought to be neurogenic overactivity of eccrine glands in the affected area. The hypothesis for this mechanism is supported by the clinical effect seen with botulinum toxin type A which acts by disrupting sympathetic stimulation to the eccrine glands resulting in considerably reduced axillary sweating from 4 to 12 months. However, the skin penetration, and volume effects from botulinum toxin treatment produce injection site pain. Given the nature of the target tissue, injection site pain is thought to be a major cause for lack of compliance, especially when large numbers of injections have to be placed in sensitive skin areas, such as the axilla. Thus, a topical product with a simple usage paradigm, that would allow for penetration of botulinum toxin past the stratum corneum and into the dermis, may exhibit greater compliance and adoption due to improved tolerability of the treatment.

The inventors of the subject matter disclosed herein have discovered that an important component of materials derived from Spongilla are the siliceous spicules that comprise the skeletal structure of Spongilla. The inventors have discovered the spicules penetrate the stratum corneum of the skin of a subject during application and promote sloughing of the keratinocytes. The inventors of the subject matter disclosed herein have also discovered that the spicules derived from Spongilla are useful in facilitating and permitting certain therapeutic compounds and compositions to penetrate into the skin of subjects to which the spicules are applied, which compounds and compositions would otherwise not be able to penetrate the skin of the subject in order to reach their therapeutic targets and treat certain skin conditions. Among the compounds and compositions that may better penetrate the skin in the presence of materials derived from Spongilla are products containing botulinum toxins.

Products containing botulinum toxins have been demonstrated to be useful for the treatment of a number of medical conditions, including those affecting the skin of subjects. For example, products containing botulinum toxins have been approved for the treatment of subjects suffering from hyperhidrosis (excessive sweating). There have also been reports that the topical application of products containing a botulinum toxin might be useful in the treatment of subjects suffering from acne (see, for example, U.S. Pat. No. 7,226,605). However, the difficulties of using products containing one or more botulinum toxins for the topical treatment of skin conditions in subjects is well known. One difficulty in the topical use of botulinum toxin-containing products is the recognition that the endogenous non-toxin proteins in a botulinum toxin complex obtained from Clostridium botulinum bacteria (viz., the non-toxic hemagglutinin and non-hemagglutinin proteins) decrease the ability of the toxin to diffuse through the skin epithelium. These effects can be further exacerbated when an exogenous stabilizer, such as albumin, binds to botulinum toxin during conventional manufacturing processes. Therefore, the application of compositions comprising Spongilla, for example in the form of a powder, to the skin of a subject will help facilitate the penetration of topically-applied products containing one or more botulinum toxins into the skin of the subject, leading to the use of new treatment regimens for sometimes difficult to treat skin conditions.

### SUMMARY OF THE INVENTION

In one aspect is provided a method of treating a skin condition in a subject, comprising applying to the skin of the subject a first composition comprising Spongilla, and a second composition comprising one or more botulinum toxins.

In one aspect is provided a method of treating a skin condition in a subject, comprising applying to the skin of the subject a first composition comprising one or more sponges, and a second composition comprising one or more botulinum toxins. In one aspect, the compositions are derived from one or more sponges. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

In one aspect is provided a method of treating a skin condition in a subject, comprising applying to the skin of the subject a first composition comprising one or more sponges, and a second composition comprising one or more botulinum toxins, wherein (a) the second composition is comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G; and (b) the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrophic acne scars, and melasma. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

In one aspect is provided a method of treating a skin condition in a subject, comprising applying to the skin of the subject a first composition and a second composition, wherein (a) the first composition comprises one or more sponges; (b) the second composition is comprises one or more botulinum toxin type selected from botulinum toxin type A, botulinum toxin type B, and botulinum toxin type E; and (c) the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

In another aspect is provided a kit, comprising a first composition and a second composition, wherein (a) the first composition comprises one or more sponges; and (b) the second composition comprises one or more botulinum toxins. In another aspect is provided a kit comprising a first composition and a second composition, wherein (a) the first composition comprises a Spongilla; and (b) the second composition comprises one or more botulinum toxins, wherein the kit is used for the treatment of a skin condition in a subject. In another aspect is provided a kit comprising a first composition and a second composition, wherein (a) the first composition comprises a Spongilla; and (b) the second composition comprises one or more botulinum toxins, wherein the kit is for use in the treatment of a skin condition in a subject. In another aspect is provided a kit as described herein, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

In another aspect is provided a composition comprising a first composition and a second composition for use as a medicament, wherein the (a) the first composition comprises one or more sponges; and (b) the second composition one or more botulinum toxins. In another aspect is provided a composition comprising a first composition and a second composition for use as a medicament, wherein the (a) the first composition comprises a Spongilla lacustris; and (b) the second composition one or more botulinum toxins. In another aspect is provided such compositions for use as a medicament for the treatment of a skin condition in a subject. In another aspect is such combinations for use as a medicament for the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

In another aspect is provided a composition comprising a first composition and a second composition for use in the treatment of a skin condition in a subject, wherein (a) the first composition comprises one or more sponges; and (b) the second composition one or more botulinum toxins. In another aspect is provided a combination comprising a first composition and a second composition for use in the treatment of a skin condition in a subject, wherein (a) the first composition comprising a sponge; and (b) the second composition one or more botulinum toxins. In another aspect is such compositions for use in the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

In another aspect is provided a composition for the manufacture of a medicament for the treatment of a skin condition in a subject, wherein the composition comprises a first composition and a second composition wherein (a) the first composition comprises one or more sponges; and (b) the second composition one or more botulinum toxins. In another aspect is provided a composition for the manufacture of a medicament for the treatment of a skin condition in a subject, wherein the composition comprises a first composition and a second composition wherein (a) the first composition comprising a Spongilla lacustris; and (b) the second composition one or more botulinum toxins. In another aspect is provided a composition for the manufacture of a medicament for the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

In one aspect is provided a method of treating a skin condition in a subject, comprising applying to the skin of the subject a first composition comprising Spongilla, and a second composition comprising one or more botulinum toxins, wherein (a) the second composition is comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G; and (b) the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrophic acne scars, and melasma.

In one aspect is provided a method of treating a skin condition in a subject, comprising applying to the skin of the subject a first composition and a second composition, wherein (a) the first composition comprises Spongilla powder; (b) the second composition is comprises one or more botulinum toxin type selected from botulinum toxin type A, botulinum toxin type B, and botulinum toxin type E; and (c) the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma.

In another aspect is provided a kit, comprising a first composition and a second composition, wherein (a) the first composition comprises a Spongilla; and (b) the second composition comprises one or more botulinum toxins. In another aspect is provided a kit comprising a first composition and a second composition, wherein (a) the first composition comprises a Spongilla; and (b) the second composition comprises one or more botulinum toxins, wherein the kit is used for the treatment of a skin condition in a subject. In another aspect is provided a kit comprising a first composition and a second composition, wherein (a) the first composition comprises a Spongilla; and (b) the second composition comprises one or more botulinum toxins, wherein the kit is for use in the treatment of a skin condition in a subject. In another aspect is provided a kit as described herein, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma.

In another aspect is provided a composition comprising a first composition and a second composition for use as a medicament, wherein the (a) the first composition comprises a Spongilla; and (b) the second composition one or more botulinum toxins. In another aspect is provided a composition comprising a first composition and a second composition for use as a medicament, wherein the (a) the first composition comprises a Spongilla lacustris; and (b) the second composition one or more botulinum toxins. In another aspect is provided such compositions for use as a medicament for the treatment of a skin condition in a subject. In another aspect is such combinations for use as a medicament for the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma.

In another aspect is provided a composition comprising a first composition and a second composition for use in the treatment of a skin condition in a subject, wherein (a) the first composition comprising a Spongilla; and (b) the second composition one or more botulinum toxins. In another aspect is provided a combination comprising a first composition and a second composition for use in the treatment of a skin condition in a subject, wherein (a) the first composition comprising a Spongilla lacustris; and (b) the second composition one or more botulinum toxins. In another aspect is such compositions for use in the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma.

In another aspect is provided a composition for the manufacture of a medicament for the treatment of a skin condition in a subject, wherein the composition comprises a first composition and a second composition wherein (a) the first composition comprising a Spongilla; and (b) the second composition one or more botulinum toxins. In another aspect is provided a composition for the manufacture of a medicament for the treatment of a skin condition in a subject, wherein the composition comprises a first composition and a second composition wherein (a) the first composition comprising a Spongilla lacustris; and (b) the second composition one or more botulinum toxins. In another aspect is provided a composition for the manufacture of a medicament for the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma.

### DETAILED DESCRIPTION

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

As used herein, the term “about” means either within plus or minus 10% of the provided value, or rounded to the nearest significant figure, in all cases inclusive of the provided value. Where ranges are provided, they are inclusive of the boundary values.

As used herein, the terms “applied,” “applying,” “administration,” “administering,” and “used” means the delivery of a composition disclosed herein to a subject, in particular to the skin of the subject, by an administration route including, but not limited to, intraperitoneal, subcutaneous, intramuscular, topically, or any combinations thereof. In some embodiments disclosed herein, the compositions disclosed herein are administered to the subject, in particular to the skin of the subject, by topical administration.

As used herein, the term “aspect ratio” means with respect to the particles of Spongilla described herein the ratio between the average length of the particles to the average diameter of the particles.

As used herein, the term “botulinum toxin” means a protein produced by the bacterium Clostridium botulinum and related species. As used herein, the term “botulinum toxin type A” means a protein known to those of ordinary skill in the art as the protein also referred to as “BoNT/A” or “botA,” and having representative UniProt reference numbers BXA1_CLOBH (strain Hall), BXA1_CLOBO, BXA2_CLOBO, or variants thereof. As used herein, the term “botulinum toxin type B” means a protein known to those of ordinary skill in the art as the protein also referred to as “BoNT/B” or “botB,” and having representative UniProt reference number BXB_CLOBO, or variants thereof. As used herein, the term “botulinum toxin type C” means a protein known to those of ordinary skill in the art as the protein also referred to as “BoNT/C1” and having representative UniProt reference numbers BXC1_CLOBO, or variants thereof. As used herein, the term “botulinum toxin type C₂” means a protein known to those of ordinary skill in the art as the protein referred to as botulinum toxin type C₂. As used herein, the term “botulinum toxin type D” means a protein known to those of ordinary skill in the art as the protein also referred to as “BoNT/D” or “botD,” and having representative UniProt reference number BXD_CLOBO, or variants thereof. As used herein, the term “botulinum toxin type E” means a protein known to those of ordinary skill in the art as the protein also referred to as “BoNT/E” and having representative UniProt reference number BXE_CLOBO, or variants thereof. As used herein, the term “botulinum toxin type F” means a protein known to those of ordinary skill in the art as the protein also referred to as “BoNT/F” or “botF,” and having representative UniProt reference number BXF_CLOBO, or variants thereof. As used herein, the term “botulinum toxin type G” means a protein known to those of ordinary skill in the art as the protein also referred to as “BoNT/E” or “botG,” and having representative UniProt reference number BXG_CLOBO, or variants thereof. As used herein, the term “variants” means proteins having 70%, 75%, 80%, 85%, 90%, 91%, 92%, 93%, 94%, 95%, 96%, 97%, 98% or 99% or any percentage in between homology.

As used herein, the terms “combination” and “in combination with” mean the application, use, or administration of one or more of the compositions disclosed herein, sequentially or simultaneously. It includes dosing simultaneously, or within minutes or hours of each other, or on the same day, or on alternating days, or using the compositions disclosed herein on a daily basis, or multiple days per week, or weekly basis, for example, while administering another composition on the same day or alternating days or weeks or on a periodic basis during a time simultaneous therewith or concurrent therewith, or at least a part of the time during which the composition disclosed herein is applied, used or administered. For example, one or more of the compositions disclosed herein, could be applied, used, or administered to a subject every day or several days a week while the additional composition is applied, used or dosed on alternating days or alternating weeks or other periods of time, such as every 1, 2, 3, 4, 5, 6, 7, 8, 9, 10, 11, 12, 13, 14 or more days.

The term “abobotulinumtoxinA” as used herein means the botulinum toxin type A product approved by the FDA under BLA No. 125274.

The term “Chalinidea” as used herein means one or more sponges of the family Chalinidea.

The term “daxibotulinumtoxinA” means daxibotulinumtoxinA 150 kiloDalton (kDa) purified botulinum toxin type A complex currently in development by Revance Therapeutics, Inc.

The term “Demospongiae” means one or more sponges of the class Demospongiae.

The term “EB-001A” as used herein means the botulinum toxin type E product being developed by Bonti, Inc. of Orange County, California.

The term “EB-001T” as used herein means the botulinum toxin type E product being developed by Bonti, Inc. of Orange County, California.

The term “Halciona” as used herein means one or more sponges from the genus Halciona.

The term “Haplosclerida” as used herein means one or more sponges of the order Haplosclerida.

The term “incobotulinumtoxinA” as used herein, means the botulinum toxin type A product approved by the FDA under BLA No. 125360.

The term “onabotulinumtoxinA” as used herein means the botulinum toxin type A product approved by the United States Food and Drug Administration (“FDA”) under Biologics License Application (“BLA”) No. 103000.

The term “rimabotulintoxin B” as used herein means the botulinum toxin type B product approved by the FDA under BLA No. 103846.

The term “Porifera” as used herein means one or more sponge members of the phylum Porifera.

The term “prabotulinumtoxinA” means prabotulinumtoxinA 900 kiloDalton (kDa) purified botulinum toxin type A complex currently in development by Evolus, Inc. and Daewoong Pharmaceutical Co. Ltd.

The term “Spongilla” as used herein means a genus of freshwater sponges in the family Spongillidae, including, but not limited to, Spongilla lacustris, S. fragilis Leidy, and Ephydatia fluviatilis. The term “Spongilla lacustris” as used herein means a species of sponge of the freshwater sponge family Spongillidae.

The terms “composition comprising one or more sponges,” “powders comprising one or more sponges, “materials comprising one or more sponges,” “sponge in the form of a powder,” and the like, as used herein, mean materials derived from one or more sponges that is harvested and processed and may include all the various components of the sponge following harvest, including all organic and/or inorganic compounds and materials that are part of the naturally-occurring sponge, or any sponges that are specially grown or adapted for use in the disclosed compositions, methods and/or kits, or may include only a portion of the organic and/or inorganic compounds and materials that are part of the naturally-occurring sponge. In one aspect is provided any of the methods or kits disclosed herein, wherein the sponge materials comprise all or substantially all the organic and inorganic materials derived from the naturally occurring sponge. In another aspect is provided any of the methods or kits disclosed herein, wherein the sponge materials comprise (a) only the spicules and any materials that are naturally associated with the spicules, or (b) substantially purified spicules and any materials that are naturally associated with the spicules, or (c) purified spicules and any materials that are naturally associated with the spicules that are a component part of naturally-occurring sponge. In another aspect is provided any of the methods or kits disclosed herein, wherein the sponge materials comprise only the spicules and any materials that are naturally associated with the spicules. In another aspect is provided any of the methods or kits disclosed herein, wherein the sponge materials comprise substantially purified spicules and any materials that are naturally associated with the spicules. In another aspect is provided any of the methods or kits disclosed herein, wherein the sponge materials comprise purified spicules and any materials that are naturally associated with the spicules that are a component part of naturally occurring sponge. These terms may be used herein in relation to materials derived from the phylum Porifera. In another aspect, the materials are derived from sponges of the class Demospongiae. In another aspect, the materials are derived from sponges of the order Spongdilla. In another aspect, the materials are derived from sponges of the family Spongillidae. In another aspect, the materials are derived from sponges of the genus Spongilla. In another aspect, the materials are derived from sponges of the species Spongilla lacustris. In another aspect, the materials are derived from sponges of the order Haplosclerida. In another aspect, the materials are derived from sponges of the family Chalinidea. In another aspect, the materials are derived from sponges of the genus Halciona.

The terms “composition comprising Spongilla,” “powders comprising Spongilla, “materials comprising Spongilla, “Spongilla in the form of a powder,” and the like, as used herein, mean materials comprising Spongilla derived from raw Spongilla that is harvested and processed and may include all the various components of the Spongilla following harvest, including all organic and/or inorganic compounds and materials that are part of the naturally-occurring Spongilla, or may include only a portion of the organic and/or inorganic compounds and materials that are part of the naturally-occurring Spongilla. In one aspect is provided any of the methods or kits disclosed herein, wherein the Spongilla materials comprise all or substantially all the organic and inorganic materials derived from the naturally occurring Spongilla. In another aspect is provided any of the methods or kits disclosed herein, wherein the Spongilla materials comprise (a) only the spicules and any materials that are naturally associated with the spicules, or (b) substantially purified spicules and any materials that are naturally associated with the spicules, or (c) purified spicules and any materials that are naturally associated with the spicules that are a component part of naturally-occurring Spongilla. In another aspect is provided any of the methods or kits disclosed herein, wherein the Spongilla materials comprise only the spicules and any materials that are naturally associated with the spicules. In another aspect is provided any of the methods or kits disclosed herein, wherein the Spongilla materials comprise substantially purified spicules and any materials that are naturally associated with the spicules. In another aspect is provided any of the methods or kits disclosed herein, wherein the Spongilla materials comprise purified spicules and any materials that are naturally associated with the spicules that are a component part of naturally occurring Spongilla.

As used herein, the term “subject” has the meaning given to the term by one having ordinary skill in the art and may mean a mammal, including a human, a dog, a cat, cattle, or a pig. In one embodiment the subject is a human. In one embodiment, the subject is a dog. In one embodiment, the subject is a cat. In one embodiment, the subject is cattle. In one embodiment, the subject is a pig.

As used herein, the term “therapeutically effective amount” means that amount of the composition or combination of compositions being applied, used or administered to a subject that will treat, relieve, or prevent to some extent one or more of the symptoms of the disorder being treated.

Spongilla, including Spongilla lacustris, and powders prepared from Spongilla that are utilized in the methods disclosed herein may be obtained, processed and characterized by methods known to those having ordinary skill in the art. For example, U.S. Pat. No. 7,604,821 describes the harvest, processing and characterization of several species of Spongilla, including Spongilla lacustris. The disclosure of U.S. Pat. No. 7,604,821 is incorporated herein by reference in its entirety. Sponge materials may be collected using methods commonly known to those skilled in the art of marine biology. For example, sponges can be collected manually using basic under water diving techniques, or in deeper waters larger colonies are harvested using the Agassiz trawl (AGT) or epibenthic sledge (EBS). Under certain environmental conditions, Spongilla colonies occur in a thin crust-like carpet several meters across and must be collected manually, with fork-like tools, and nets. The collected sponge mass is dried, cleaned of gross contamination, such as shells, stems, plants, rocks and other impurities, and is then washed to remove dirt, sand, silt and soluble impurities. The cleaned sponge mass is weighed and dried using methods known to those of ordinary skill in the art, such as air drying and the use of dryers that are used to dehydrate foods and pharmaceuticals. The sponge mass is dried until residual moisture content is less than a desired value as further disclosed herein. Residual moisture measurements can be performed using methods commonly known in the arts of food sciences, analytical chemistry or the pharmaceutical sciences. For example, 10 grams of dried material may be placed on a tared weighing boat and then weighed. The weighed material is then exposed to a heat source such as a drying oven or heat lamp operated at an appropriate temperature, the sample is then cooled in a desiccated chamber and re-weighed. Residual moisture is calculated as the percent difference between the sample weight before drying and the weight after cooling. Following drying, the sponge materials may be packaged in sealed containers, which optionally protect the materials from light, moisture and oxygen. The materials may then be further tested for the presence of pathogens, coliform organisms and organisms that represent a bioburden. The materials may be further heated or irradiated, as disclosed herein, to reduce any pathogens, coliform organisms or other organisms that represent a bioburden. The materials may then be further processed using methods known to those having ordinary skill in the art to provide a powder comprising particles having a desired size. For example, the sponge materials may be ground, and the resulting materials passed through one or more sieves of a defined size to provide a resulting material comprising particles having a uniform, or substantially uniform size. After final processing and sizing processes are completed, the dried sponge material may be packaged in airtight moisture-proof containers and stored at an appropriate temperature, such as at about room or ambient temperature.

Materials derived from sponges other than Spongilla lacustris may be prepared according to the methods described above and those known to those of ordinary skill in the art. In particular, these methods may be applied with respect to sponges of the phylum Porifera. In another aspect, these methods may be applied with respect to sponges of the class Demospongiae. In another aspect, these methods may be applied with respect to sponges of the order Spongdilla. In another aspect, these methods may be applied with respect to sponges of the family Spongillidae. In another aspect, these methods may be applied with respect to sponges of the genus Spongilla. In another aspect, these methods may be applied with respect to sponges of the species Spongilla lacustris. In another aspect, these methods may be applied with respect to sponges of the order Haplosclerida. In another aspect, these methods may be applied with respect to sponges of the family Chalinidea. In another aspect, these methods may be applied with respect to sponges of the genus Halciona.

In one aspect are provided methods of treating a skin condition in a subject, comprising applying to the skin of the subject in need thereof an effective amount of a first composition comprising Spongilla, and an effective amount of a second composition comprising one or more botulinum toxins, wherein:
(a) the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G; and
(b) the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another aspect is provided any of the methods, wherein the skin condition in the subject is hyperhidrosis, acne vulgaris and rosacea. In one aspect is provided any of the methods, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided any of the methods disclosed herein, wherein the one or more botulinum toxin type is selected from botulinum toxin type A, botulinum toxin type B, and botulinum toxin type E. In another aspect is provided any of the methods disclosed herein, wherein the one or more botulinum toxin type is botulinum toxin type A. In another aspect is provided any of the methods disclosed herein, wherein the botulinum toxin type A is selected from onabotulinumtoxinA, abobotulinumtoxinA, incobotulinumtoxinA, prabotulinumtoxinA, and daxibotulinumtoxinA. In another aspect is provided any of the methods disclosed herein, wherein the botulinum toxin type A is onabotulinumtoxinA. In another aspect is provided any of the methods disclosed herein, wherein the botulinum toxin type A is abobotulinumtoxinA. In another aspect is provided any of the methods disclosed herein, wherein the botulinum toxin type A is incobotulinumtoxinA. In another aspect is provided any of the methods disclosed herein, wherein the botulinum toxin type A is prabotulinumtoxinA. In another aspect is provided any of the methods disclosed herein, wherein the one or more botulinum toxin type is botulinum toxin type B. In another aspect is provided any of the methods disclosed herein, wherein the one or more botulinum toxin type B is rimabotulinumtoxinB. In another aspect is provided any of the methods disclosed herein, wherein the one or more botulinum toxin type is botulinum toxin type C1. In another aspect is provided any of the methods disclosed herein, wherein the one or more botulinum toxin type is botulinum toxin type C2. In another aspect is provided any of the methods disclosed herein, wherein the one or more botulinum toxin type is botulinum toxin type D. In another aspect is provided any of the methods disclosed herein, wherein the one or more botulinum toxin type is botulinum toxin type E. In another aspect is provided any of the methods disclosed herein, wherein the one or more botulinum toxin type E is EB-001A or EB-001T. In another aspect is provided any of the methods disclosed herein, wherein the one or more botulinum toxin type E is EB-001A. In another aspect is provided any of the methods disclosed herein, wherein the one or more botulinum toxin type E is EB-001T. In another aspect is provided any of the methods disclosed herein, wherein the one or more botulinum toxin type is botulinum toxin type F. In another aspect is provided any of the methods disclosed herein, wherein the one or more botulinum toxin type is botulinum toxin type G. In another aspect is provided any of the methods disclosed herein, wherein the skin condition in the subject is selected from acne vulgaris, acne rosacea type 1, acne rosacea type 2, psoriasis, and hyperhidrosis. In another aspect is provided any of the methods disclosed herein, wherein the skin condition in the subject is acne vulgaris. In another aspect is provided any of the methods disclosed herein, wherein the skin condition in the subject is acne rosacea type 1. In another aspect is provided any of the methods disclosed herein, wherein the skin condition in the subject is acne rosacea type 2. In another aspect is provided any of the methods disclosed herein, wherein the skin condition in the subject is psoriasis. In another aspect is provided any of the methods disclosed herein, wherein the skin condition in the subject is hyperhidrosis. In another aspect is provided any of the methods, wherein the first composition comprising Spongilla is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In another aspect is provided any of the methods disclosed herein, wherein the first composition comprising Spongilla is applied to the skin of the subject once per week for 2 weeks. In another aspect is provided any of the methods disclosed herein, wherein the first composition comprising Spongilla is applied to the skin of the subject once per week for 3 weeks. In another aspect is provided any of the methods disclosed herein, wherein the first composition comprising Spongilla is applied to the skin of the subject once per week for 4 weeks. In another aspect is provided any of the methods disclosed herein, wherein the first composition comprising Spongilla is applied to the skin of the subject once per week for 5 weeks. In another aspect is provided any of the methods disclosed herein, wherein the first composition comprising Spongilla is applied to the skin of the subject once per week for 6 weeks. In another aspect is provided any of the methods disclosed herein, wherein the first composition comprising Spongilla is applied to the skin of the subject once per week for 7 weeks. In another aspect is provided any of the methods disclosed herein, wherein the first composition comprising Spongilla is applied to the skin of the subject once per week for 8 weeks.

In one aspect are provided methods of hyperhidrosis in a subject, comprising applying to the skin of the subject in need thereof an effective amount of a first composition comprising Spongilla, and an effective amount of a second composition comprising one or more botulinum toxins, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In one aspect is provided any of the methods, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided any of the methods disclosed herein, wherein the one or more botulinum toxin type is selected from botulinum toxin type A, botulinum toxin type B, and botulinum toxin type E. In another aspect is provided any of the methods disclosed herein, wherein the one or more botulinum toxin type is botulinum toxin type A. In another aspect is provided any of the methods disclosed herein, wherein the botulinum toxin type A is selected from onabotulinumtoxinA, abobotulinumtoxinA, incobotulinumtoxinA, prabotulinumtoxinA, and daxibotulinumtoxinA. In another aspect is provided any of the methods disclosed herein, wherein the botulinum toxin type A is onabotulinumtoxinA. In another aspect is provided any of the methods disclosed herein, wherein the botulinum toxin type A is abobotulinumtoxinA. In another aspect is provided any of the methods disclosed herein, wherein the botulinum toxin type A is incobotulinumtoxinA. In another aspect is provided any of the methods disclosed herein, wherein the botulinum toxin type A is prabotulinumtoxinA. In another aspect is provided any of the methods disclosed herein, wherein the one or more botulinum toxin type is botulinum toxin type B. In another aspect is provided any of the methods disclosed herein, wherein the one or more botulinum toxin type B is rimabotulinumtoxinB. In another aspect is provided any of the methods disclosed herein, wherein the one or more botulinum toxin type is botulinum toxin type C1. In another aspect is provided any of the methods disclosed herein, wherein the one or more botulinum toxin type is botulinum toxin type C2. In another aspect is provided any of the methods disclosed herein, wherein the one or more botulinum toxin type is botulinum toxin type D. In another aspect is provided any of the methods disclosed herein, wherein the one or more botulinum toxin type is botulinum toxin type E. In another aspect is provided any of the methods disclosed herein, wherein the one or more botulinum toxin type E is EB-001A or EB-001T. In another aspect is provided any of the methods disclosed herein, wherein the one or more botulinum toxin type E is EB-001A. In another aspect is provided any of the methods disclosed herein, wherein the one or more botulinum toxin type E is EB-001T. In another aspect is provided any of the methods disclosed herein, wherein the one or more botulinum toxin type is botulinum toxin type F. In another aspect is provided any of the methods disclosed herein, wherein the one or more botulinum toxin type is botulinum toxin type G. In another aspect is provided any of the methods, wherein the first composition comprising Spongilla is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In another aspect is provided any of the methods disclosed herein, wherein the first composition comprising Spongilla is applied to the skin of the subject once per week for 2 weeks. In another aspect is provided any of the methods disclosed herein, wherein the first composition comprising Spongilla is applied to the skin of the subject once per week for 3 weeks. In another aspect is provided any of the methods disclosed herein, wherein the first composition comprising Spongilla is applied to the skin of the subject once per week for 4 weeks. In another aspect is provided any of the methods disclosed herein, wherein the first composition comprising Spongilla is applied to the skin of the subject once per week for 5 weeks. In another aspect is provided any of the methods disclosed herein, wherein the first composition comprising Spongilla is applied to the skin of the subject once per week for 6 weeks. In another aspect is provided any of the methods disclosed herein, wherein the first composition comprising Spongilla is applied to the skin of the subject once per week for 7 weeks. In another aspect is provided any of the methods disclosed herein, wherein the first composition comprising Spongilla is applied to the skin of the subject once per week for 8 weeks.

In one aspect are provided methods of hyperhidrosis in a subject, comprising applying to the skin of the subject in need thereof an effective amount of a first composition comprising Spongilla, and an effective amount of a second composition comprising botulinum toxin type A. In another aspect is provided any of the methods disclosed herein, wherein the botulinum toxin type A is selected from onabotulinumtoxinA, abobotulinumtoxinA, incobotulinumtoxinA, prabotulinumtoxinA, and daxibotulinumtoxinA. In another aspect is provided any of the methods disclosed herein, wherein the botulinum toxin type A is onabotulinumtoxinA. In another aspect is provided any of the methods disclosed herein, wherein the botulinum toxin type A is abobotulinumtoxinA. In another aspect is provided any of the methods disclosed herein, wherein the botulinum toxin type A is incobotulinumtoxinA. In another aspect is provided any of the methods disclosed herein, wherein the botulinum toxin type A is prabotulinumtoxinA. In another aspect is provided any of the methods, wherein the first composition comprising Spongilla is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In another aspect is provided any of the methods disclosed herein, wherein the first composition comprising Spongilla is applied to the skin of the subject once per week for 2 weeks. In another aspect is provided any of the methods disclosed herein, wherein the first composition comprising Spongilla is applied to the skin of the subject once per week for 3 weeks. In another aspect is provided any of the methods disclosed herein, wherein the first composition comprising Spongilla is applied to the skin of the subject once per week for 4 weeks. In another aspect is provided any of the methods disclosed herein, wherein the first composition comprising Spongilla is applied to the skin of the subject once per week for 5 weeks. In another aspect is provided any of the methods disclosed herein, wherein the first composition comprising Spongilla is applied to the skin of the subject once per week for 6 weeks. In another aspect is provided any of the methods disclosed herein, wherein the first composition comprising Spongilla is applied to the skin of the subject once per week for 7 weeks. In another aspect is provided any of the methods disclosed herein, wherein the first composition comprising Spongilla is applied to the skin of the subject once per week for 8 weeks.

A first composition comprising Spongilla for use in a method of treating a skin disease or condition in a subject in need thereof, wherein the method comprises administering to the subject an effective amount of the first composition in combination with an effective amount of a second composition, the second composition comprising one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G, and wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another aspect is provided such a first composition for use, wherein the skin condition or condition in the subject is hyperhidrosis, acne vulgaris and rosacea. In one aspect is provided such a first composition for use, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In one aspect is provided such a first composition for use wherein the one or more botulinum toxin type is selected from botulinum toxin type A, botulinum toxin type B, and botulinum toxin type E. In one aspect is provided such a first composition for use wherein the one or more botulinum toxin type is botulinum toxin type A. In one aspect is provided such a first composition for use, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a first composition for use wherein the botulinum toxin type A is onabotulinumtoxinA. In one aspect is provided such a first composition for use wherein the botulinum toxin type A is abobotulinumtoxinA. In one aspect is provided such a first composition for use, wherein the botulinum toxin type A is incobotulinumtoxinA. In one aspect is provided such a first composition for use, wherein the botulinum toxin type A is prabotulinumtoxinA. In one aspect is provided such a first composition for use, wherein the one or more botulinum toxin type is botulinum toxin type B. In another aspect is provided any of the methods disclosed herein, wherein the one or more botulinum toxin type B is rimabotulinumtoxinB. In one aspect is provided such a first composition for use, wherein the one or more botulinum toxin type is botulinum toxin type C1. In one aspect is provided such a first composition for use, wherein the one or more botulinum toxin type is botulinum toxin type C2. In one aspect is provided such a first composition for use, wherein the one or more botulinum toxin type is botulinum toxin type D In one aspect is provided such a first composition for use, wherein the one or more botulinum toxin type is botulinum toxin type E. In one aspect is provided such a first composition for use, wherein the one or more botulinum toxin type E is EB-001A or EB-001T. In one aspect is provided such a first composition for use, wherein the one or more botulinum toxin type E is EB-001A. In one aspect is provided such a first composition for use, wherein the one or more botulinum toxin type E is EB-001T. In one aspect is provided such a first composition for use, wherein the one or more botulinum toxin type is botulinum toxin type F. In one aspect is provided such a first composition for use, wherein the one or more botulinum toxin type is botulinum toxin type G. In one aspect is provided such a first composition for use, wherein the skin condition in the subject is selected from acne vulgaris, acne rosacea type 1, acne rosacea type 2, psoriasis, and hyperhidrosis. In one aspect is provided such a first composition for use, wherein the skin condition in the subject is acne vulgaris. In one aspect is provided such a first composition for use, wherein the skin condition in the subject is acne rosacea type 1. In one aspect is provided such a first composition for use, wherein the skin condition in the subject is acne rosacea type 2 In one aspect is provided such a first composition for use, wherein the skin condition in the subject is psoriasis. In one aspect is provided such a first composition for use, wherein the skin condition in the subject is hyperhidrosis. In one aspect is provided such a first composition for use, wherein the first composition comprising Spongilla is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In one aspect is provided such a first composition for use, wherein the first composition comprising Spongilla is applied to the skin of the subject once per week for 2 weeks. In one aspect is provided such a first composition for use, wherein the first composition comprising Spongilla is applied to the skin of the subject once per week for 3 weeks. In one aspect is provided such a first composition for use, wherein the first composition comprising Spongilla is applied to the skin of the subject once per week for 4 weeks. In one aspect is provided such a first composition for use, wherein the first composition comprising Spongilla is applied to the skin of the subject once per week for 5 weeks. In one aspect is provided such a first composition for use, wherein the first composition comprising Spongilla is applied to the skin of the subject once per week for 6 weeks. In one aspect is provided such a first composition for use, wherein the first composition comprising Spongilla is applied to the skin of the subject once per week for 7 weeks. In one aspect is provided such a first composition for use, wherein the first composition comprising Spongilla is applied to the skin of the subject once per week for 8 weeks.

A first composition comprising Spongilla for use in a method of treating hyperhidrosis in a subject in need thereof, wherein the method comprises administering to the subject an effective amount of the first composition in combination with an effective amount of a second composition, the second composition comprising botulinum toxin type A. In one aspect is provided such a first composition for use, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a first composition for use wherein the botulinum toxin type A is onabotulinumtoxinA. In one aspect is provided such a first composition for use wherein the botulinum toxin type A is abobotulinumtoxinA. In one aspect is provided such a first composition for use, wherein the botulinum toxin type A is incobotulinumtoxinA. In one aspect is provided such a first composition for use, wherein the botulinum toxin type A is prabotulinumtoxinA. In one aspect is provided such a first composition for use, wherein the first composition comprising Spongilla is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In one aspect is provided such a first composition for use, wherein the first composition comprising Spongilla is applied to the skin of the subject once per week for 2 weeks. In one aspect is provided such a first composition for use, wherein the first composition comprising Spongilla is applied to the skin of the subject once per week for 3 weeks. In one aspect is provided such a first composition for use, wherein the first composition comprising Spongilla is applied to the skin of the subject once per week for 4 weeks. In one aspect is provided such a first composition for use, wherein the first composition comprising Spongilla is applied to the skin of the subject once per week for 5 weeks. In one aspect is provided such a first composition for use, wherein the first composition comprising Spongilla is applied to the skin of the subject once per week for 6 weeks. In one aspect is provided such a first composition for use, wherein the first composition comprising Spongilla is applied to the skin of the subject once per week for 7 weeks. In one aspect is provided such a first composition for use, wherein the first composition comprising Spongilla is applied to the skin of the subject once per week for 8 weeks.

A first composition comprising Spongilla for use in a method of treating hyperhidrosis in a subject in need thereof, wherein the method comprises administering to the subject an effective amount of the first composition in combination with an effective amount of a second composition, the second composition comprising onabotulinumtoxinA. In one aspect is provided such a first composition for use, wherein the first composition comprising Spongilla is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In one aspect is provided such a first composition for use, wherein the first composition comprising Spongilla is applied to the skin of the subject once per week for 2 weeks. In one aspect is provided such a first composition for use, wherein the first composition comprising Spongilla is applied to the skin of the subject once per week for 3 weeks. In one aspect is provided such a first composition for use, wherein the first composition comprising Spongilla is applied to the skin of the subject once per week for 4 weeks. In one aspect is provided such a first composition for use, wherein the first composition comprising Spongilla is applied to the skin of the subject once per week for 5 weeks. In one aspect is provided such a first composition for use, wherein the first composition comprising Spongilla is applied to the skin of the subject once per week for 6 weeks. In one aspect is provided such a first composition for use, wherein the first composition comprising Spongilla is applied to the skin of the subject once per week for 7 weeks. In one aspect is provided such a first composition for use, wherein the first composition comprising Spongilla is applied to the skin of the subject once per week for 8 weeks.

A composition comprising Spongilla and one or more botulinum toxins for the treatment of a skin condition or disease in a subject in need thereof, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another aspect is provided such a first composition for use, wherein the skin condition or condition in the subject is hyperhidrosis, acne vulgaris and rosacea, and wherein the botulinum toxin is selected from botulinum toxin type A, botulinum toxin type B, botulinum toxin type C1, botulinum toxin type C2, botulinum toxin type D, botulinum toxin type E, botulinum toxin type F and botulinum toxin type G, and wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another aspect is provided such a composition for use, wherein the skin condition or condition in the subject is hyperhidrosis, acne vulgaris and rosacea. In one aspect is provided such a composition for use, wherein botulinum toxin is selected from botulinum toxin type A, botulinum toxin type B, botulinum toxin type C1, botulinum toxin type C2, botulinum toxin type D, botulinum toxin type E, botulinum toxin type F and botulinum toxin type G. In one aspect is provided such a composition for use wherein the one or more botulinum toxin type is selected from botulinum toxin type A, botulinum toxin type B, and botulinum toxin type E. In one aspect is provided such a composition for use wherein the one or more botulinum toxin type is botulinum toxin type A. In one aspect is provided such a composition for use, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a composition for use wherein the botulinum toxin type A is onabotulinumtoxinA. In one aspect is provided such a composition for use wherein the botulinum toxin type A is abobotulinumtoxinA. In one aspect is provided such a composition for use, wherein the botulinum toxin type A is incobotulinumtoxinA. In one aspect is provided such a composition for use, wherein the botulinum toxin type A is prabotulinumtoxinA. In one aspect is provided such a composition for use, wherein the one or more botulinum toxin type is botulinum toxin type B. In another aspect is provided any of the methods disclosed herein, wherein the one or more botulinum toxin type B is rimabotulinumtoxinB. In one aspect is provided such a composition for use, wherein the one or more botulinum toxin type is botulinum toxin type C1. In one aspect is provided such a composition for use, wherein the one or more botulinum toxin type is botulinum toxin type C2. In one aspect is provided such a composition for use, wherein the one or more botulinum toxin type is botulinum toxin type D. In one aspect is provided such a composition for use, wherein the one or more botulinum toxin type is botulinum toxin type E. In one aspect is provided such a composition for use, wherein the one or more botulinum toxin type E is EB-001A or EB-001T. In one aspect is provided such a composition for use, wherein the one or more botulinum toxin type E is EB-001A. In one aspect is provided such a composition for use, wherein the one or more botulinum toxin type E is EB-001T. In one aspect is provided such a composition for use, wherein the one or more botulinum toxin type is botulinum toxin type F. In one aspect is provided such a composition for use, wherein the one or more botulinum toxin type is botulinum toxin type G. In one aspect is provided such a composition for use, wherein the skin condition in the subject is selected from acne vulgaris, acne rosacea type 1, acne rosacea type 2, psoriasis, and hyperhidrosis. In one aspect is provided such a composition for use, wherein the skin condition in the subject is acne vulgaris. In one aspect is provided such a composition for use, wherein the skin condition in the subject is acne rosacea type 1. In one aspect is provided such a composition for use, wherein the skin condition in the subject is acne rosacea type 2. In one aspect is provided such a composition for use, wherein the skin condition in the subject is psoriasis. In one aspect is provided such a composition for use, wherein the skin condition in the subject is hyperhidrosis. In one aspect is provided such a composition for use, wherein the composition is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In one aspect is provided such a composition for use, wherein the composition is applied to the skin of the subject once per week for 2 weeks. In one aspect is provided such a composition for use, wherein the composition is applied to the skin of the subject once per week for 3 weeks. In one aspect is provided such a composition for use, wherein the composition is applied to the skin of the subject once per week for 4 weeks. In one aspect is provided such a composition for use, wherein the composition is applied to the skin of the subject once per week for 5 weeks. In one aspect is provided such a composition for use, wherein the composition comprising Spongilla is applied to the skin of the subject once per week for 6 weeks. In one aspect is provided such a composition for use, wherein the composition is applied to the skin of the subject once per week for 7 weeks. In one aspect is provided such a composition for use, wherein the composition is applied to the skin of the subject once per week for 8 weeks.

A composition comprising Spongilla and one or more botulinum toxins for use in the treatment of hyperhidrosis in a subject in need thereof. In one aspect is provided such a composition for use, wherein botulinum toxin is selected from botulinum toxin type A, botulinum toxin type B, botulinum toxin type C1, botulinum toxin type C2, botulinum toxin type D, botulinum toxin type E, botulinum toxin type F and botulinum toxin type G. In one aspect is provided such a composition for use wherein the one or more botulinum toxin type is selected from botulinum toxin type A, botulinum toxin type B, and botulinum toxin type E. In one aspect is provided such a composition for use wherein the one or more botulinum toxin type is botulinum toxin type A. In one aspect is provided such a composition for use, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a composition for use wherein the botulinum toxin type A is onabotulinumtoxinA. In one aspect is provided such a composition for use wherein the botulinum toxin type A is abobotulinumtoxinA. In one aspect is provided such a composition for use, wherein the botulinum toxin type A is incobotulinumtoxinA. In one aspect is provided such a composition for use, wherein the botulinum toxin type A is prabotulinumtoxinA. In one aspect is provided such a composition for use, wherein the one or more botulinum toxin type is botulinum toxin type B. In another aspect is provided any of the methods disclosed herein, wherein the one or more botulinum toxin type B is rimabotulinumtoxinB. In one aspect is provided such a composition for use, wherein the one or more botulinum toxin type is botulinum toxin type C1. In one aspect is provided such a composition for use, wherein the one or more botulinum toxin type is botulinum toxin type C2. In one aspect is provided such a composition for use, wherein the one or more botulinum toxin type is botulinum toxin type D. In one aspect is provided such a composition for use, wherein the one or more botulinum toxin type is botulinum toxin type E. In one aspect is provided such a composition for use, wherein the one or more botulinum toxin type E is EB-001A or EB-001T. In one aspect is provided such a composition for use, wherein the one or more botulinum toxin type E is EB-001A. In one aspect is provided such a composition for use, wherein the one or more botulinum toxin type E is EB-001T. In one aspect is provided such a composition for use, wherein the one or more botulinum toxin type is botulinum toxin type F. In one aspect is provided such a composition for use, wherein the one or more botulinum toxin type is botulinum toxin type G.

A composition comprising Spongilla and one or more botulinum toxins for use in the treatment of hyperhidrosis in a subject in need thereof. In one aspect is provided such a composition for use, wherein botulinum toxin is selected from botulinum toxin type A, botulinum toxin type B, botulinum toxin type C1, botulinum toxin type C2, botulinum toxin type D, botulinum toxin type E, botulinum toxin type F and botulinum toxin type G. In one aspect is provided such a composition for use wherein the one or more botulinum toxin type is selected from botulinum toxin type A, botulinum toxin type B, and botulinum toxin type E.

A composition comprising Spongilla and one or more botulinum toxins type A for use in the treatment of hyperhidrosis in a subject in need thereof, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a composition for use wherein the botulinum toxin type A is onabotulinumtoxinA. In one aspect is provided such a composition for use wherein the botulinum toxin type A is abobotulinumtoxinA. In one aspect is provided such a composition for use, wherein the botulinum toxin type A is incobotulinumtoxinA. In one aspect is provided such a composition for use, wherein the botulinum toxin type A is prabotulinumtoxinA.

A composition comprising Spongilla and onabotulinumtoxinA for use in the treatment of hyperhidrosis in a subject in need thereof. In another aspect is provided such a composition wherein the composition is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In another aspect is provided such a composition wherein the composition is applied to the skin of the subject once per week for 2 weeks. In another aspect is provided such a composition wherein the composition is applied to the skin of the subject once per week for 3 weeks. In another aspect is provided such a composition wherein the composition is applied to the skin of the subject once per week for 4 weeks. In another aspect is provided such a composition wherein the composition is applied to the skin of the subject once per week for 5 weeks. In another aspect is provided such a composition wherein the composition is applied to the skin of the subject once per week for 6 weeks. In another aspect is provided such a composition wherein the composition is applied to the skin of the subject once per week for 7 weeks. In another aspect is provided such a composition wherein the composition is applied to the skin of the subject is applied to the skin of the subject once per week for 8 weeks.

A first composition comprising Spongilla for use in reducing sweat production in a subject wherein the method comprises administering to the subject an effective amount of the first composition in combination with an effective amount of a second composition, the second composition comprising one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided such a first composition wherein the subject experiences a greater than 10% reduction in gravimetrically-measured sweat production following administration of the first composition and the second composition compared to the subject's gravimetrically-measured sweat production prior to such administration. In another aspect is provided such a composition for use, wherein the subject experiences a greater than 20%, 30%, 40%, 50%, 60%, 70%, 80%, 90%, or 95% reduction in gravimetrically-measured sweat production following administration of the first composition and the second composition compared to the subject's gravimetrically-measured sweat production prior to treatment. In another aspect is provided such a composition for use, wherein the subject experiences a greater than 30% reduction in gravimetrically-measured sweat production following administration of the first composition and the second composition compared to the subject's gravimetrically-measured sweat production prior to treatment. In another aspect is provided such a composition for use, wherein the subject experiences a greater than 40% reduction in gravimetrically-measured sweat production following administration of the first composition and the second composition compared to the subject's gravimetrically-measured sweat production prior to treatment. In another aspect is provided such a composition for use, wherein the subject experiences a greater than 50% reduction in gravimetrically-measured sweat production following administration of the first composition and the second composition compared to the subject's gravimetrically-measured sweat production prior to treatment. In another aspect is provided such a composition for use, wherein the subject experiences a greater than 60% reduction in gravimetrically-measured sweat production following administration of the first composition and the second composition compared to the subject's gravimetrically-measured sweat production prior to treatment. In another aspect is provided such a composition for use, wherein the subject experiences a greater than 70% reduction in gravimetrically-measured sweat production following administration of the first composition and the second composition compared to the subject's gravimetrically-measured sweat production prior to treatment. In another aspect is provided such a composition for use, wherein the subject experiences a greater than 80% reduction in gravimetrically-measured sweat production following administration of the first composition and the second composition compared to the subject's gravimetrically-measured sweat production prior to treatment. In another aspect is provided such a composition for use, wherein the subject experiences a greater than 90% reduction in gravimetrically-measured sweat production following administration of the first composition and the second composition compared to the subject's gravimetrically-measured sweat production prior to treatment. In another aspect is provided such a composition for use, wherein the subject experiences a greater than 95% reduction in gravimetrically-measured sweat production following administration of the first composition and the second composition compared to the subject's gravimetrically-measured sweat production prior to treatment. In another aspect is provided such a composition for use, wherein the subject is treated once per month, twice per month, three times per month, four times per month, five times per month, six times per month, 7 times per month, 8 times per month, 9 times per month, 10 times per month, 11 times per month, 12 times per month, 13 times per month, 14 times per month, 15 times per month, 16 times per month, 17 times per month, 18 times per month, 19 times per month, 20 times per month, 21 times per month, 22 times per month, or 24 times per month. In another aspect is provided such a composition for use, wherein the subject is treated twice per month. In another aspect is provided such a composition for use, wherein the subject is treated three times per month. In another aspect is provided such a composition for use, wherein the subject is treated once per week. In another aspect is provided such a composition for use, wherein the subject is at least 18 years old. In another aspect is provided such a composition for use, wherein the subject is treated once per month, or once every two months, or once every three months, or once every four months, or once every five months, or once every six months, or once every seven months, or once every eight months, or once every nine months, or once every 10 months, or once every 11 months, or once every 12 months. In another aspect is provided such a composition for use, wherein the subject is treated once per month. In another aspect is provided such a composition for use, wherein the subject is treated once every two months. In another aspect is provided such a composition for use, wherein the subject is treated once every three months. In another aspect is provided such a composition for use, wherein the subject is treated once every four months. In another aspect is provided such a composition for use, wherein the subject is treated once every five months. In another aspect is provided such a composition for use, wherein the subject is treated once every six months. In another aspect is provided such a composition for use, wherein the subject is treated once every seven months. In another aspect is provided such a composition for use, wherein the subject is treated once every eight months. In another aspect is provided such a composition for use, wherein the subject is treated once every nine months. In another aspect is provided such a composition for use, wherein the subject is treated once every 10 months. In another aspect is provided such a composition for use, wherein the subject is treated once every 11 months. In another aspect is provided such a composition for use, wherein the subject is treated once every 12 months. In another aspect is provided such a composition for use, wherein the subject suffers from primary axillary hyperhidrosis.

A first composition comprising Spongilla for use in reducing sweat production in a subject wherein the method comprises administering to the subject an effective amount of the first composition in combination with an effective amount of a second composition, the second composition comprising one or more botulinum toxin type selected from botulinum toxin type A. In one aspect is provided such a first composition for use, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a first composition for use wherein the botulinum toxin type A is onabotulinumtoxinA. In one aspect is provided such a first composition for use wherein the botulinum toxin type A is abobotulinumtoxinA. In one aspect is provided such a first composition for use, wherein the botulinum toxin type A is incobotulinumtoxinA. In one aspect is provided such a first composition for use, wherein the botulinum toxin type A is prabotulinumtoxinA. In another aspect is provided such a first composition wherein the subject experiences a greater than 10% reduction in gravimetrically-measured sweat production following administration of the first composition and the second composition compared to the subject's gravimetrically-measured sweat production prior to such administration. In another aspect is provided such a composition for use, wherein the subject experiences a greater than 20%, 30%, 40%, 50%, 60%, 70%, 80%, 90%, or 95% reduction in gravimetrically-measured sweat production following administration of the first composition and the second composition compared to the subject's gravimetrically-measured sweat production prior to treatment. In another aspect is provided such a composition for use, wherein the subject experiences a greater than 30% reduction in gravimetrically-measured sweat production following administration of the first composition and the second composition compared to the subject's gravimetrically-measured sweat production prior to treatment. In another aspect is provided such a composition for use, wherein the subject experiences a greater than 40% reduction in gravimetrically-measured sweat production following administration of the first composition and the second composition compared to the subject's gravimetrically-measured sweat production prior to treatment. In another aspect is provided such a composition for use, wherein the subject experiences a greater than 50% reduction in gravimetrically-measured sweat production following administration of the first composition and the second composition compared to the subject's gravimetrically-measured sweat production prior to treatment. In another aspect is provided such a composition for use, wherein the subject experiences a greater than 60% reduction in gravimetrically-measured sweat production following administration of the first composition and the second composition compared to the subject's gravimetrically-measured sweat production prior to treatment. In another aspect is provided such a composition for use, wherein the subject experiences a greater than 70% reduction in gravimetrically-measured sweat production following administration of the first composition and the second composition compared to the subject's gravimetrically-measured sweat production prior to treatment. In another aspect is provided such a composition for use, wherein the subject experiences a greater than 80% reduction in gravimetrically-measured sweat production following administration of the first composition and the second composition compared to the subject's gravimetrically-measured sweat production prior to treatment. In another aspect is provided such a composition for use, wherein the subject experiences a greater than 90% reduction in gravimetrically-measured sweat production following administration of the first composition and the second composition compared to the subject's gravimetrically-measured sweat production prior to treatment. In another aspect is provided such a composition for use, wherein the subject experiences a greater than 95% reduction in gravimetrically-measured sweat production following administration of the first composition and the second composition compared to the subject's gravimetrically-measured sweat production prior to treatment. In another aspect is provided such a composition for use, wherein the subject is treated once per month, twice per month, three times per month, four times per month, five times per month, six times per month, 7 times per month, 8 times per month, 9 times per month, 10 times per month, 11 times per month, 12 times per month, 13 times per month, 14 times per month, 15 times per month, 16 times per month, 17 times per month, 18 times per month, 19 times per month, 20 times per month, 21 times per month, 22 times per month, or 24 times per month. In another aspect is provided such a composition for use, wherein the subject is treated twice per month. In another aspect is provided such a composition for use, wherein the subject is treated three times per month. In another aspect is provided such a composition for use, wherein the subject is treated once per week. In another aspect is provided such a composition for use, wherein the subject is at least 18 years old. In another aspect is provided such a composition for use, wherein the subject is treated once per month, or once every two months, or once every three months, or once every four months, or once every five months, or once every six months, or once every seven months, or once every eight months, or once every nine months, or once every 10 months, or once every 11 months, or once every 12 months. In another aspect is provided such a composition for use, wherein the subject is treated once per month. In another aspect is provided such a composition for use, wherein the subject is treated once every two months. In another aspect is provided such a composition for use, wherein the subject is treated once every three months. In another aspect is provided such a composition for use, wherein the subject is treated once every four months. In another aspect is provided such a composition for use, wherein the subject is treated once every five months. In another aspect is provided such a composition for use, wherein the subject is treated once every six months. In another aspect is provided such a composition for use, wherein the subject is treated once every seven months. In another aspect is provided such a composition for use, wherein the subject is treated once every eight months. In another aspect is provided such a composition for use, wherein the subject is treated once every nine months. In another aspect is provided such a composition for use, wherein the subject is treated once every 10 months. In another aspect is provided such a composition for use, wherein the subject is treated once every 11 months. In another aspect is provided such a composition for use, wherein the subject is treated once every 12 months. In another aspect is provided such a composition for use, wherein the subject suffers from primary axillary hyperhidrosis.

A first composition comprising Spongilla for use in reducing sweat production in a subject wherein the method comprises administering to the subject an effective amount of the first composition in combination with an effective amount of a second composition, the second composition comprising onabotulinumtoxinA. In another aspect is provided such a first composition wherein the subject experiences a greater than 10% reduction in gravimetrically-measured sweat production following administration of the first composition and the second composition compared to the subject's gravimetrically-measured sweat production prior to such administration. In another aspect is provided such a composition for use, wherein the subject experiences a greater than 20%, 30%, 40%, 50%, 60%, 70%, 80%, 90%, or 95% reduction in gravimetrically-measured sweat production following administration of the first composition and the second composition compared to the subject's gravimetrically-measured sweat production prior to treatment. In another aspect is provided such a composition for use, wherein the subject experiences a greater than 30% reduction in gravimetrically-measured sweat production following administration of the first composition and the second composition compared to the subject's gravimetrically-measured sweat production prior to treatment. In another aspect is provided such a composition for use, wherein the subject experiences a greater than 40% reduction in gravimetrically-measured sweat production following administration of the first composition and the second composition compared to the subject's gravimetrically-measured sweat production prior to treatment. In another aspect is provided such a composition for use, wherein the subject experiences a greater than 50% reduction in gravimetrically-measured sweat production following administration of the first composition and the second composition compared to the subject's gravimetrically-measured sweat production prior to treatment. In another aspect is provided such a composition for use, wherein the subject experiences a greater than 60% reduction in gravimetrically-measured sweat production following administration of the first composition and the second composition compared to the subject's gravimetrically-measured sweat production prior to treatment. In another aspect is provided such a composition for use, wherein the subject experiences a greater than 70% reduction in gravimetrically-measured sweat production following administration of the first composition and the second composition compared to the subject's gravimetrically-measured sweat production prior to treatment. In another aspect is provided such a composition for use, wherein the subject experiences a greater than 80% reduction in gravimetrically-measured sweat production following administration of the first composition and the second composition compared to the subject's gravimetrically-measured sweat production prior to treatment. In another aspect is provided such a composition for use, wherein the subject experiences a greater than 90% reduction in gravimetrically-measured sweat production following administration of the first composition and the second composition compared to the subject's gravimetrically-measured sweat production prior to treatment. In another aspect is provided such a composition for use, wherein the subject experiences a greater than 95% reduction in gravimetrically-measured sweat production following administration of the first composition and the second composition compared to the subject's gravimetrically-measured sweat production prior to treatment. In another aspect is provided such a composition for use, wherein the subject is treated once per month, twice per month, three times per month, four times per month, five times per month, six times per month, 7 times per month, 8 times per month, 9 times per month, 10 times per month, 11 times per month, 12 times per month, 13 times per month, 14 times per month, 15 times per month, 16 times per month, 17 times per month, 18 times per month, 19 times per month, 20 times per month, 21 times per month, 22 times per month, or 24 times per month. In another aspect is provided such a composition for use, wherein the subject is treated twice per month. In another aspect is provided such a composition for use, wherein the subject is treated three times per month. In another aspect is provided such a composition for use, wherein the subject is treated once per week. In another aspect is provided such a composition for use, wherein the subject is at least 18 years old. In another aspect is provided such a composition for use, wherein the subject is treated once per month, or once every two months, or once every three months, or once every four months, or once every five months, or once every six months, or once every seven months, or once every eight months, or once every nine months, or once every 10 months, or once every 11 months, or once every 12 months. In another aspect is provided such a composition for use, wherein the subject is treated once per month. In another aspect is provided such a composition for use, wherein the subject is treated once every two months. In another aspect is provided such a composition for use, wherein the subject is treated once every three months. In another aspect is provided such a composition for use, wherein the subject is treated once every four months. In another aspect is provided such a composition for use, wherein the subject is treated once every five months. In another aspect is provided such a composition for use, wherein the subject is treated once every six months. In another aspect is provided such a composition for use, wherein the subject is treated once every seven months. In another aspect is provided such a composition for use, wherein the subject is treated once every eight months. In another aspect is provided such a composition for use, wherein the subject is treated once every nine months. In another aspect is provided such a composition for use, wherein the subject is treated once every 10 months. In another aspect is provided such a composition for use, wherein the subject is treated once every 11 months. In another aspect is provided such a composition for use, wherein the subject is treated once every 12 months. In another aspect is provided such a composition for use, wherein the subject suffers from primary axillary hyperhidrosis.

A first composition comprising Spongilla for use in a method of treating hyperhidrosis in a subject in need thereof, wherein the method comprises administering to the subject an effective amount of the first composition in combination with an effective amount of a second composition, the second composition comprising botulinum toxin type A. In one aspect is provided such a first composition for use, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a first composition for use wherein the botulinum toxin type A is onabotulinumtoxinA. In one aspect is provided such a first composition for use wherein the botulinum toxin type A is abobotulinumtoxinA. In one aspect is provided such a first composition for use, wherein the botulinum toxin type A is incobotulinumtoxinA. In one aspect is provided such a first composition for use, wherein the botulinum toxin type A is prabotulinumtoxinA. In one aspect is provided such a first composition for use, wherein the first composition comprising Spongilla is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In one aspect is provided such a first composition for use, wherein the first composition comprising Spongilla is applied to the skin of the subject once per week for 2 weeks. In one aspect is provided such a first composition for use, wherein the first composition comprising Spongilla is applied to the skin of the subject once per week for 3 weeks. In one aspect is provided such a first composition for use, wherein the first composition comprising Spongilla is applied to the skin of the subject once per week for 4 weeks. In one aspect is provided such a first composition for use, wherein the first composition comprising Spongilla is applied to the skin of the subject once per week for 5 weeks. In one aspect is provided such a first composition for use, wherein the first composition comprising Spongilla is applied to the skin of the subject once per week for 6 weeks. In one aspect is provided such a first composition for use, wherein the first composition comprising Spongilla is applied to the skin of the subject once per week for 7 weeks. In one aspect is provided such a first composition for use, wherein the first composition comprising Spongilla is applied to the skin of the subject once per week for 8 weeks.

A first composition comprising Spongilla for use in a method of treating hyperhidrosis in a subject in need thereof, wherein the method comprises administering to the subject an effective amount of the first composition in combination with an effective amount of a second composition, the second composition comprising onabotulinumtoxinA. In one aspect is provided such a first composition for use, wherein the first composition comprising Spongilla is applied to the skin of the subject at least at once per week for at least two weeks, at least three weeks, at least once per week for at least 4 weeks, at least once per week for at least 5 weeks, at least once per week for at least 6 weeks, at least once per week for at least 7 weeks, at least at once per week for at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In one aspect is provided such a first composition for use, wherein the first composition comprising Spongilla is applied to the skin of the subject once per week for 2 weeks. In one aspect is provided such a first composition for use, wherein the first composition comprising Spongilla is applied to the skin of the subject once per week for 3 weeks. In one aspect is provided such a first composition for use, wherein the first composition comprising Spongilla is applied to the skin of the subject once per week for 4 weeks. In one aspect is provided such a first composition for use, wherein the first composition comprising Spongilla is applied to the skin of the subject once per week for 5 weeks. In one aspect is provided such a first composition for use, wherein the first composition comprising Spongilla is applied to the skin of the subject once per week for 6 weeks. In one aspect is provided such a first composition for use, wherein the first composition comprising Spongilla is applied to the skin of the subject once per week for 7 weeks. In one aspect is provided such a first composition for use, wherein the first composition comprising Spongilla is applied to the skin of the subject once per week for 8 weeks.

A composition comprising Spongilla and one or more botulinum toxins for the treatment of a skin condition or disease in a subject in need thereof, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another aspect is provided such a first composition for use, wherein the skin condition or condition in the subject is hyperhidrosis, acne vulgaris and rosacea, and wherein the botulinum toxin is selected from botulinum toxin type A, botulinum toxin type B, botulinum toxin type C1, botulinum toxin type C2, botulinum toxin type D, botulinum toxin type E, botulinum toxin type F and botulinum toxin type G, and wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another aspect is provided such a composition for use, wherein the skin condition or condition in the subject is hyperhidrosis, acne vulgaris and rosacea. In one aspect is provided such a composition for use, wherein botulinum toxin is selected from botulinum toxin type A, botulinum toxin type B, botulinum toxin type C1, botulinum toxin type C2, botulinum toxin type D, botulinum toxin type E, botulinum toxin type F and botulinum toxin type G. In one aspect is provided such a composition for use wherein the one or more botulinum toxin type is selected from botulinum toxin type A, botulinum toxin type B, and botulinum toxin type E. In one aspect is provided such a composition for use wherein the one or more botulinum toxin type is botulinum toxin type A. In one aspect is provided such a composition for use, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a composition for use wherein the botulinum toxin type A is onabotulinumtoxinA. In one aspect is provided such a composition for use wherein the botulinum toxin type A is abobotulinumtoxinA. In one aspect is provided such a composition for use, wherein the botulinum toxin type A is incobotulinumtoxinA. In one aspect is provided such a composition for use, wherein the botulinum toxin type A is prabotulinumtoxinA. In one aspect is provided such a composition for use, wherein the one or more botulinum toxin type is botulinum toxin type B. In another aspect is provided any of the methods disclosed herein, wherein the one or more botulinum toxin type B is rimabotulinumtoxinB. In one aspect is provided such a composition for use, wherein the one or more botulinum toxin type is botulinum toxin type C1. In one aspect is provided such a composition for use, wherein the one or more botulinum toxin type is botulinum toxin type C2. In one aspect is provided such a composition for use, wherein the one or more botulinum toxin type is botulinum toxin type D. In one aspect is provided such a composition for use, wherein the one or more botulinum toxin type is botulinum toxin type E. In one aspect is provided such a composition for use, wherein the one or more botulinum toxin type E is EB-001A or EB-001T. In one aspect is provided such a composition for use, wherein the one or more botulinum toxin type E is EB-001A. In one aspect is provided such a composition for use, wherein the one or more botulinum toxin type E is EB-001T. In one aspect is provided such a composition for use, wherein the one or more botulinum toxin type is botulinum toxin type F. In one aspect is provided such a composition for use, wherein the one or more botulinum toxin type is botulinum toxin type G. In one aspect is provided such a composition for use, wherein the skin condition in the subject is selected from acne vulgaris, acne rosacea type 1, acne rosacea type 2, psoriasis, and hyperhidrosis. In one aspect is provided such a composition for use, wherein the skin condition in the subject is acne vulgaris. In one aspect is provided such a composition for use, wherein the skin condition in the subject is acne rosacea type 1. In one aspect is provided such a composition for use, wherein the skin condition in the subject is acne rosacea type 2. In one aspect is provided such a composition for use, wherein the skin condition in the subject is psoriasis. In one aspect is provided such a composition for use, wherein the skin condition in the subject is hyperhidrosis. In one aspect is provided such a composition for use, wherein the composition is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In one aspect is provided such a composition for use, wherein the composition is applied to the skin of the subject once per week for 2 weeks. In one aspect is provided such a composition for use, wherein the composition is applied to the skin of the subject once per week for 3 weeks. In one aspect is provided such a composition for use, wherein the composition is applied to the skin of the subject once per week for 4 weeks. In one aspect is provided such a composition for use, wherein the composition is applied to the skin of the subject once per week for 5 weeks. In one aspect is provided such a composition for use, wherein the composition comprising Spongilla is applied to the skin of the subject once per week for 6 weeks. In one aspect is provided such a composition for use, wherein the composition is applied to the skin of the subject once per week for 7 weeks. In one aspect is provided such a composition for use, wherein the composition is applied to the skin of the subject once per week for 8 weeks.

A composition comprising Spongilla and one or more botulinum toxins for use in the treatment of hyperhidrosis in a subject in need thereof. In one aspect is provided such a composition for use, wherein botulinum toxin is selected from botulinum toxin type A, botulinum toxin type B, botulinum toxin type C1, botulinum toxin type C2, botulinum toxin type D, botulinum toxin type E, botulinum toxin type F and botulinum toxin type G. In one aspect is provided such a composition for use wherein the one or more botulinum toxin type is selected from botulinum toxin type A, botulinum toxin type B, and botulinum toxin type E. In one aspect is provided such a composition for use wherein the one or more botulinum toxin type is botulinum toxin type A. In one aspect is provided such a composition for use, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a composition for use wherein the botulinum toxin type A is onabotulinumtoxinA. In one aspect is provided such a composition for use wherein the botulinum toxin type A is abobotulinumtoxinA. In one aspect is provided such a composition for use, wherein the botulinum toxin type A is incobotulinumtoxinA. In one aspect is provided such a composition for use, wherein the botulinum toxin type A is prabotulinumtoxinA. In one aspect is provided such a composition for use, wherein the one or more botulinum toxin type is botulinum toxin type B. In another aspect is provided any of the methods disclosed herein, wherein the one or more botulinum toxin type B is rimabotulinumtoxinB. In one aspect is provided such a composition for use, wherein the one or more botulinum toxin type is botulinum toxin type C1. In one aspect is provided such a composition for use, wherein the one or more botulinum toxin type is botulinum toxin type C2. In one aspect is provided such a composition for use, wherein the one or more botulinum toxin type is botulinum toxin type D. In one aspect is provided such a composition for use, wherein the one or more botulinum toxin type is botulinum toxin type E. In one aspect is provided such a composition for use, wherein the one or more botulinum toxin type E is EB-001A or EB-001T. In one aspect is provided such a composition for use, wherein the one or more botulinum toxin type E is EB-001A. In one aspect is provided such a composition for use, wherein the one or more botulinum toxin type E is EB-001T. In one aspect is provided such a composition for use, wherein the one or more botulinum toxin type is botulinum toxin type F. In one aspect is provided such a composition for use, wherein the one or more botulinum toxin type is botulinum toxin type G.

A composition comprising Spongilla and one or more botulinum toxins for use in the treatment of hyperhidrosis in a subject in need thereof. In one aspect is provided such a composition for use, wherein botulinum toxin is selected from botulinum toxin type A, botulinum toxin type B, botulinum toxin type C1, botulinum toxin type C2, botulinum toxin type D, botulinum toxin type E, botulinum toxin type F and botulinum toxin type G. In one aspect is provided such a composition for use wherein the one or more botulinum toxin type is selected from botulinum toxin type A, botulinum toxin type B, and botulinum toxin type E.

A composition comprising Spongilla and one or more botulinum toxins type A for use in the treatment of hyperhidrosis in a subject in need thereof, wherein the botulinum toxin type A is selected from onabotulinumtoxinA, abobotulinumtoxinA, incobotulinumtoxinA, prabotulinumtoxinA, and daxibotulinumtoxinA. In one aspect is provided such a composition for use wherein the botulinum toxin type A is onabotulinumtoxinA. In one aspect is provided such a composition for use wherein the botulinum toxin type A is abobotulinumtoxinA. In one aspect is provided such a composition for use, wherein the botulinum toxin type A is incobotulinumtoxinA. In one aspect is provided such a composition for use, wherein the botulinum toxin type A is prabotulinumtoxinA.

A composition comprising Spongilla and onabotulinumtoxinA for use in the treatment of hyperhidrosis in a subject in need thereof. In another aspect is provided such a composition wherein the composition is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In another aspect is provided such a composition wherein the composition is applied to the skin of the subject once per week for 2 weeks. In another aspect is provided such a composition wherein the composition is applied to the skin of the subject once per week for 3 weeks. In another aspect is provided such a composition wherein the composition is applied to the skin of the subject once per week for 4 weeks. In another aspect is provided such a composition wherein the composition is applied to the skin of the subject once per week for 5 weeks. In another aspect is provided such a composition wherein the composition is applied to the skin of the subject once per week for 6 weeks. In another aspect is provided such a composition wherein the composition is applied to the skin of the subject once per week for 7 weeks. In another aspect is provided such a composition wherein the composition is applied to the skin of the subject is applied to the skin of the subject once per week for 8 weeks.

In one aspect are provided methods of hyperhidrosis in a subject, comprising applying to the skin of the subject in need thereof an effective amount of a first composition comprising Spongilla, and an effective amount of a second composition comprising onabotulinumtoxinA. In another aspect is provided any of the methods, wherein the first composition comprising Spongilla is applied to the skin of the subject at least at once per week for at least two weeks, at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In another aspect is provided any of the methods disclosed herein, wherein the first composition comprising Spongilla is applied to the skin of the subject once per week for 2 weeks. In another aspect is provided any of the methods disclosed herein, wherein the first composition comprising Spongilla is applied to the skin of the subject once per week for 3 weeks. In another aspect is provided any of the methods disclosed herein, wherein the first composition comprising Spongilla is applied to the skin of the subject once per week for 4 weeks. In another aspect is provided any of the methods disclosed herein, wherein the first composition comprising Spongilla is applied to the skin of the subject once per week for 5 weeks. In another aspect is provided any of the methods disclosed herein, wherein the first composition comprising Spongilla is applied to the skin of the subject once per week for 6 weeks. In another aspect is provided any of the methods disclosed herein, wherein the first composition comprising Spongilla is applied to the skin of the subject once per week for 7 weeks. In another aspect is provided any of the methods disclosed herein, wherein the first composition comprising Spongilla is applied to the skin of the subject once per week for 8 weeks.

In another aspect provided herein are any of the disclosed methods, wherein the method comprises comprising applying to the skin of the subject in need thereof an effective amount of a first composition comprising Spongilla, and an effective amount of a second composition comprising one or more botulinum toxins.

In one aspect is provided a method of treating a skin condition in a subject, comprising applying to the skin of the subject a first composition comprising Spongilla, and a second composition comprising one or more botulinum toxins. In another aspect is provided any of the methods disclosed herein, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided any of the methods disclosed herein, wherein the one or more botulinum toxin type is selected from botulinum toxin type A, botulinum toxin type B, and botulinum toxin type E. In another aspect is provided any of the methods disclosed herein, wherein the one or more botulinum toxin type is botulinum toxin type A. In another aspect is provided any of the methods disclosed herein, wherein the botulinum toxin type A is selected from onabotulinumtoxinA, abobotulinumtoxinA, incobotulinumtoxinA, prabotulinumtoxinA, and daxibotulinumtoxinA. In another aspect is provided any of the methods disclosed herein, wherein the botulinum toxin type A is onabotulinumtoxinA. In another aspect is provided any of the methods disclosed herein, wherein the botulinum toxin type A is abobotulinumtoxinA. In another aspect is provided any of the methods disclosed herein, wherein the botulinum toxin type A is incobotulinumtoxinA. In another aspect is provided any of the methods disclosed herein, wherein the botulinum toxin type A is prabotulinumtoxinA. In another aspect is provided any of the methods disclosed herein, wherein the one or more botulinum toxin type is botulinum toxin type B. In another aspect is provided any of the methods disclosed herein, wherein the one or more botulinum toxin type B is rimabotulinumtoxinB. In another aspect is provided any of the methods disclosed herein, wherein the one or more botulinum toxin type is botulinum toxin type C1. In another aspect is provided any of the methods disclosed herein, wherein the one or more botulinum toxin type is botulinum toxin type C2. In another aspect is provided any of the methods disclosed herein, wherein the one or more botulinum toxin type is botulinum toxin type D. In another aspect is provided any of the methods disclosed herein, wherein the one or more botulinum toxin type is botulinum toxin type E. In another aspect is provided any of the methods disclosed herein, wherein the one or more botulinum toxin type E is EB-001A or EB-001T. In another aspect is provided any of the methods disclosed herein, wherein the one or more botulinum toxin type E is EB-001A. In another aspect is provided any of the methods disclosed herein, wherein the one or more botulinum toxin type E is EB-001T. In another aspect is provided any of the methods disclosed herein, wherein the one or more botulinum toxin type is botulinum toxin type F. In another aspect is provided any of the methods disclosed herein, wherein the one or more botulinum toxin type is botulinum toxin type G. In another aspect is provided any of the methods disclosed herein, wherein the skin condition in the subject is selected from acne vulgaris, acne rosacea type 1, acne rosacea type 2, psoriasis, and hyperhidrosis. In another aspect is provided any of the methods disclosed herein, wherein the skin condition in the subject is acne vulgaris. In another aspect is provided any of the methods disclosed herein, wherein the skin condition in the subject is acne rosacea type 1. In another aspect is provided any of the methods disclosed herein, wherein the skin condition in the subject is acne rosacea type 2. In another aspect is provided any of the methods disclosed herein, wherein the skin condition in the subject is psoriasis. In another aspect is provided any of the methods disclosed herein, wherein the skin condition in the subject is hyperhidrosis.

In another aspect is provided a composition comprising a first composition and a second composition for use as a medicament, wherein the (a) the first composition comprises a Spongilla; and (b) the second composition one or more botulinum toxins. In another aspect is provided a composition comprising a first composition and a second composition for use as a medicament, wherein the (a) the first composition comprises a Spongilla lacustris; and (b) the second composition one or more botulinum toxins. In another aspect is provided such compositions for use as a medicament for the treatment of a skin condition in a subject. In another aspect is such combinations for use as a medicament for the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, and eccrine nevus. In another aspect is provided any of the compositions disclosed herein, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided any of the compositions disclosed herein, wherein the one or more botulinum toxin type is selected from botulinum toxin type A, botulinum toxin type B, and botulinum toxin type E. In another aspect is provided any of the compositions disclosed herein, wherein the one or more botulinum toxin type is botulinum toxin type A. In another aspect is provided any of the compositions disclosed herein, wherein the botulinum toxin type A is selected from onabotulinumtoxinA, abobotulinumtoxinA, incobotulinumtoxinA, prabotulinumtoxinA, and daxibotulinumtoxinA. In another aspect is provided any of the compositions disclosed herein, wherein the botulinum toxin type A is onabotulinumtoxinA. In another aspect is provided any of the compositions disclosed herein, wherein the botulinum toxin type A is abobotulinumtoxinA. In another aspect is provided any of the compositions disclosed herein, wherein the botulinum toxin type A is incobotulinumtoxinA. In another aspect is provided any of the compositions disclosed herein, wherein the botulinum toxin type A is prabotulinumtoxinA. In another aspect is provided any of the compositions disclosed herein, wherein the one or more botulinum toxin type is botulinum toxin type B. In another aspect is provided any of the methods disclosed herein, wherein the one or more botulinum toxin type B is rimabotulinumtoxinB. In another aspect is provided any of the compositions disclosed herein, wherein the one or more botulinum toxin type is botulinum toxin type C1. In another aspect is provided any of the compositions disclosed herein, wherein the one or more botulinum toxin type is botulinum toxin type C2. In another aspect is provided any of the compositions disclosed herein, wherein the one or more botulinum toxin type is botulinum toxin type D. In another aspect is provided any of the compositions disclosed herein, wherein the one or more botulinum toxin type is botulinum toxin type E. In another aspect is provided any of the compositions disclosed herein, wherein the one or more botulinum toxin type E is EB-001A or EB-001T. In another aspect is provided any of the compositions disclosed herein, wherein the one or more botulinum toxin type E is EB-001A. In another aspect is provided any of the compositions disclosed herein, wherein the one or more botulinum toxin type E is EB-001T. In another aspect is provided any of the compositions disclosed herein, wherein the one or more botulinum toxin type is botulinum toxin type F. In another aspect is provided any of the compositions disclosed herein, wherein the one or more botulinum toxin type is botulinum toxin type G. In another aspect is provided any of the compositions disclosed herein, wherein the skin condition in the subject is selected from acne vulgaris, acne rosacea type 1, acne rosacea type 2, psoriasis, and hyperhidrosis. In another aspect is provided any of the compositions disclosed herein, wherein the skin condition in the subject is acne vulgaris. In another aspect is provided any of the compositions disclosed herein, wherein the skin condition in the subject is acne rosacea type 1. In another aspect is provided any of the compositions disclosed herein, wherein the skin condition in the subject is acne rosacea type 2. In another aspect is provided any of the compositions disclosed herein, wherein the skin condition in the subject is psoriasis. In another aspect is provided any of the compositions disclosed herein, wherein the skin condition in the subject is hyperhidrosis. In another aspect is provided any of the compositions disclosed herein, wherein the second composition is selected from onabotulinumtoxinA, abobotulinumtoxinA, incobotulinumtoxinA, prabotulinumtoxinA, EB-001A, and EB-001T and the skin condition in the subject is acne rosacea type 1. In another aspect is provided any of the compositions disclosed herein, wherein the second composition is selected from onabotulinumtoxinA, abobotulinumtoxinA, incobotulinumtoxinA, prabotulinumtoxinA, EB-001A, and EB-001, and the skin condition in the subject is acne rosacea type 2. In another aspect is provided any of the compositions disclosed herein, wherein the second composition is selected from onabotulinumtoxinA, abobotulinumtoxinA, incobotulinumtoxinA, prabotulinumtoxinA, EB-001A, and EB-001 and the skin condition in the subject is psoriasis. In another aspect is provided any of the compositions disclosed herein, wherein the second composition is selected from onabotulinumtoxinA, abobotulinumtoxinA, incobotulinumtoxinA, prabotulinumtoxinA, EB-001A, and EB-001T and the skin condition in the subject is hyperhidrosis. In another aspect is provided any of the compositions disclosed herein, wherein the second composition is onabotulinumtoxinA, and the skin condition in the subject is acne rosacea type 1. In another aspect is provided any of the compositions disclosed herein, wherein the second composition is onabotulinumtoxinA, and the skin condition in the subject is acne rosacea type 2. In another aspect is provided any of the compositions disclosed herein, wherein the second composition is onabotulinumtoxinA, and the skin condition in the subject is psoriasis. In another aspect is provided any of the compositions disclosed herein, wherein the second composition is onabotulinumtoxinA, and the skin condition in the subject is hyperhidrosis.

In another aspect is provided a composition comprising a first composition and a second composition for use in the treatment of a skin condition in a subject, wherein (a) the first composition comprising a Spongilla; and (b) the second composition one or more botulinum toxins. In another aspect is provided a combination comprising a first composition and a second composition for use in the treatment of a skin condition in a subject, wherein (a) the first composition comprising a Spongilla lacustris; and (b) the second composition one or more botulinum toxins. In another aspect is such compositions for use in the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect is such compositions for use in the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect is such compositions for use in the treatment of a skin condition in a subject, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is such compositions for use in the treatment of a skin condition in a subject, wherein the one or more botulinum toxin type is selected from botulinum toxin type A, botulinum toxin type B, and botulinum toxin type E. In another aspect is such compositions for use in the treatment of a skin condition in a subject, wherein the one or more botulinum toxin type is botulinum toxin type A. In another aspect is such compositions for use in the treatment of a skin condition in a subject, wherein the botulinum toxin type A is selected from onabotulinumtoxinA, abobotulinumtoxinA, incobotulinumtoxinA, prabotulinumtoxinA, and daxibotulinumtoxinA. In another aspect is such compositions for use in the treatment of a skin condition in a subject, wherein the botulinum toxin type A is onabotulinumtoxinA. In another aspect is such compositions for use in the treatment of a skin condition in a subject, wherein the botulinum toxin type A is abobotulinumtoxinA. In another aspect is such compositions for use in the treatment of a skin condition in a subject, wherein the botulinum toxin type A is incobotulinumtoxinA. In another aspect is such compositions for use in the treatment of a skin condition in a subject, wherein the botulinum toxin type A is prabotulinumtoxinA. In another aspect is such compositions for use in the treatment of a skin condition in a subject, wherein the botulinum toxin type A is daxibotulinumtoxinA. In another aspect is such compositions for use in the treatment of a skin condition in a subject, wherein the one or more botulinum toxin type is botulinum toxin type B. In another aspect is provided any of the methods disclosed herein, wherein the one or more botulinum toxin type B is rimabotulinumtoxinB. In another aspect is such compositions for use in the treatment of a skin condition in a subject, wherein the one or more botulinum toxin type is botulinum toxin type C1. In another aspect is such compositions for use in the treatment of a skin condition in a subject, wherein the one or more botulinum toxin type is botulinum toxin type C2. In another aspect is such compositions for use in the treatment of a skin condition in a subject, wherein the one or more botulinum toxin type is botulinum toxin type D. In another aspect is such compositions for use in the treatment of a skin condition in a subject, wherein the one or more botulinum toxin type is botulinum toxin type E. In another aspect is such compositions for use in the treatment of a skin condition in a subject, wherein the one or more botulinum toxin type E is EB-001A or EB-001T. In another aspect is such compositions for use in the treatment of a skin condition in a subject, wherein the one or more botulinum toxin type E is EB-001A. In another aspect is such compositions for use in the treatment of a skin condition in a subject, wherein the one or more botulinum toxin type E is EB-001T. In another aspect is such compositions for use in the treatment of a skin condition in a subject, wherein the one or more botulinum toxin type is botulinum toxin type F. In another aspect is such compositions for use in the treatment of a skin condition in a subject, wherein the one or more botulinum toxin type is botulinum toxin type G. In another aspect is such compositions for use in the treatment of a skin condition in a subject, wherein the skin condition in the subject is selected from acne vulgaris, acne rosacea type 1, acne rosacea type 2, psoriasis, and hyperhidrosis. In another aspect is such compositions for use in the treatment of a skin condition in a subject, wherein the skin condition in the subject is acne vulgaris. In another aspect is provided any of the compositions disclosed herein, wherein the skin condition in the subject is acne rosacea type 1. In another aspect is such compositions for use in the treatment of a skin condition in a subject, wherein the skin condition in the subject is acne rosacea type 2. In another aspect is such compositions for use in the treatment of a skin condition in a subject, wherein the skin condition in the subject is psoriasis. In another aspect is such compositions for use in the treatment of a skin condition in a subject, wherein the skin condition in the subject is hyperhidrosis. In another aspect is such compositions for use in the treatment of a skin condition in a subject, wherein the second composition is selected from onabotulinumtoxinA, abobotulinumtoxinA, incobotulinumtoxinA, prabotulinumtoxinA, EB-001A, and EB-001T, and the skin condition in the subject is acne rosacea type 1. In another aspect is such compositions for use in the treatment of a skin condition in a subject, wherein the second composition is selected from onabotulinumtoxinA, abobotulinumtoxinA, incobotulinumtoxinA, prabotulinumtoxinA, EB-001A, and EB-001T, and the skin condition in the subject is acne rosacea type 2. In another aspect is such compositions for use in the treatment of a skin condition in a subject, wherein the second composition is selected from onabotulinumtoxinA, abobotulinumtoxinA, incobotulinumtoxinA, prabotulinumtoxinA, EB-001A, and EB-001T, and the skin condition in the subject is psoriasis. In another aspect is such compositions for use in the treatment of a skin condition in a subject, wherein the second composition is selected from onabotulinumtoxinA, abobotulinumtoxinA, incobotulinumtoxinA, prabotulinumtoxinA, EB-001A, and EB-001T, and the skin condition in the subject is hyperhidrosis. In another aspect is such compositions for use in the treatment of a skin condition in a subject, wherein the second composition is onabotulinumtoxinA, and the skin condition in the subject is acne rosacea type 1. In another aspect is such compositions for use in the treatment of a skin condition in a subject, wherein the second composition is onabotulinumtoxinA, and the skin condition in the subject is acne rosacea type 2. In another aspect is such compositions for use in the treatment of a skin condition in a subject, wherein the second composition is onabotulinumtoxinA, and the skin condition in the subject is psoriasis. In another aspect is such compositions for use in the treatment of a skin condition in a subject, wherein the second composition is onabotulinumtoxinA, and the skin condition in the subject is hyperhidrosis.

In another aspect is provided a composition for the manufacture of a medicament for the treatment of a skin condition in a subject, wherein the composition comprises a first composition and a second composition wherein (a) the first composition comprising a Spongilla; and (b) the second composition one or more botulinum toxins. In another aspect is provided a composition for the manufacture of a medicament for the treatment of a skin condition in a subject, wherein the composition comprises a first composition and a second composition wherein (a) the first composition comprising a Spongilla lacustris; and (b) the second composition one or more botulinum toxins, a composition for the manufacture of a medicament for the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect is provided a composition for the manufacture of a medicament for the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect is provided a composition for the manufacture of a medicament for the treatment of a skin condition in a subject, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect is provided a composition for the manufacture of a medicament for the treatment of a skin condition in a subject, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided a composition for the manufacture of a medicament for the treatment of a skin condition in a subject, wherein the one or more botulinum toxin type is selected from botulinum toxin type A, botulinum toxin type B, and botulinum toxin type E. In another aspect is provided a composition for the manufacture of a medicament for the treatment of a skin condition in a subject, wherein the one or more botulinum toxin type is botulinum toxin type A. In another aspect is provided a composition for the manufacture of a medicament for the treatment of a skin condition in a subject, wherein the botulinum toxin type A is selected from onabotulinumtoxinA, abobotulinumtoxinA, incobotulinumtoxinA, prabotulinumtoxinA, and daxibotulinumtoxinA. In another aspect is provided a composition for the manufacture of a medicament for the treatment of a skin condition in a subject, wherein the botulinum toxin type A is onabotulinumtoxinA. In another aspect is provided a composition for the manufacture of a medicament for the treatment of a skin condition in a subject, wherein the botulinum toxin type A is abobotulinumtoxinA. In another aspect is provided a composition for the manufacture of a medicament for the treatment of a skin condition in a subject, wherein the botulinum toxin type A is incobotulinumtoxinA. In another aspect is provided a composition for the manufacture of a medicament for the treatment of a skin condition in a subject, wherein the botulinum toxin type A is prabotulinumtoxinA. In another aspect is provided a composition for the manufacture of a medicament for the treatment of a skin condition in a subject, wherein the botulinum toxin type A is daxibotulinumtoxinA. In another aspect is provided a composition for the manufacture of a medicament for the treatment of a skin condition in a subject, wherein the one or more botulinum toxin type is botulinum toxin type B. In another aspect is provided any of the methods disclosed herein, wherein the one or more botulinum toxin type B is rimabotulinumtoxinB. In another aspect is provided a composition for the manufacture of a medicament for the treatment of a skin condition in a subject, wherein the one or more botulinum toxin type is botulinum toxin type C1. In another aspect is provided a composition for the manufacture of a medicament for the treatment of a skin condition in a subject, wherein the one or more botulinum toxin type is botulinum toxin type C2. In another aspect is provided a composition for the manufacture of a medicament for the treatment of a skin condition in a subject, wherein the one or more botulinum toxin type is botulinum toxin type D. In another aspect is provided a composition for the manufacture of a medicament for the treatment of a skin condition in a subject, wherein the one or more botulinum toxin type is botulinum toxin type E. In another aspect is provided a composition for the manufacture of a medicament for the treatment of a skin condition in a subject, wherein the one or more botulinum toxin type E is EB-001A or EB-001T. In another aspect is provided a composition for the manufacture of a medicament for the treatment of a skin condition in a subject, wherein the one or more botulinum toxin type E is EB-001A. In another aspect is provided a composition for the manufacture of a medicament for the treatment of a skin condition in a subject, wherein the one or more botulinum toxin type E is EB-001T. In another aspect is provided a composition for the manufacture of a medicament for the treatment of a skin condition in a subject, wherein the one or more botulinum toxin type is botulinum toxin type F. In another aspect is provided a composition for the manufacture of a medicament for the treatment of a skin condition in a subject, wherein the one or more botulinum toxin type is botulinum toxin type G. In another aspect is provided a composition for the manufacture of a medicament for the treatment of a skin condition in a subject, wherein the skin condition in the subject is selected from acne vulgaris, acne rosacea type 1, acne rosacea type 2, psoriasis, and hyperhidrosis. In another aspect is provided a composition for the manufacture of a medicament for the treatment of a skin condition in a subject, wherein the skin condition in the subject is acne vulgaris. In another aspect is provided a composition for the manufacture of a medicament for the treatment of a skin condition in a subject, wherein the skin condition in the subject is acne rosacea type 1. In another aspect is provided a composition for the manufacture of a medicament for the treatment of a skin condition in a subject, wherein the skin condition in the subject is acne rosacea type 2. In another aspect is provided a composition for the manufacture of a medicament for the treatment of a skin condition in a subject, wherein the skin condition in the subject is psoriasis. In another aspect is provided a composition for the manufacture of a medicament for the treatment of a skin condition in a subject, wherein the skin condition in the subject is hyperhidrosis. In another aspect is provided a composition for the manufacture of a medicament for the treatment of a skin condition in a subject, wherein the second composition is selected from onabotulinumtoxinA, abobotulinumtoxinA, incobotulinumtoxinA, prabotulinumtoxinA, EB-001A, and EB-001T, and the skin condition in the subject is acne rosacea type 1. In another aspect is provided a composition for the manufacture of a medicament for the treatment of a skin condition in a subject, wherein the second composition is selected from onabotulinumtoxinA, abobotulinumtoxinA, incobotulinumtoxinA, prabotulinumtoxinA, EB-001A, and EB-001T, and the skin condition in the subject is acne rosacea type 2. In another aspect is provided a composition for the manufacture of a medicament for the treatment of a skin condition in a subject, wherein the second composition is selected from onabotulinumtoxinA, abobotulinumtoxinA, incobotulinumtoxinA, prabotulinumtoxinA, EB-001A, and EB-001T, and the skin condition in the subject is psoriasis. In another aspect is provided a composition for the manufacture of a medicament for the treatment of a skin condition in a subject, wherein the second composition is selected from onabotulinumtoxinA, abobotulinumtoxinA, incobotulinumtoxinA, prabotulinumtoxinA, EB-001A, and EB-001T, and the skin condition in the subject is hyperhidrosis. In another aspect is provided a composition for the manufacture of a medicament for the treatment of a skin condition in a subject, wherein the second composition is onabotulinumtoxinA, and the skin condition in the subject is acne rosacea type 1. In another aspect is provided a composition for the manufacture of a medicament for the treatment of a skin condition in a subject, wherein the second composition is onabotulinumtoxinA, and the skin condition in the subject is acne rosacea type 2. In another aspect is provided a composition for the manufacture of a medicament for the treatment of a skin condition in a subject, wherein the second composition is onabotulinumtoxinA, and the skin condition in the subject is psoriasis. In another aspect is provided a composition for the manufacture of a medicament for the treatment of a skin condition in a subject, wherein the second composition is onabotulinumtoxinA, and the skin condition in the subject is hyperhidrosis.

In another aspect is provided a method of treating hyperhidrosis in a subject, comprising applying to the skin of the subject a first composition comprising Spongilla, and a second composition comprising one or more botulinum toxins. In another aspect is provided any of the methods disclosed herein for treating hyperhidrosis, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided any of the methods disclosed herein for treating hyperhidrosis, wherein the second composition comprises one or more botulinum toxin type selected from botulinum toxin type A. In another aspect is provided any of the methods disclosed herein for treating hyperhidrosis, wherein the botulinum toxin type A is onabotulinumtoxinA. In another aspect is provided any of the methods disclosed herein for treating hyperhidrosis, wherein the subject experiences at least a one-grade improvement in the HDSS score following treatment compared to the subject's HDSS score prior to treatment. In another aspect is provided any of the methods disclosed herein for treating hyperhidrosis, wherein the subject experiences at least a two-grade improvement in the HDSS score following treatment compared to the subject's HDSS score prior to treatment. In another aspect is provided any of the methods disclosed herein for treating hyperhidrosis, wherein the subject experiences at least a three-grade improvement in the HDSS score following treatment compared to the subject's HDSS score prior to treatment. In another aspect is provided any of the methods disclosed herein for treating hyperhidrosis, wherein the subject experiences a greater than 10% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 20%, 30%, 40%, 50%, 60%, 70%, 80%, 90%, or 95% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 30% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 40% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 50% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 60% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 70% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 80% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 90% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 95% reduction in gravimetrically-measured sweat production following treatment compared to the subject's gravimetrically-measured sweat production prior to treatment. In another aspect is provided any of the methods disclosed herein for treating hyperhidrosis, wherein the subject is treated once per month, twice per month, three times per month, four times per month, five times per month, six times per month, 7 times per month, 8 times per month, 9 times per month, 10 times per month, 11 times per month, 12 times per month, 13 times per month, 14 times per month, 15 times per month, 16 times per month, 17 times per month, 18 times per month, 19 times per month, 20 times per month, 21 times per month, 22 times per month, or 24 times per month. In another aspect is provided any of the methods disclosed herein for treating hyperhidrosis, wherein the subject is treated twice per month. In another aspect is provided any of the methods disclosed herein for treating hyperhidrosis, wherein the subject is treated three times per month. In another aspect is provided any of the methods disclosed herein for treating hyperhidrosis, wherein the subject is treated once per week. In another aspect is provided any of the methods disclosed herein for treating hyperhidrosis, wherein the subject is at least 18 years old. In another aspect is provided any of the methods disclosed herein for treating hyperhidrosis, wherein the subject is treated once per month, or once every two months, or once every three months, or once every four months, or once every five months, or once every six months, or once every seven months, or once every eight months, or once every nine months, or once every 10 months, or once every 11 months, or once every 12 months. In another aspect is provided any of the methods disclosed herein for treating hyperhidrosis, wherein the subject is treated once per month. In another aspect is provided any of the methods disclosed herein for treating hyperhidrosis, wherein the subject is treated once every two months. In another aspect is provided any of the methods disclosed herein for treating hyperhidrosis, wherein the subject is treated once every three months. In another aspect is provided any of the methods disclosed herein for treating hyperhidrosis, wherein the subject is treated once every four months. In another aspect is provided any of the methods disclosed herein for treating hyperhidrosis, wherein the subject is treated once every five months. In another aspect is provided any of the methods disclosed herein for treating hyperhidrosis, wherein the subject is treated once every six months. In another aspect is provided any of the methods disclosed herein for treating hyperhidrosis, wherein the subject is treated once every seven months. In another aspect is provided any of the methods disclosed herein for treating hyperhidrosis, wherein the subject is treated once every eight months. In another aspect is provided any of the methods disclosed herein for treating hyperhidrosis, wherein the subject is treated once every nine months. In another aspect is provided any of the methods disclosed herein for treating hyperhidrosis, wherein the subject is treated once every 10 months. In another aspect is provided any of the methods disclosed herein for treating hyperhidrosis, wherein the subject is treated once every 11 months. In another aspect is provided any of the methods disclosed herein for treating hyperhidrosis, wherein the subject is treated once every 12 months. In another aspect is provided any of the methods disclosed herein for treating hyperhidrosis, wherein the subject suffers from primary axillary hyperhidrosis.

In another aspect is provided a method of treating hyperhidrosis in a subject, comprising applying to the skin of the subject a first composition comprising Spongilla, and a second composition comprising one or more botulinum toxins. In another aspect is provided any of the methods disclosed herein for treating hyperhidrosis, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided any of the methods disclosed herein for treating hyperhidrosis, wherein the second composition comprises one or more botulinum toxin type selected from botulinum toxin type A. In another aspect is provided any of the methods disclosed herein for treating hyperhidrosis, wherein the botulinum toxin type A is onabotulinumtoxinA. In another aspect is provided any of the methods disclosed herein for treating hyperhidrosis, wherein the subject experiences at least a at least a 4-point improvement from baseline in the weekly mean Axillary Sweating Daily Diary (ASDD) item #2 score compared to the subject's ASDD score prior to treatment. In another aspect is provided any of the methods disclosed herein for treating hyperhidrosis, wherein the subject experiences at least a at least a 4-point improvement from baseline in the weekly mean Axillary Sweating Daily Diary (ASDD) item #2 score at week four following treatment compared to the subject's ASDD score prior to treatment In another aspect is provided any of the methods disclosed herein for treating hyperhidrosis, wherein the subject experiences at least a two-grade improvement in the ASDD score following treatment compared to the subject's ASDD score prior to treatment. In another aspect is provided any of the methods disclosed herein for treating hyperhidrosis, wherein the subject experiences at least a three-grade improvement in the ASDD score following treatment compared to the subject's ASDD score prior to treatment. In another aspect is provided any of the methods disclosed herein for treating hyperhidrosis, wherein the subject experiences a greater than 10% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 20%, 30%, 40%, 50%, 60%, 70%, 80%, 90%, or 95% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 30% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 40% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 50% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 60% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 70% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 80% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 90% reduction in gravimetrically-measured sweat production following treatment compared to the subject's gravimetrically-measured sweat production prior to treatment. In another aspect is provided any of the methods disclosed herein for treating hyperhidrosis, wherein the subject experiences a greater than 95% reduction in gravimetrically-measured sweat production following treatment compared to the subject's gravimetrically-measured sweat production prior to treatment. In another aspect is provided any of the methods disclosed herein for treating hyperhidrosis, wherein the subject is treated once per month, twice per month, three times per month, four times per month, five times per month, six times per month, 7 times per month, 8 times per month, 9 times per month, 10 times per month, 11 times per month, 12 times per month, 13 times per month, 14 times per month, 15 times per month, 16 times per month, 17 times per month, 18 times per month, 19 times per month, 20 times per month, 21 times per month, 22 times per month, or 24 times per month. In another aspect is provided any of the methods disclosed herein for treating hyperhidrosis, wherein the subject is treated twice per month. In another aspect is provided any of the methods disclosed herein for treating hyperhidrosis, wherein the subject is treated three times per month. In another aspect is provided any of the methods disclosed herein for treating hyperhidrosis, wherein the subject is treated once per week. In another aspect is provided any of the methods disclosed herein for treating hyperhidrosis, wherein the subject is at least 18 years old. In another aspect is provided any of the methods disclosed herein for treating hyperhidrosis, wherein the subject is treated once per month, or once every two months, or once every three months, or once every four months, or once every five months, or once every six months, or once every seven months, or once every eight months, or once every nine months, or once every 10 months, or once every 11 months, or once every 12 months. In another aspect is provided any of the methods disclosed herein for treating hyperhidrosis, wherein the subject is treated once per month. In another aspect is provided any of the methods disclosed herein for treating hyperhidrosis, wherein the subject is treated once every two months. In another aspect is provided any of the methods disclosed herein for treating hyperhidrosis, wherein the subject is treated once every three months. In another aspect is provided any of the methods disclosed herein for treating hyperhidrosis, wherein the subject is treated once every four months. In another aspect is provided any of the methods disclosed herein for treating hyperhidrosis, wherein the subject is treated once every five months. In another aspect is provided any of the methods disclosed herein for treating hyperhidrosis, wherein the subject is treated once every six months. In another aspect is provided any of the methods disclosed herein for treating hyperhidrosis, wherein the subject is treated once every seven months. In another aspect is provided any of the methods disclosed herein for treating hyperhidrosis, wherein the subject is treated once every eight months. In another aspect is provided any of the methods disclosed herein for treating hyperhidrosis, wherein the subject is treated once every nine months. In another aspect is provided any of the methods disclosed herein for treating hyperhidrosis, wherein the subject is treated once every 10 months. In another aspect is provided any of the methods disclosed herein for treating hyperhidrosis, wherein the subject is treated once every 11 months. In another aspect is provided any of the methods disclosed herein for treating hyperhidrosis, wherein the subject is treated once every 12 months. In another aspect is provided any of the methods disclosed herein for treating hyperhidrosis, wherein the subject suffers from primary axillary hyperhidrosis.

In another aspect is provided any of the methods, compositions, compositions for use, and kits disclosed herein wherein the percentage of treated subjects having gravimetric sweat production of ≤50 mg per axilla at 4 weeks following the application of the composition comprising Spongilla and the composition comprising one or more botulinum toxins is about 10% of subjects, about 15% of subjects, about 20% of subjects, about 25% of subjects, about 30% of subjects, about 35% of subjects, about 40% of subjects, about 45% of subjects, about 50% of subjects, about 55% of subjects, about 60% of subjects, about 65% of subjects, about 70% of subjects, about 75% of subjects, about 80% of subjects, about 85% of subjects, about 90% of subjects, about 95% of subjects, or about 99% of subjects. In another aspect is provided any of the methods, compositions, compositions for use, and kits, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G. In another aspect is provided any of the methods, compositions, compositions for use, and kits, wherein the second composition comprises one or more botulinum toxin type selected from botulinum toxin type A. In another aspect is provided any of the methods, compositions, compositions for use, and kits, wherein the botulinum toxin type A is onabotulinumtoxinA. In another aspect is provided any of the methods, compositions, compositions for use, and kits, wherein the subject is treated once per month, twice per month, three times per month, four times per month, five times per month, six times per month, 7 times per month, 8 times per month, 9 times per month, 10 times per month, 11 times per month, 12 times per month, 13 times per month, 14 times per month, 15 times per month, 16 times per month, 17 times per month, 18 times per month, 19 times per month, 20 times per month, 21 times per month, 22 times per month, or 24 times per month. In another aspect is provided any of the methods, compositions, compositions for use, and kits, wherein the subject is treated twice per month. In another aspect is provided any of the methods, compositions, compositions for use, and kits, wherein the subject is treated three times per month. In another aspect is provided any of the methods, compositions, compositions for use, and kits, wherein the subject is treated once per week. In another aspect is provided any of the methods, compositions, compositions for use, and kits, wherein the subject is at least 18 years old. In another aspect is provided any of the methods, compositions, compositions for use, and kits, wherein the subject is treated once per month, or once every two months, or once every three months, or once every four months, or once every five months, or once every six months, or once every seven months, or once every eight months, or once every nine months, or once every 10 months, or once every 11 months, or once every 12 months. In another aspect is provided any of the methods, compositions, compositions for use, and kits, wherein the subject is treated once per month. In another aspect is provided any of the methods, compositions, compositions for use, and kits, wherein the subject is treated once every two months. In another aspect is provided any of the methods, compositions, compositions for use, and kits, wherein the subject is treated once every three months. In another aspect is provided any of the methods, compositions, compositions for use, and kits, wherein the subject is treated once every four months. In another aspect is provided any of methods, compositions, compositions for use, and kits, wherein the subject is treated once every five months. In another aspect is provided any of the methods, compositions, compositions for use, and kits, wherein the subject is treated once every six months. In another aspect is provided any of the methods, compositions, compositions for use, and kits, wherein the subject is treated once every seven months. In another aspect is provided any of methods, compositions, compositions for use, and kits, wherein the subject is treated once every eight months. In another aspect is provided any of methods, compositions, compositions for use, and kits, wherein the subject is treated once every nine months. In another aspect is provided any of the methods, compositions, compositions for use, and kits, wherein the subject is treated once every 10 months. In another aspect is provided any of the methods, compositions, compositions for use, and kits, wherein the subject is treated once every 11 months. In another aspect is provided any of the methods, compositions, compositions for use, and kits, wherein the subject is treated once every 12 months. In another aspect is provided any of the methods, compositions, compositions for use, and kits, wherein the subject suffers from primary axillary hyperhidrosis.

In another aspect is provided any of the methods disclosed herein for treating hyperhidrosis, wherein the first composition comprises from about 0.25 grams to about 10 grams Spongilla. In another aspect is provided any of the methods disclosed herein for treating hyperhidrosis, wherein the composition comprises Spongilla and hydrogen peroxide solution. In another aspect is provided any of the methods disclosed herein for treating hyperhidrosis, wherein the hydrogen peroxide solution comprises about 3% hydrogen peroxide. In another aspect is provided any of the methods disclosed herein for treating hyperhidrosis, wherein the first composition comprises about 2 grams of Spongilla and about 6 mL of 3% hydrogen peroxide or about 6 mL of saline.

In another aspect is provided any of the methods disclosed herein, wherein the first composition comprises Spongilla in the form of a powder. Materials comprising Spongilla may be prepared in powdered form having particles of substantially the same size, using techniques known to those having ordinary skill in the art, such as grinding and sieving. In another aspect is provided any of the methods disclosed herein, wherein the Spongilla is in the form of a powder comprising particles that are substantially uniform in size. In another aspect is provided any of the methods disclosed herein, wherein not less than about 50% of the particles comprising the Spongilla powder pass through a US 70-mesh screen. In another aspect is provided any of the methods disclosed herein, wherein not less than about 60%, or about 70%, or about 75%, or about 80%, or about 85%, or about 90%, or about 95%, or about 96%, or about 97%, or about 98%, or about 99% of the particles comprising the Spongilla powder pass through a US 70-mesh screen. In another aspect is provided any of the methods disclosed herein, wherein not less than about 95%, or about 96%, or about 97%, or about 98%, or about 99% of the particles comprising the Spongilla powder pass through a US 70-mesh screen. In another aspect is provided any of the methods disclosed herein, wherein not less than about 95% of the particles comprising the Spongilla powder pass through a US 70-mesh screen. In another aspect is provided any of the methods disclosed herein, wherein not less than about 96% of the particles comprising the Spongilla powder pass through a US 70-mesh screen. In another aspect is provided any of the methods disclosed herein, wherein not less than about 97% of the particles comprising the Spongilla powder pass through a US 70-mesh screen. In another aspect is provided any of the methods disclosed herein, wherein not less than about 98% of the particles comprising the Spongilla powder pass through a US 70-mesh screen. In another aspect is provided any of the methods disclosed herein, wherein not less than about 99% of the particles comprising the Spongilla powder pass through a US 70-mesh screen. The particles of Spongilla may be manufactured or produced from Spongilla materials that are harvested by procedures known to those of ordinary skill in the art, such as determining the appropriate harvest period, removal of foreign materials, drying, milling and grinding using equipment known to those of ordinary skill in the art.

In another aspect is provided any of the methods disclosed herein, wherein the particles comprising the Spongilla powder have an average length of from about 50 μm to about 500 μm. In another aspect is provided any of the methods disclosed herein, wherein the particles comprising the Spongilla powder have an average length of from about 50 μm to about 400 μm, or from about 50 μm to about 350 μm, or from about 50 μm to about 300 μm, or from about 50 μm to about 250 μm, or from about 50 μm to about 200 μm, or from about 75 μm to about 500 μm, or from about 75 μm to about 450 μm, or from about 80 μm to about 450 μm, or from about 80 μm to about 400 μm, or from about 85 μm to about 450 μm, or from about 85 μm to about 400 μm, or from about 90 μm to about 450 μm, or from about 90 μm to about 400 μm, or from about 90 μm to about 350 μm, or from about 100 μm to about 450 μm, or from about 100 μm to about 400 μm, or from about 100 μm to about 350 μm, or from about 100 μm to about 300 μm, or from about 100 μm to about 250 μm, or from about 100 μm to about 200 μm, or from about 150 μm to about 500 μm, or from about 150 μm to about 450 μm, or from about 150 μm to about 400 μm, or from about 150 μm to about 350 μm, or from about 150 μm to about 350 μm, or from about 150 μm to about 300 μm, or from about 150 μm to about 250 μm, or from about 150 μm to about 200 μm, or from about 175 μm to about 450 μm, or from about 175 μm to about 400 μm, or from about 175 μm to about 350 μm, or from about 175 μm to about 300 μm, or from about 175 μm to about 250 μm, or from about 175 μm to about 200 μm. In another aspect is provided any of the methods disclosed herein, wherein the particles comprising the Spongilla powder have an average length of about 50 μm, or about 75 μm, or about 80 μm, or about 85 μm, or about 90 μm, or about 100 μm, or about 125 μm, or about 150 μm, or about 175 μm, or about 200 μm, or about 225 μm, or about 250 μm, or about 300 μm, or about 350 μm, or about 400 μm, or about 450 μm, or about 500 μm. In another aspect is provided any of the methods disclosed herein, wherein the particles comprising the Spongilla powder have an average length of about 200 μm. The particles comprising the Spongilla powder may be manufactured or produced from Spongilla materials that are harvested by procedures known to those of ordinary skill in the art, such as milling and grinding using equipment known to those of ordinary skill in the art. The average length of particles comprising the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder.

In another aspect is provided any of the methods disclosed herein, wherein the particles comprising the Spongilla powder have an average diameter of from about 5 μm to about 50 μm. In another aspect is provided any of the methods disclosed herein, wherein the particles comprising the Spongilla powder have an average diameter of from about 5 μm to about 45 μm, or from about 5 μm to about 40 μm, from about 5 μm to about 35 μm, from about 5 μm to about 30 μm, from about 5 μm to about 25 μm, from about 5 μm to about 20 μm, from about 10 μm to about 50 μm, from about 10 μm to about 45 μm, from about 10 μm to about 40 μm, from about 10 μm to about 35 μm, from about 10 μm to about 30 μm, from about 10 μm to about 25 μm, from about 10 μm to about 20 μm. In another aspect is provided any of the methods disclosed herein, wherein the particles comprising the Spongilla powder have an average diameter of about 5 μm, or about 10 μm, or about 15 μm, or about 20 μm, or about 25 μm, or about 30 μm, or about 35 μm, or about 40 μm, or about 45 μm, or about 50 μm. The particles comprising the Spongilla powder may be manufactured or produced from Spongilla materials that are harvested by procedures known to those of ordinary skill in the art, such as milling and grinding using equipment known to those of ordinary skill in the art. The average diameter of particles comprising the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder

In another aspect is provided any of the methods disclosed herein, wherein the particles comprising the Spongilla powder have an aspect ratio of from about 1 to about 100. In another aspect is provided any of the methods disclosed herein, wherein the particles comprising the Spongilla powder have an aspect ratio of from about 1 to about 75, or from about 1 to about 50, or from about 1 to about 25, or from about 1 to about 20, or from about 1 to about 15, or from about 5 to about 100, or from about 5 to about 75, or from about 5 to about 50, or from about 5 to about 40, or from about 5 to about 35, or from about 5 to about 30, or from about 5 to about 25, or from about 5 to about 20, or from about 5 to about 15, or from about 7 to about 50, or from about 7 to about 45, or from about 7 to about 40, or from about 7 to about 35, or from about 7 to about 30, or from about 7 to about 25, or from about 10 to about 50, or from about 10 to about 45, or from about 10 to about 40, or from about 10 to about 35, or from about 10 to about 30, or from about 10 to about 25, from about 10 to about 20, or from about 10 to about 15. In another aspect is provided any of the methods disclosed herein, wherein the particles comprising the Spongilla powder have an aspect ratio of about 5, or about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 35, or about 40, or about 45, or about 50, or about 75, or about 100. The particles comprising the Spongilla powder may be manufactured or produced from Spongilla materials that are harvested by procedures known to those of ordinary skill in the art, such as milling and grinding using equipment known to those of ordinary skill in the art. The aspect ratio of particles comprising the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder

Materials comprising Spongilla may be processed and dried, using techniques known to those having ordinary skill in the art, such as the use of drying ovens, to provide materials having a desired residual moisture content. In another aspect is provided any of the methods disclosed herein, wherein the first composition comprising Spongilla has a residual moisture content of not more than about 20%. In another aspect is provided any of the methods disclosed herein, wherein the first composition has a residual moisture content of not more than about 15%, or not more than about 10%, or not more than about 9%, or not more than about 8%, or not more than about 7%, or not more than about 6%, or not more than about 5%, or not more than about 4%, or not more than about 3%, or not more than about 2%, or not more than 1%. In another aspect is provided any of the methods disclosed herein, wherein the first composition has a residual moisture content of not more than about 5%. In another aspect is provided any of the methods disclosed herein, wherein the first composition has a residual moisture content of not more than about 4%. In another aspect is provided any of the methods disclosed herein, wherein the first composition has a residual moisture content of not more than about 3%. In another aspect is provided any of the methods disclosed herein, wherein the first composition has a residual moisture content of not more than about 2%. In another aspect is provided any of the methods disclosed herein, wherein the first composition has a residual moisture content of not more than about 1%. The moisture content of the Spongilla materials can be reduced by heating the raw Spongilla materials using methods known to those of ordinary skill in the art, such as by open-air drying, or by use of a conventional oven dryer or a vacuum dryer, using equipment known to those of ordinary skill in the art. For example, raw Spongilla materials may be placed into a tray and heated in a drying oven at a temperature range from about 30° C. to about 200° C., for example to about 70° C., for a period of time necessary to reduce the residual moisture content to the desired level. The level of residual moisture of the materials may be measured using methods known to those of ordinary skill in the art such as those described in the United States Pharmacopeia methods USP <731> (Loss on Drying) and USP <921> (Water Determination).

Materials comprising Spongilla may be treated in order to reduce the bioburden, such as aerobic and anaerobic microbes, yeast and mold, Coliform bacteria, Salmonella, Pseudomonas aeruginosa, and Staphylococcus aureus, of the materials prior to their packaging and use, such as by use of heat treatment or irradiation, such as the use of gamma irradiation.

In another aspect is provided any of the methods disclosed herein, wherein the first composition has a combined aerobic and anaerobic microbial content of not more than about 25×10⁴ colony-forming units per gram (CFU/g). In another aspect is provided any of the methods disclosed herein, wherein the first composition has a combined aerobic and anaerobic microbial content of not more than about 10×10⁴ CFU/g, or not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods disclosed herein, wherein the first composition has a combined aerobic and anaerobic microbial content of not more than about 1,000 CFU/g. In another aspect is provided any of the methods disclosed herein, wherein the first composition has a combined aerobic and anaerobic microbial content of not more than about 750 CFU/g. In another aspect is provided any of the methods disclosed herein, wherein the first composition has a combined aerobic and anaerobic microbial content of not more than about 500 CFU/g. In another aspect is provided any of the methods disclosed herein, wherein the first composition has a combined aerobic and anaerobic microbial content of not more than about 250 CFU/g. In another aspect is provided any of the methods disclosed herein, wherein the first composition has a combined aerobic and anaerobic microbial content of not more than about 200 CFU/g. In another aspect is provided any of the methods disclosed herein, wherein the first composition has a combined aerobic and anaerobic microbial content of not more than about 150 CFU/g. In another aspect is provided any of the methods disclosed herein, wherein the first composition has a combined aerobic and anaerobic microbial content of not more than about 100 CFU/g. In another aspect is provided any of the methods disclosed herein, wherein the first composition has a combined aerobic and anaerobic microbial content of not more than about 75 CFU/g. In another aspect is provided any of the methods disclosed herein, wherein the first composition has a combined aerobic and anaerobic microbial content of not more than about 50 CFU/g. In another aspect is provided any of the methods disclosed herein, wherein the first composition has a combined aerobic and anaerobic microbial content of not more than about 25 CFU/g. In another aspect is provided any of the methods disclosed herein, wherein the first composition has a combined aerobic and anaerobic microbial content of not more than about 20 CFU/g. In another aspect is provided any of the methods disclosed herein, wherein the first composition has a combined aerobic and anaerobic microbial content of not more than about 10 CFU/g. In another aspect is provided any of the methods disclosed herein, wherein the first composition has a combined aerobic and anaerobic microbial content of not more than about 1 CFU/g. The combined aerobic and anaerobic microbial content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The combined aerobic and anaerobic microbial content of the Spongilla materials may be measured by methods known to those of ordinary skill in the art, such as those described in the United States Pharmacopeia method USP <61> (Microbial Enumeration Tests).

In another aspect is provided any of the methods disclosed herein, wherein the first composition comprising Spongilla has a combined yeast and mold content of not more than about 25×10⁴ colony-forming units per gram (CFU/g). In another aspect is provided any of the methods disclosed herein, wherein the first composition has a combined yeast and mold content of not more than about or not more than about 5×10⁴ CFU/g, or not more than about 1×10⁴ CFU/g, or not more than about 5×10⁴ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods disclosed herein, wherein the first composition has a combined yeast and mold content of not more than about 1,000 CFU/g. In another aspect is provided any of the methods disclosed herein, wherein the first composition has a combined yeast and mold content of not more than about 750 CFU/g. In another aspect is provided any of the methods disclosed herein, wherein the first composition has a combined yeast and mold content of not more than about 500 CFU/g. In another aspect is provided any of the methods disclosed herein, wherein the first composition has a combined yeast and mold content of not more than about 250 CFU/g. In another aspect is provided any of the methods disclosed herein, wherein the first composition has a combined yeast and mold content of not more than about 200 CFU/g. In another aspect is provided any of the methods disclosed herein, wherein the first composition has a combined yeast and mold content of not more than about 150 CFU/g. In another aspect is provided any of the methods disclosed herein, wherein the first composition has a combined yeast and mold content of not more than about 100 CFU/g. In another aspect is provided any of the methods disclosed herein, wherein the first composition has a combined yeast and mold content of not more than about 75 CFU/g. In another aspect is provided any of the methods disclosed herein, wherein the first composition has a combined yeast and mold content of not more than about 50 CFU/g. In another aspect is provided any of the methods disclosed herein, wherein the first composition has a combined yeast and mold content of not more than about 25 CFU/g. In another aspect is provided any of the methods disclosed herein, wherein the first composition has a combined yeast and mold content of not more than about 20 CFU/g. In another aspect is provided any of the methods disclosed herein, wherein the first composition has a combined yeast and mold content of not more than about 10 CFU/g. In another aspect is provided any of the methods disclosed herein, wherein the first composition has a combined yeast and mold content of not more than about 1 CFU/g. The combined yeast and mold content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The combined yeast and mold content of the Spongilla materials may be measured by methods known to those of ordinary skill in the art, such as those described in the United States Pharmacopeia method USP <61> (Microbial Enumeration Tests).

In another aspect is provided any of the methods disclosed herein, wherein the amount of Coliform bacteria in the first composition is not more than about 25×10⁴ colony-forming units per gram (CFU/g). In another aspect is provided any of the methods disclosed herein, wherein the amount of Coliform bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods disclosed herein, wherein the first composition has a Coliform bacteria content of not more than about 1,000 CFU/g. In another aspect is provided any of the methods disclosed herein, wherein the first composition has a Coliform bacteria content of not more than about 750 CFU/g. In another aspect is provided any of the methods disclosed herein, wherein the first composition has a Coliform bacteria content of not more than about 500 CFU/g. In another aspect is provided any of the methods disclosed herein, wherein the first composition has a Coliform bacteria content of not more than about 250 CFU/g. In another aspect is provided any of the methods disclosed herein, wherein the first composition has a Coliform bacteria content of not more than about 200 CFU/g. In another aspect is provided any of the methods disclosed herein, wherein the first composition has a Coliform bacteria content of not more than about 150 CFU/g. In another aspect is provided any of the methods disclosed herein, wherein the first composition has a Coliform bacteria content of not more than about 100 CFU/g. In another aspect is provided any of the methods disclosed herein, wherein the first composition has a Coliform bacteria content of not more than about 75 CFU/g. In another aspect is provided any of the methods disclosed herein, wherein the first composition has a Coliform bacteria content of not more than about 50 CFU/g. In another aspect is provided any of the methods disclosed herein, wherein the first composition has a Coliform bacteria content of not more than about 25 CFU/g. In another aspect is provided any of the methods disclosed herein, wherein the first composition has a Coliform bacteria content of not more than about 20 CFU/g. In another aspect is provided any of the methods disclosed herein, wherein the first composition has a Coliform bacteria content of not more than about 10 CFU/g. In another aspect is provided any of the methods disclosed herein, wherein the first composition has a Coliform bacteria content of not more than about 1 CFU/g. In another aspect is provided any of the methods disclosed herein, wherein the first composition has no detectable Coliform bacterial content. The Coliform bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Coliform bacteria content of the Spongilla materials may be measured by methods known to those of ordinary skill in the art, such as those described in the United States Pharmacopeia method USP <62> (Tests for Specified Microorganisms).

In another aspect is provided any of the methods disclosed herein, wherein the amount of Salmonella in the first composition is not more than about 25×10⁴ colony-forming units per gram (CFU/g). In another aspect is provided any of the methods disclosed herein, wherein the amount of Salmonella in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods disclosed herein, wherein the first composition has a Salmonella content of not more than about 1,000 CFU/g. In another aspect is provided any of the methods disclosed herein, wherein the first composition has a Salmonella content of not more than about 750 CFU/g. In another aspect is provided any of the methods disclosed herein, wherein the first composition has a Salmonella content of not more than about 500 CFU/g. In another aspect is provided any of the methods disclosed herein, wherein the first composition has a Salmonella content of not more than about 250 CFU/g. In another aspect is provided any of the methods disclosed herein, wherein the first composition has a Salmonella content of not more than about 200 CFU/g. In another aspect is provided any of the methods disclosed herein, wherein the first composition has a Salmonella content of not more than about 150 CFU/g. In another aspect is provided any of the methods disclosed herein, wherein the first composition has a Salmonella content of not more than about 100 CFU/g. In another aspect is provided any of the methods disclosed herein, wherein the first composition has a Salmonella content of not more than about 75 CFU/g. In another aspect is provided any of the methods disclosed herein, wherein the first composition has a Salmonella content of not more than about 50 CFU/g. In another aspect is provided any of the methods disclosed herein, wherein the first composition has a Salmonella content of not more than about 25 CFU/g. In another aspect is provided any of the methods disclosed herein, wherein the first composition has a Salmonella content of not more than about 20 CFU/g. In another aspect is provided any of the methods disclosed herein, wherein the first composition has a Salmonella content of not more than about 10 CFU/g. In another aspect is provided any of the methods disclosed herein, wherein the first composition has a Salmonella content of not more than about 1 CFU/g. In another aspect is provided any of the methods disclosed herein, wherein the first composition has no detectable Salmonella content. The Salmonella content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Salmonella content of the Spongilla materials may be measured by methods known to those of ordinary skill in the art, such as those described in the United States Pharmacopeia method USP <62> (Tests for Specified Microorganisms).

In another aspect is provided any of the methods disclosed herein, wherein the amount of Pseudomonas aeruginosa bacteria in the first composition is not more than about 25×10⁴ colony-forming units per gram (CFU/g). In another aspect is provided any of the methods disclosed herein, wherein the amount of Pseudomonas aeruginosa bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods disclosed herein, wherein the first composition has a Pseudomonas aeruginosa bacteria content of not more than about 1,000 CFU/g. In another aspect is provided any of the methods disclosed herein, wherein the first composition has a Pseudomonas aeruginosa bacteria content of not more than about 750 CFU/g. In another aspect is provided any of the methods disclosed herein, wherein the first composition has a Pseudomonas aeruginosa bacteria content of not more than about 500 CFU/g. In another aspect is provided any of the methods disclosed herein, wherein the first composition has a Pseudomonas aeruginosa bacteria content of not more than about 250 CFU/g. In another aspect is provided any of the methods disclosed herein, wherein the first composition has a Pseudomonas aeruginosa bacteria content of not more than about 200 CFU/g. In another aspect is provided any of the methods disclosed herein, wherein the first composition has a Pseudomonas aeruginosa bacteria content of not more than about 150 CFU/g. In another aspect is provided any of the methods disclosed herein, wherein the first composition has a Pseudomonas aeruginosa bacteria content of not more than about 100 CFU/g. In another aspect is provided any of the methods disclosed herein, wherein the first composition has a Pseudomonas aeruginosa bacteria content of not more than about 75 CFU/g. In another aspect is provided any of the methods disclosed herein, wherein the first composition has a Pseudomonas aeruginosa bacteria content of not more than about 50 CFU/g. In another aspect is provided any of the methods disclosed herein, wherein the first composition has a Pseudomonas aeruginosa bacteria content of not more than about 25 CFU/g. In another aspect is provided any of the methods disclosed herein, wherein the first composition has a Pseudomonas aeruginosa bacteria content of not more than about 20 CFU/g. In another aspect is provided any of the methods disclosed herein, wherein the first composition has a Pseudomonas aeruginosa bacteria content of not more than about 10 CFU/g. In another aspect is provided any of the methods disclosed herein, wherein the first composition has a Pseudomonas aeruginosa bacteria content of not more than about 1 CFU/g. In another aspect is provided any of the methods disclosed herein, wherein the first composition has no detectable Pseudomonas aeruginosa bacteria content. The Pseudomonas aeruginosa bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Pseudomonas aeruginosa bacteria content of the Spongilla materials may be measured by methods known to those of ordinary skill in the art, such as those described in the United States Pharmacopeia method USP <62> (Tests for Specified Microorganisms).

In another aspect is provided any of the methods disclosed herein, wherein the amount of Staphylococcus aureus bacteria in the first composition is not more than about 25×10⁴ colony-forming units per gram (CFU/g). In another aspect is provided any of the methods disclosed herein, wherein the amount of Staphylococcus aureus bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods disclosed herein, wherein the first composition has a Staphylococcus aureus bacteria content of not more than about 1,000 CFU/g. In another aspect is provided any of the methods disclosed herein, wherein the first composition has a Staphylococcus aureus bacteria content of not more than about 750 CFU/g. In another aspect is provided any of the methods disclosed herein, wherein the first composition has a Staphylococcus aureus bacteria content of not more than about 500 CFU/g. In another aspect is provided any of the methods disclosed herein, wherein the first composition has a Staphylococcus aureus bacteria content of not more than about 250 CFU/g. In another aspect is provided any of the methods disclosed herein, wherein the first composition has a Staphylococcus aureus bacteria content of not more than about 200 CFU/g. In another aspect is provided any of the methods disclosed herein, wherein the first composition has a Staphylococcus aureus bacteria content of not more than about 150 CFU/g. In another aspect is provided any of the methods disclosed herein, wherein the first composition has a Staphylococcus aureus bacteria content of not more than about 100 CFU/g. In another aspect is provided any of the methods disclosed herein, wherein the first composition has a Staphylococcus aureus bacteria content of not more than about 75 CFU/g. In another aspect is provided any of the methods disclosed herein, wherein the first composition has a Staphylococcus aureus bacteria content of not more than about 50 CFU/g. In another aspect is provided any of the methods disclosed herein, wherein the first composition has a Staphylococcus aureus bacteria content of not more than about 25 CFU/g. In another aspect is provided any of the methods disclosed herein, wherein the first composition has a Staphylococcus aureus bacteria content of not more than about 20 CFU/g. In another aspect is provided any of the methods disclosed herein, wherein the first composition has a Staphylococcus aureus bacteria content of not more than about 10 CFU/g. In another aspect is provided any of the methods disclosed herein, wherein the first composition has a Staphylococcus aureus bacteria content of not more than about 1 CFU/g. In another aspect is provided any of the methods disclosed herein, wherein the first composition has no detectable Staphylococcus aureus bacteria content. The Staphylococcus aureus bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Staphylococcus aureus bacteria content of the Spongilla materials may be measured by methods known to those of ordinary skill in the art, such as those described in the United States Pharmacopeia method USP <62> (Tests for Specified Microorganisms).

In another aspect is provided any of the methods disclosed herein, wherein the first composition is packaged prior to use. In another aspect is provided any of the methods disclosed herein, wherein the first composition is prepared by heating to at least about 70° C. prior to being packaged in order to reduce the bioburden. In another aspect is provided any of the methods disclosed herein, wherein the first composition is prepared by heating to at least about 50° C., or at least about 60° C., or at least about 75° C., or at least about 80° C., or at least about 85° C., or at least about 90° C., or at least about 100° C., or at least about 110° C., or at least about 115° C., or at least about 120° C., or at least about 125° C., or at least about 130° C., or at least about 135° C., or at least about 140° C., or at least about 150° C., or at least about 160° C., or at least about 170° C., or at least about 180° C., or at least about 190° C., or at least about 200° C. prior to being packaged.

In another aspect is provided any of the methods disclosed herein, wherein the first composition comprising Spongilla is heated to at least about 70° C. for at least about 5 minutes prior being packaged. In another aspect is provided any of the methods disclosed herein, wherein the first composition is heated to at least about 70° C. for at least about 10 minutes, or at least about 15 minutes, or at least about 20 minutes, or at least about 25 minutes, or at least about 30 minutes, or at least about 35 minutes, or at least about 40 minutes, or at least about 45 minutes, or at least about 50 minutes, or at least about 55 minutes, or at least about 60 minutes, or at least about 75 minutes, or at least about 90 minutes, or at least about 120 minutes, or at least about 180 minutes, or at least about 4 hours, or at least about 5 hours, or at least about 6 hours, or at least about 7 hours, or at least about 8 hours, or at least about 9 hours, or at least about 10 hours, or at least about 11 hours, or at least about 12 hours, or at least about 24 hours prior being packaged.

In another aspect is provided any of the methods disclosed herein, wherein the first composition comprising Spongilla is prepared by treating with ionizing radiation, such as gamma radiation, prior to being packaged or after packaging. For example, gamma irradiation may be performed on the raw Spongilla material prior to grinding to reduce the particle size, following grinding to reduce the particle size, the materials packaged in bulk and or the materials following packaging in unit dose containers. The materials may be treated with ionizing radiation, such as gamma radiation, using methods and equipment known to those of ordinary skill in the art, such as gamma irradiators or electron beam irradiators. In another aspect is provided any of the methods disclosed herein, wherein the first composition is prepared by treating with ionizing radiation, such as gamma radiation, to deliver an absorbed radiation dose between about 1 kGy and about 50 kGy prior to being packaged. In another aspect is provided any of the methods disclosed herein, wherein the first composition is prepared by treating with ionizing radiation, such as gamma radiation, to deliver an absorbed radiation dose between about 1 kGy and about 45 kGy, or between about 1 kGy and about 40 kGy, between about 1 kGy and about 35 kGy, between about 1 kGy and about 30 kGy, or between about 1 kGy and about 25 kGy or between about 5 kGy and about 50 kGy, or between about 5 kGy and about 45 kGy, or between about 5 kGy and about 40 kGy, or between about 5 kGy and about 35 kGy, or between about 5 kGy and about 30 kGy, or between about 5 kGy and about 25 kGy, or between about 10 kGy and about 50 kGy, or between about 10 kGy and about 45 kGy, or between about 10 kGy and about 40 kGy, or between about 10 kGy and about 35 kGy, or between about 10 kGy and about 30 kGy, or between about 10 kGy and about 25 kGy, or between about 15 kGy and about 50 kGy, or between about 15 kGy and about 45 kGy, or between about 15 kGy and about 40 kGy, or between about 15 kGy and about 35 kGy, or between about 15 kGy and about 30 kGy, or between about 15 kGy and about 25 kGy. In another aspect is provided any of the methods disclosed herein, wherein the first composition is prepared by treating with ionizing radiation, such as gamma radiation, to deliver an absorbed radiation dose of about 1 kGy, or about 5 kGy, or about 10 kGy, 11 kGy, or about 12 kGy, or about 13 kGy, or about 14 kGy, or about 15 kGy, or about 16 kGy, or about 17 kGy, or about 18 kGy, or about 19 kGy, or about 20 kGy, or about 21 kGy, or about 22 kGy, or about 23 kGy, or about 24 kGy, or about 25 kGy, or about 26 kGy, or about 27 kGy, or about 28 kGy, or about 29 kGy, or about 30 kGy, or about 31 kGy, or about 32 kGy, or about 33 kGy, or about 34 kGy, or about 35 kGy, or about 36 kGy, or about 37 kGy, or about 38 kGy, or about 39 kGy, or about 40 kGy, or about 41 kGy, or about 42 kGy, or about 43 kGy, or about 44 kGy, or about 45 kGy, or about 46 kGy, or about 47 kGy, or about 48 kGy, or about 49 kGy, or about 50 kGy.

In another aspect is provided any of the methods disclosed herein, wherein the first composition is applied to the skin of the subject in the form of a paste. In another aspect is provided any of the methods disclosed herein, wherein the paste further comprises water or saline. In another aspect is provided any of the methods disclosed herein, wherein the paste is prepared by mixing a composition comprising Spongilla and an aqueous solution comprising hydrogen peroxide. In another aspect is provided any of the methods disclosed herein, wherein the hydrogen peroxide is at a concentration of from about 0.1% w/w to about 50% w/w, or from about 0.1% w/w to about 45% w/w, or from about 0.1% w/w to about 40% w/w, or from about 0.1% w/w to about 35% w/w, or from about 0.1% w/w to about 30% w/w, or from about 0.1% w/w to about 25% w/w, or from about 0.1% w/w to about 20% w/w, or from about 0.1% w/w to about 15% w/w, or from about 0.1% w/w to about 10% w/w, or from about 0.1% w/w to about 9% w/w, or from about 0.1% w/w to about 8% w/w, or from about 0.1% w/w to about 7% w/w, or from about 0.1% w/w to about 6% w/w, or from about 0.1% w/w to about 5% w/w, or from about 0.1% w/w to about 4% w/w, or from about 0.1% w/w to about 3% w/w, or from about 0.1% w/w to about 2% w/w, or from about 0.1% w/w to about 1% w/w, or from about 0.5% w/w to about 45% w/w, or from about 1% w/w to about 45% w/w, or from about 1% w/w to about 40% w/w, or from about 1% w/w to about 35% w/w, or from about 1% w/w to about 30% w/w, or from about 1% w/w to about 25% w/w, or from about 1% w/w to about 20% w/w, or from about 1% w/w to about 15% w/w, or from about 1% w/w to about 10% w/w, or from about 1% w/w to about 9% w/w, or from about 1% w/w to about 8% w/w, or from about 1% w/w to about 7% w/w, or from about 1% w/w to about 6% w/w, or from about 1% w/w to about 5% w/w, or from about 1% w/w to about 4% w/w, or from about 1% w/w to about 3% w/w, or from about 1% w/w to about 2% w/w, or from about 2% w/w to about 45% w/w, or from about 2% w/w to about 40% w/w, or from about 2% w/w to about 35% w/w, or from about 2% w/w to about 30% w/w, or from about 2% w/w to about 25% w/w, or from about 2% w/w to about 20% w/w, or from about 2% w/w to about 15% w/w, or from about 2% w/w to about 10% w/w, or from about 2% w/w to about 9% w/w, or from about 2% w/w to about 8% w/w, or from about 1% w/w to about 7% w/w, or from about 2% w/w to about 6% w/w, or from about 2% w/w to about 5% w/w, or from about 2% w/w to about 4% w/w, or from about 2% w/w to about 3% w/w. In another aspect is provided any of the methods disclosed herein, wherein the hydrogen peroxide is at a concentration of about 0.1% w/w, or about 0.5% w/w, or about 1% w/w, or about 2% w/w, or about 3% w/w, or about 4% w/w, or about 5% w/w, or about 6% w/w, or about 7% w/w, or about 8% w/w, or about 9% w/w, or about 10% w/w, or about 15% w/w, or about 20% w/w, or about 25% w/w, or about 30% w/w, or about 35% w/w, or about 40% w/w, or about 45% w/w, or about 50% w/w. In another aspect is provided any of the methods disclosed herein, wherein the hydrogen peroxide is at a concentration of about 3% w/w. Aqueous hydrogen peroxide solutions that may be useful in treating skin conditions in a subject as disclosed herein are commercially available or may be prepared by methods known to those of ordinary skill in the art.

In another aspect is provided any of the methods disclosed herein, wherein the first composition and/or the second composition may be used in combination with a gel or cream, which gel or cream may or may not further comprise hydrogen peroxide. In another aspect is provided any of the methods disclosed herein, wherein the first composition and/or the second composition may be used in combination with a gel or cream, which gel or cream does not further comprise hydrogen peroxide. In another aspect is provided any of the methods disclosed herein, wherein the first composition and/or the second composition may be used in combination with a gel or cream, which gel or cream further comprises hydrogen peroxide. Such gels or creams are generally commercially available any may contain from about 0.5% w/w to about 50% w/w hydrogen peroxide. For example, a gel containing about 1% w/w, or about 2% w/w, or about 3% w/w, or about 4% w/w, or about 5% w/w, or about 6% w/w, or about 7% w/w, or about 8% w/w, or about 9% w/w, or about 10% w/w, or about 15% w/w, or about 20% w/w, or about 25% w/w, or about 30% w/w, or about 40% w/w, or about 45% w/w, or about 50% w/w hydrogen peroxide may be used in any of the methods disclosed herein in combination with the first composition and the second composition.

In another aspect is provided any of the methods disclosed herein, wherein the method further comprises applying a third composition to the skin of the subject. In another aspect is provided any of the methods disclosed herein, wherein the third composition comprises hydrogen peroxide. In another aspect is provided any of the methods disclosed herein, wherein the hydrogen peroxide is at a w/w concentration of about 3%. In another aspect is provided any of the methods disclosed herein, wherein the hydrogen peroxide is at a w/w concentration of about 0.1% w/w to about 50% w/w, or from about 0.1% w/w to about 45% w/w, or from about 0.1% w/w to about 40% w/w, or from about 0.1% w/w to about 35% w/w, or from about 0.1% w/w to about 30% w/w, or from about 0.1% w/w to about 25% w/w, or from about 0.1% w/w to about 20% w/w, or from about 0.1% w/w to about 15% w/w, or from about 0.1% w/w to about 10% w/w, or from about 0.1% w/w to about 9% w/w, or from about 0.1% w/w to about 8% w/w, or from about 0.1% w/w to about 7% w/w, or from about 0.1% w/w to about 6% w/w, or from about 0.1% w/w to about 5% w/w, or from about 0.1% w/w to about 4% w/w, or from about 0.1% w/w to about 3% w/w, or from about 0.1% w/w to about 2% w/w, or from about 0.1% w/w to about 1% w/w, or from about 0.5% w/w to about 45% w/w, or from about 1% w/w to about 45% w/w, or from about 1% w/w to about 40% w/w, or from about 1% w/w to about 35% w/w, or from about 1% w/w to about 30% w/w, or from about 1% w/w to about 25% w/w, or from about 1% w/w to about 20% w/w, or from about 1% w/w to about 15% w/w, or from about 1% w/w to about 10% w/w, or from about 1% w/w to about 9% w/w, or from about 1% w/w to about 8% w/w, or from about 1% w/w to about 7% w/w, or from about 1% w/w to about 6% w/w, or from about 1% w/w to about 5% w/w, or from about 1% w/w to about 4% w/w, or from about 1% w/w to about 3% w/w, or from about 1% w/w to about 2% w/w, or from about 2% w/w to about 45% w/w, or from about 2% w/w to about 40% w/w, or from about 2% w/w to about 35% w/w, or from about 2% w/w to about 30% w/w, or from about 2% w/w to about 25% w/w, or from about 2% w/w to about 20% w/w, or from about 2% w/w to about 15% w/w, or from about 2% w/w to about 10% w/w, or from about 2% w/w to about 9% w/w, or from about 2% w/w to about 8% w/w, or from about 1% w/w to about 7% w/w, or from about 2% w/w to about 6% w/w, or from about 2% w/w to about 5% w/w, or from about 2% w/w to about 4% w/w, or from about 2% w/w to about 3% w/w. In another aspect is provided any of the methods disclosed herein, wherein the hydrogen peroxide is at a concentration of about 0.1% w/w, or about 0.5% w/w, or about 1% w/w, or about 2% w/w, or about 3% w/w, or about 4% w/w, or about 5% w/w, or about 6% w/w, or about 7% w/w, or about 8% w/w, or about 9% w/w, or about 10% w/w, or about 15% w/w, or about 20% w/w, or about 25% w/w, or about 30% w/w, or about 35% w/w, or about 40% w/w, or about 45% w/w, or about 50% w/w. In another aspect is provided any of the methods disclosed herein, wherein the hydrogen peroxide is at a concentration of about 3% w/w. Aqueous hydrogen peroxide solutions that may be useful in treating skin conditions in a subject as disclosed herein are commercially available or may be prepared by methods known to those of ordinary skill in the art.

In another aspect is provided any of the methods disclosed herein, wherein the Spongilla is Spongilla lacustris.

The presence of botulinum toxin in vivo can be determined by measuring the presence of the proteolytic cleavage products derived from various SNARE proteins (Soluble NSF (N-ethylmaleimide-sensitive factor) Attachment Protein) Receptor). It known to those of ordinary skill in the art that botulinum toxins target one or more of three SNARE (Soluble NSF Attachment Protein Receptor) proteins VAMP (vesicle associated membrane protein, also known as synaptobrevin), SNAP-25 (synaptosomal-associated protein 25) and syntaxin (STX). BoNT/B cleaves the Q76 (P1 site)-F77 (P1′ site) (human numbering from here following) peptide bond of VAMP-2, BoNT/D and /DC cleave the K59-L60 bond, BoNT/F1 cleaves the Q58-K59 bond, and BoNT/G cleaves the A81-A82 bond. BoNT/F5 and BoNT/FA (also known as BoNT/H) hydrolyses the L54-E55 bond of VAMP-2, and BoNT/X cleaves R66-A67. BoNT/A cleaves the Q197-R198 bond at the C-terminus of SNAP-25, whereas BoNT/E hydrolyses the R180-I181 peptide bond. BoNT/C cleaves SNAP-25 (at R198-A199), STX-1A (at K253-A254), and STX-1B (at K252-A253). For example, the in vivo presence of BoNT/A may be determined by measuring the presence of the cleavage product of SNAP25 using an appropriate assay, such as an ELISA assay that utilizes one or more monoclonal antibodies. For example, the in vivo presence of OnabotulinumtoxinA may be determined using one or more mAbs such anti-SNAP-25 monoclonal antibody 4F3-2C1 (available from available from MyBioSource, Inc., San Diego, California, United States of America) and/or anti-SNAP-25 biotinylated antibody MBS423684 (available from MyBioSource, Inc., San Diego, California, United States of America).

In another aspect is provided any of the methods disclosed herein, wherein the second composition comprising one or more botulinum toxins is applied topically to the skin of the subject. In another aspect is provided any of the methods disclosed herein, wherein the second composition is applied to the skin of the subject in the form of a solution. In another aspect is provided any of the methods disclosed herein, wherein the second composition comprising one or more botulinum toxins is applied to the skin of the subject in the form of an aqueous solution. Solutions of botulinum toxins, including aqueous solutions, may be prepared by methods known to those of ordinary skill in the art. For example, a botulinum toxin product may be available in the form of a vacuum-dried or lyophilized solid packaged in a vial that contain appropriate excipients, such as human albumin, sodium chloride, sucrose, and sodium succinate. The vacuum-dried or lyophilized material may be prepared for use according to the methods disclosed herein by reconstitution with an appropriate diluent, such as preservative-free 0.9% sodium chloride injection USP, by slowly injecting the diluent into the vial and mixing the diluent and the vacuum-dried material by rotating the vial. Alternatively, a first aliquot of an appropriate diluent, such as preservative-free 0.9% sodium chloride injection USP, may be injected in the container containing the vacuum-dried materials and mixed to provide a first solution. A second aliquot of an appropriate diluent, such as preservative-free 0.9% sodium chloride injection USP, may be drawn into a syringe, followed by a given amount of the reconstituted toxin solution, followed by mixing the two aliquots in the syringe to provide a resulting solution having the desired concentration of the toxin. Generally, the date and time of reconstitution should be recorded and the reconstituted materials should generally be used within 24 hours after reconstitution. Any reconstituted materials should be stored under appropriate conditions, such as storage at a temperature of from about 2° C. to 8° C., following reconstitution and prior to use. Generally, reconstituted materials should be clear, colorless and free or particulate matter prior to use. Alternatively, the toxin product may be available as a pre-formed solution of a given concentration. The solution of toxin may be topically applied to the skin of a subject by drawing an appropriate amount of reconstituted solution from the container, such as a vial, by a syringe and then applying the reconstituted solution to the skin of the subject by an appropriate method, such as by means of a swab or a brush.

In another aspect is provided any of the methods disclosed herein, wherein the second composition comprises one or more botulinum toxin type selected from botulinum toxin type A, botulinum toxin type B, botulinum toxin type C₁, botulinum toxin type C₂, botulinum toxin type D, botulinum toxin type E, botulinum toxin type F and botulinum toxin type G. In another aspect is provided any of the methods disclosed herein, the one or more botulinum toxin type is selected from botulinum toxin type A and botulinum toxin type B. In another aspect is provided any of the methods disclosed herein, wherein the one or more botulinum toxin type is botulinum toxin type A. In another aspect is provided any of the methods disclosed herein, wherein the botulinum toxin type A is selected from onabotulinumtoxinA, abobotulinumtoxinA, and incobotulinumtoxinA. In another aspect is provided any of the methods disclosed herein, wherein the botulinum toxin type A is onabotulinumtoxinA. In another aspect is provided any of the methods disclosed herein, wherein the botulinum toxin type A is abobotulinumtoxinA. In another aspect is provided any of the methods disclosed herein, wherein the botulinum toxin type A is incobotulinumtoxinA. In another aspect is provided any of the methods disclosed herein, wherein the botulinum toxin type A is prabotulinumtoxinA. In another aspect is provided any of the methods disclosed herein, wherein the one or more botulinum toxin type is botulinum toxin type B. In another aspect is provided any of the methods disclosed herein, wherein the one or more botulinum toxin type B is rimabotulinumtoxinB. In another aspect is provided any of the methods disclosed herein, wherein the one or more botulinum toxin type is botulinum toxin type C₁. In another aspect is provided any of the methods disclosed herein, wherein the one or more botulinum toxin type is botulinum toxin type C₂. In another aspect is provided any of the methods disclosed herein, wherein the one or more botulinum toxin type is botulinum toxin type D. In another aspect is provided any of the methods disclosed herein, wherein the one or more botulinum toxin type is botulinum toxin type E. In another aspect is provided any of the methods disclosed herein, wherein the one or more botulinum toxin type E is EB-001A or EB-001T. In another aspect is provided any of the methods disclosed herein, wherein the one or more botulinum toxin type E is EB-001A. In another aspect is provided any of the methods disclosed herein, wherein the one or more botulinum toxin type E is EB-001T. In another aspect is provided any of the methods disclosed herein, wherein the one or more botulinum toxin type is botulinum toxin type F. In another aspect is provided any of the methods disclosed herein, wherein the one or more botulinum toxin type is botulinum toxin type G.

In another aspect is provided any of the methods disclosed herein, wherein the number of potency units of the second composition comprising one or more botulinum toxins applied to the skin of the subject is from about 1 to about 400 potency units. In another aspect is provided any of the methods disclosed herein, wherein the number of potency units of the second composition comprising one or more botulinum toxins applied to the skin of the subject is from about 10 units to about 400 units, or about 10 units to about 375 units, or from about 10 units to about 350 units, or from about 10 units to about 325 units, or from about 10 units to about 300 units, or from about 10 units to about 275 units, or from about 10 units to about 250 units, or from about 10 units to about 225 units, or from about 10 units to about 200 units, or from about 10 units to about 175 units, or from about 10 units to about 150 units, or from about 10 units to about 125 units, or from about 10 units to about 100 units, or from about 10 units to about 75 units, or from about 10 units to about 50 units, or from about 10 units to about 40 units, or from about 10 units to about 35 units, or from about 10 units to about 30 units, or from about 10 units to about 25 units, or from about 5 units to about 75 units, or from about 5 units to 50 units, or from about 5 units to about 45 units, or from about 5 units to about 40 units, or from about 5 units to about 35 units, or from about 5 units to about 30 units, or from about 5 units to about 25 units, or from about 5 units to about 20 units, or from about 5 units to about 15 units, or from about 5 units to about 10 units, or from about 15 units to about 100 units, or from about 20 units to about 100 units, or from about 25 units to about 100 units, or from about 35 units to about 100 units, or from about 40 units to about 100 units, or from about 50 units to about 100 units, or from about 60 units to about 100 units, or from about 70 units to about 100 units, or from about 80 units to about 100 units, or from about 90 units to about 100 units. In another aspect is provided any of the methods disclosed herein, wherein the number of potency units of the second composition comprising one or more botulinum toxins applied to the skin of the subject is about 1 unit, or about 2 units, or about 3 units, or about 4 units, or about 5 units, or about 6 units, or about 7 units, or about 8 units, or about 9 units, or about 10 units, or about 11 units, or about 12 units, or about 13 units, or about 14 units, or about 15 units, or about 20 units, or about 25 units, or about 30 units, or about 35 units, or about 40 units, or about 45 units, or about 50 units, or about 60 units, or about 70 units, or about 80 units, or about 90 units, or about 100 units. The number of potency units of the composition comprising at least one botulinum toxin that may be used according to the methods and kits disclosed herein may be determined by those of ordinary skill in the art. The potency of an individual botulinum toxin composition may be determined by methods known to those of ordinary skill in the art (see, for example, Fernandez-Salas et al., PLOS ONE, vol. 7, pp. e49516 (2012)), such as by an appropriate cell-based assay, use of a mouse LD₅₀ (mLD₅₀) bioassay, localized muscle paralysis (abdominal apoptosis) (see, for example, Sesardic et al., Pharmacol. Toxicol., vol. 78, pp. 283 to 288 (1996)), Digit Abduction Score assays (see, for example, Aoki et al., Toxicon., vol. 39, pp. 1815-1820 (2001)), rat or mouse phrenic nerve diaphragm (see, for example, Goschel et al., Exp Neurol., vol. 147, pp. 96-102 (1997)), rat intercostal muscle strip assays (see, for example, Huber et al., Altern Lab Anim., vol. 36, pp. 141-152 (2008)), and Rasetti-Escargucil et al., Toxicon., vol. 53, pp. 503-511 (2009)).

In another aspect is provided any of the methods disclosed herein, wherein the amount of the first composition comprising Spongilla applied to the skin of the subject is from about 0.5 grams to about 50 grams. In one aspect is provided any of the methods disclosed herein, wherein the amount of the first composition is measured as a dry weight. In another aspect is provided any of the methods disclosed herein, wherein the amount of the first composition comprising Spongilla applied to the skin of the subject is from about 0.5 grams to about 40 grams, or from about 0.5 grams to about 35 grams, or from about 0.5 grams to about 30 grams, or from about 0.5 grams to about 25 grams, or from about 0.5 grams to about 20 grams, or from about 0.5 grams to about 15 grams, or from about 0.5 grams to about 10 grams, or from about 0.75 grams to about 20 grams, or from about 0.75 grams to about 15 grams, or from about 0.75 grams to about 10 grams, or from about 1 gram to about 20 grams, or from about 1 gram to about 15 grams, or from about 1 gram to about 10 grams, or from about 1 gram to about 9 grams, or from about 1 gram to about 8 grams, or from about 1 gram to about 7 grams, or from about 1 gram to about 6 grams, or from about 1 gram to about 5 grams, or from about 1 gram to about 4 grams, or from about 1 gram to about 3 grams, or from about 1 gram to 2 grams. In another aspect is provided any of the methods disclosed herein wherein the amount of Spongilla applied to the skin, such as those disclosed above, are each measured as a dry weight.

In another aspect is provided any of the methods disclosed herein, wherein the amount of the first composition comprising Spongilla applied to the skin of the subject is about 0.5 grams, or about 0.75 grams, or about 1 gram, or about 1.25 grams, or about 1.5 grams, or about 1.75 grams, or about 2 grams, or about 2.25 grams, or about 2.5 grams, or about 2.75 grams, or about 3 grams, or about 3.25 grams, or about 3.5 grams, or about 3.75 grams, or about 4 grams, or about 4.25 grams, or about 4.5 grams, or about 4.75 grams, or about 5 grams, or about 5.25 grams, or about 5.5 grams, or about 5.75 grams, or about 6 grams, or about 6.25 grams, or about 6.5 grams, or about 7 grams, or about 7.25 grams, or about 7.5 grams, or about 7.75 grams, or about 8 grams, or about 8.25 grams, or about 8.5 grams, or about 8.75 grams, or about 9 grams, or about 9.25 grams, or about 9.5 grams, or about 9.75 grams, or about 10 grams, or about 11 grams, or about 12 grams, or about 13 grams, or about 14 grams, or about 15 grams, or about 16 grams, or about 17 grams, or about 18 grams, or about 19 grams, or about 20 grams, or about 25 grams, or about 35 grams, or about 40 grams, or about 45 grams, or about 50 grams, or about 75 grams, or about 100 grams, or about 250 grams, or about 500 grams, or about 750 grams, or about 1000 grams, in each case measured as a dry weight.

In another aspect is provided any of the methods disclosed herein, wherein the first composition is applied to the skin of the subject prior to the second composition being applied to the skin of the subject. In another aspect is provided any of the methods disclosed herein, wherein the first composition is applied to the skin of the subject and is permitted to dry on the skin of the subject prior to application of the second composition to the skin of the subject. In another aspect is provided any of the methods disclosed herein, wherein the first composition is applied to the skin of the subject in the form of an aqueous paste, wherein the aqueous component may be water or saline. In another aspect is provided any of the methods disclosed herein, wherein the aqueous paste further comprises hydrogen peroxide. In another aspect is provided any of the methods disclosed herein, wherein an aqueous solution of hydrogen peroxide is applied to the face of the subject following application of the first composition to the skin of the subject and prior to the application of the second composition to the skin of the subject. In another aspect is provided any of the methods disclosed herein, wherein the first composition further comprises hydrogen peroxide. In another aspect is provided any of the methods disclosed herein, wherein the hydrogen peroxide is an aqueous solution. In another aspect is provided any of the methods disclosed herein, wherein the aqueous solution comprises hydrogen peroxide at a w/w concentration of about 3%. In another aspect is provided any of the methods disclosed herein, wherein the second composition is permitted to dry on the skin of the subject following application to the skin of the subject.

In another aspect is provided any of the methods disclosed herein, wherein the second composition is applied to the skin of the subject prior to the first composition being applied to the skin of the subject. In another aspect is provided any of the methods disclosed herein, wherein the second composition is permitted to dry on the skin of the subject prior to the first composition being applied to the skin of the subject. In another aspect is provided any of the methods disclosed herein, wherein the first composition is applied to the skin of the subject in the form of an aqueous paste, wherein the aqueous portion may be derived from water or saline. In another aspect is provided any of the methods disclosed herein, wherein the aqueous paste further comprises hydrogen peroxide. In another aspect is provided any of the methods disclosed herein, wherein an aqueous solution of hydrogen peroxide is applied to the face of the subject following application of the first composition to the skin of the subject. In another aspect is provided any of the methods disclosed herein, wherein the hydrogen peroxide is an aqueous solution. In another aspect is provided any of the methods disclosed herein, wherein the aqueous solution comprises hydrogen peroxide at a w/w concentration of about 3%. In another aspect is provided any of the methods disclosed herein, wherein the first composition is permitted to dry on the skin of the subject.

In another aspect is provided any of the methods disclosed herein, wherein the first composition and the second composition are mixed together and the resulting mixture is applied to the skin of the subject. In another aspect is provided any of the methods disclosed herein, wherein the first composition is mixed with an aqueous solution of hydrogen peroxide prior to mixing with the second composition. In another aspect is provided any of the methods disclosed herein, wherein the mixture of the first composition and the second composition is further mixed with an aqueous solution of hydrogen peroxide prior to application to the skin of the subject. In another aspect is provided any of the methods disclosed herein, wherein the aqueous solution comprises hydrogen peroxide at a w/w concentration of about 3%. In another aspect is provided any of the methods disclosed herein, wherein the first composition comprising Spongilla and the second composition comprising one or more botulinum toxins is applied to the skin of the subject once per week.

In another aspect is provided any of the methods disclosed herein, wherein the subject applies the second composition comprising one or more botulinum toxins to the skin no more than once every 4 weeks.

In another aspect is provided any of the methods disclosed herein, wherein the first composition comprising Spongilla is applied to the skin of the subject at least once per week for at least one week. In another aspect is provided any of the methods disclosed herein, wherein the first composition comprising Spongilla is applied to the skin of the subject at least two times per week for at least one week, at least three times per week for at least one week, at least 4 times per week for at least one week, at least 5 times per week for at least one week, at least 6 times per week for at least one week, or at least 7 times per week for at least one week.

In another aspect is provided any of the methods disclosed herein, wherein the first composition comprising Spongilla is applied to the skin of the subject at least once per week for at least two weeks. In another aspect is provided any of the methods disclosed herein, wherein the first composition comprising Spongilla is applied to the skin of the subject at least at once per week for at least two weeks, at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In another aspect is provided any of the methods disclosed herein, wherein the first composition comprising Spongilla is applied to the skin of the subject once per week for 6 weeks.

In another aspect is provided any of the methods disclosed herein, wherein the first composition comprising Spongilla is applied to the skin of the subject once per week for 24 weeks, and the second composition comprising one or more botulinum toxins is applied to the skin of the subject only during the first week of treatment. In another aspect is provided any of the methods disclosed herein, wherein the first composition comprising Spongilla is applied to the skin of the subject once per week for 20 weeks, and the second composition comprising one or more botulinum toxins is applied to the skin of the subject only during the first week of treatment. In another aspect is provided any of the methods disclosed herein, wherein the first composition comprising Spongilla is applied to the skin of the subject once per week for 16 weeks, and the second composition comprising one or more botulinum toxins is applied to the skin of the subject only during the first week of treatment. In another aspect is provided any of the methods disclosed herein, wherein the first composition comprising Spongilla is applied to the skin of the subject once per week for 12 weeks, and the second composition comprising one or more botulinum toxins is applied to the skin of the subject only during the first week of treatment. In another aspect is provided any of the methods disclosed herein, wherein the first composition comprising Spongilla is applied to the skin of the subject once per week for 8 weeks, and the second composition comprising one or more botulinum toxins is applied to the skin of the subject only during the first week of treatment. In another aspect is provided any of the methods disclosed herein, wherein the first composition comprising Spongilla is applied to the skin of the subject once per week for 6 weeks, and the second composition comprising one or more botulinum toxins is applied to the skin of the subject only during the first week of treatment. In another aspect is provided any of the methods disclosed herein, wherein the first composition comprising Spongilla is applied to the skin of the subject once per week for 4 weeks, and the second composition comprising one or more botulinum toxins is applied to the skin of the subject only during the first week of treatment.

In another aspect is provided any of the methods disclosed herein, wherein the first composition comprising Spongilla and the second composition comprising one or more botulinum toxins is applied to the skin of the subject on at least one of the subject's face, back and chest. In another aspect is provided any of the methods disclosed herein, wherein the first composition comprising Spongilla and the second composition comprising one or more botulinum toxins is applied to the skin of the subject on the subject's face. In another aspect is provided any of the methods disclosed herein, wherein the first composition comprising Spongilla and the second composition comprising one or more botulinum toxins is applied to the skin of the subject on the subject's back. In another aspect is provided any of the methods disclosed herein, wherein the first composition comprising Spongilla and the second composition comprising one or more botulinum toxins is applied to the skin of the subject on the subject's chest.

In another aspect is provided any of the methods disclosed herein, wherein the skin of the subject is cleaned using a non-comedogenic cleanser, water, or a combination of a non-comedogenic cleanser and water following application of the first composition comprising Spongilla. In another aspect is provided any of the methods disclosed herein, wherein the skin of the subject is cleaned using a non-comedogenic cleanser, water, or a combination of a non-comedogenic cleanser and water following application of the second composition comprising one or more botulinum toxins to the skin of the subject. Non-comedogenic cleansers are those formulated not to cause blocked pores in the skin of subjects to which such cleansers are applied.

In another aspect is provided any of the methods disclosed herein, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides, atrohpic acne scars, and melasma. In another aspect is provided any of the methods disclosed herein, wherein the skin condition in the subject is selected from acne vulgaris, acne rosacea type 1, acne rosacea type 2, psoriasis, and hyperhidrosis. In another aspect is provided any of the methods disclosed herein, wherein the skin condition in the subject is acne vulgaris. In another aspect is provided any of the methods disclosed herein, wherein the skin condition in the subject is acne rosacea type 1. In another aspect is provided any of the methods disclosed herein, wherein the skin condition in the subject is acne rosacea type 2. In another aspect is provided any of the methods disclosed herein, wherein the skin condition in the subject is psoriasis. In another aspect is provided any of the methods disclosed herein, wherein the skin condition in the subject is hyperhidrosis.

Acne vulgaris is a common chronic skin disease involving blockage and/or inflammation of pilosebaceous units (hair follicles and their accompanying sebaceous gland). Acne can present as noninflammatory lesions, inflammatory lesions, or a mixture of both, affecting mostly the face but also the back and chest. The efficacy of a treatment regimen in a subject having acne vulgaris can be measured by methods known to those of ordinary skill in the art, such as by measurement of lesion counts and the investigator global assessment on the face in a subject such as found below:

| Score | Grade | Description |
|---|---|---|
| 0 | None | No evidence of facial acne vulgaris |
| 1 | Minimal | Few non-inflammatory lesions (comedones) are |
| | | present; a few inflammatory lesions (papules/pustules) |
| | | may be present; no nodulo-cystic lesions are allowed |
| 2 | Mild | Several to many non-inflammatory lesions |
| | | (comedones) are present; a few inflammatory lesions |
| | | (papules/pustules) are present; no nodulo-cystic |
| | | lesions are allowed |
| 3 | Moderate | Many non-inflammatory lesions (comedones) and |
| | | inflammatory lesions (papules/pustules) are present; no |
| | | nodulo-cystic lesions are allowed |
| 4 | Severe | Significant degree of inflammatory disease; |
| | | papules/pustules are a predominate feature; a few |
| | | nodulo-cystic lesions may be present; comedones may |
| | | be present |

Rosacea is well recognized as a chronic cutaneous disorder primarily of the convexities of the central face (cheeks, chin, nose, and central forehead), often characterized by remissions and exacerbations. Based on present knowledge, it is considered a syndrome, or typology, encompassing various combinations of such cutaneous signs as flushing, erythema., telangiectasia., edema, papules, pustules, ocular lesions, and rhinophyma. In most cases, some rather than all of these stigmata appear in any given subject. Acne rosacea type-1, or erythematotelangiectatic rosacea, is mainly characterized by flushing and persistent central facial erythema. The appearance of telangiectases is common but not essential for a diagnosis of this subtype. Central facial edema, stinging and burning sensations, and roughness or scaling may also be reported. A history of flushing alone is common among subjects presenting with erythematotelangiectatic rosacea. The efficacy of a treatment regimen in a subject having acne rosacea type-ilcan be measured by methods known to those of ordinary skill in the art, such as by use of the Clinician Erythema Assessment (CEA), a 5-point grading scale of facial erythema severity, and Subject Self-Assessment (SSA) shown below:

| Grade | Category | Description |
|---|---|---|
| 0 | None | Clear skin with no signs of erythema |
| 1 | Minimal | Almost clear of erythema, slight redness |
| 2 | Mild | Mild erythema, definite redness |
| 3 | Moderate | Moderate erythema, marked redness |
| 4 | Severe | Severe erythema, fiery redness |

| Grade | Category | Description |
|---|---|---|
| 0 | None | Clear of redness |
| 1 | Minimal | Almost clear of redness |
| 2 | Mild | Somewhat more redness than preferred |
| 3 | Moderate | More redness than preferred |
| 4 | Severe | Completely unacceptable redness |

Acne rosacea type 2 (papulopustular) is characterized by persistent central facial erythema with transient papules or pustules or both in a central facial distribution. However, papules and pustules also may occur periorificially (that is, they may occur in the perioral, perinasal, or periocular areas). The papulopustular subtype resembles acne vulgaris, except that comedones are absent. Rosacea and acne may occur concomitantly, and such subjects may have comedones as well as the papules and pustules of rosacea. Burning and stinging sensations may be reported by subjects with papulopustular rosacea. This subtype has often been seen after or in combination with subtype 1, including the presence of telangiectases. The telangiectases may be obscured by persistent erythema., papules, or pustules, and tend to become more visible after successful treatment of these masking components. The efficacy of a treatment regimen in a subject having acne rosacea type-2 can be measured by methods known to those of ordinary skill in the art, such as by total lesion counts in the area of the skin of the subject undergoing treatment and an investigator global assessment as shown below:

| Grade | Category | Description |
|---|---|---|
| 0 | Clear | No papules and/or pustules |
| 1 | Almost Clear | Rare papules and/or pustules |
| 2 | Mild | Few papules and/or pustules |
| 3 | Moderate | Pronounced number of papules and/or pustules |
| | | (but less than numerous papules and/or pustules) |
| 4 | Severe | Numerous papules and/or pustules, occasionally |
| | | with confluent areas of inflamed lesions |

Psoriasis is a skin condition that speeds up the life cycle of skin cells. It causes cells to build up rapidly on the surface of the skin. The extra skin cells form scales and red patches that are itchy and sometimes painful. The symptoms a subject having psoriasis may present include red patches of skin covered with thick, silvery scales, small scaling spots (commonly seen in children), dry, cracked skin that may bleed, itching, burning or soreness, thickened, pitted or ridged nails, and swollen and stiff joints. The efficacy of a treatment regimen in a subject having acne psoriasis can be measured by methods known to those of ordinary skill in the art, such as by use of the Psoriasis Area and Severity Index (PASI). Use of PASI involves dividing the body of the subject into four sections (head (H) (10% of a person's skin); arms (A) (20%); trunk (T) (30%); legs (L) (40%)). Each of these areas is scored by itself, and then the four scores are combined into the final PASI. For each section, the percent of area of skin involved, is estimated and then transformed into a grade from 0 to 6 as in the table below. Within each area, the severity is estimated by three clinical signs: erythema (redness), induration (thickness) and desquamation (scaling). Severity parameters are measured on a scale of 0 to 4, from none to maximum. The sum of all three severity parameters is then calculated for each section of skin, multiplied by the area score for that area and multiplied by weight of respective section (0.1 for head, 0.2 for arms, 0.3 for body and 0.4 for legs).

| Score | Description |
|---|---|
| 0 | 0% of involved area |
| 1 | <10% of involved area |
| 2 | 10-29% of involved area |
| 3 | 30-49% of involved area |
| 4 | 50-69% of involved area |
| 5 | 70-89% of involved area |
| 6 | 90-100% of involved area |

Hyperhidrosis is a condition characterized by abnormally increased sweating, generally in excess of that required for regulation of body temperature. Although primarily a physical burden, hyperhidrosis can deteriorate quality of life from a psychological, emotional, and social perspective. The efficacy of a treatment regimen in a subject having hyperhidrosis can be measured by methods known to those of ordinary skill in the art, such as by use of the hyperhidrosis disease severity scale (HDSS), which is a 4-point scale designed to assess the severity of primary axillary hyperhidrosis in everyday clinical practice or in clinical research. The HDSS can be administered by an interviewer or self-completed by the subject. The HDSS assesses subject severity based on the extent of excessive sweating-related impairment of daily activities. Subjects rate the severity as: 1=my underarm sweating is never noticeable and never interferes with my daily activities; 2=my underarm sweating is tolerable but sometimes interferes with my daily activities; 3=my underarm sweating is barely tolerable and frequently interferes with my daily activities; or 4=my underarm sweating is intolerable and always interferes with my daily activities.

The efficacy of a treatment regimen in a subject having hyperhidrosis may also be measured by use of the Axillary Sweating Daily Diary (ASDD), Item 2, and/or the Axillary Sweating Daily Diary-Children (ASDD-C), which are validated patient-reported outcome measure to assess axillary hyperhidrosis sweating severity. The ADSS Item 2 asks subjects to rate their underarm sweating during the last 24-hour period at its worst using a 10-point scale, in which a score of zero (0) represents “no sweating at all,” and a score of ten (10) represents “worst possible sweating.” See Glaser et. al., J. Am. Acad. Dermatol., 2018, Supplemental FIG. 1 for a description of the ASDD, including Item 2, and the ASDD-C.

Alopecia areata is an autoimmune skin disease, causing hair loss on the scalp, face and sometimes on other areas of the body. In fact, it affects as many as 6.8 million people in the U.S. The efficacy of a treatment regimen in a subject having alopecia areata can be measured by methods known to those of ordinary skill in the art, such as by use fixed hair counts, and loose hair counts on a subject's pillow.

Androgenic alopecia is a genetically determined disorder characterized by the gradual conversion of terminal hairs into indeterminate, and finally into vellus, hairs. It is an extremely common disease that affects men and women. Subjects suffering from androgenic alopecia generally display symptoms such as a gradual onset of hair loss, increased hair shedding, transition in the involved areas from large, thick, pigmented terminal hairs to thinner, shorter, indeterminate hairs and finally to short, wispy, nonpigmented vellus hairs, the end result of which may be an area of total denudation; this area varies from subject to subject and is usually most marked at the vertex. The efficacy of a treatment regimen in a subject having androgenic alopecia can be measured by methods known to those of ordinary skill in the art, such as by use fixed hair counts, and loose hair counts on a subject's pillow.

Keloids are raised, reddish nodules that develop at the site of an injury. After a wound has occurred to the skin both skin cells and connective tissue cells (fibroblasts) begin multiplying to repair the damage. A scar is made up of ‘connective tissue’, gristle-like fibers deposited in the skin by the fibroblasts to hold the wound closed. With keloids, the fibroblasts continue to multiply even after the wound is filled in. Thus, keloids project above the surface of the skin and form large mounds of scar tissue. Keloids may form on any part of the body, although the upper chest, shoulders and upper back are especially prone to keloid formation. Symptoms include pigmentation of the skin, itchiness, redness, unusual sensations and pain. Darkly pigmented people seem to be more prone to forming keloids. Men and women are equally affected. Keloids are considered a benign tumor, but they are mainly a cosmetic nuisance and never become malignant. Operating on a keloid usually stimulates more scar tissue to form; so many subjects having keloids may be told that there are no available treatments. Hypertrophic scars appear like, and are more common than, keloids, although they do not generally grow as large as keloids, may fade with time, and occur in all racial groups. The efficacy of a treatment regimen in a subject having keloids and/or hypertrophic scars can be measured by methods known to those of ordinary skill in the art, such as by the use the Vancouver Scar Scale (VSS), Manchester Scar Scale (MSS), Subject and Observer Scar Assessment Scale (POSAS), Visual Analog Scale (VAS), and Stony Brook Scar Evaluation Scale (SBSES).

Hidradenitis suppurativa is a disease that usually begins as pimple-like bumps on the skin, which tend to develop in places that everyday pimples do not appear and is most common on the underarms and groin. If hidradenitis suppurativa worsens, the pimple-like bumps can grow deep into the skin and become painful and can rupture. As the deep bumps heal, scars can form, and some subjects develop tunnel-like tracts under their skin, forming scars, which can thicken. When thick scars form in the underarm, moving the arm can be difficult. Thick scars in the groin area can make walking difficult. The efficacy of a treatment regimen in a subject suffering from hidradenitis suppurativa can be measured by methods known to those of ordinary skill in the art, such as by the visual count of lesion counts in the affected areas of a subject's skin.

Raynaud's phenomenon is a type of vascular disease characterized by a pale to blue to red sequence of color changes of the digits, most commonly after exposure to cold. The cause of Raynaud's phenomenon is unknown, although abnormal nerve control of blood-vessel diameter and nerve sensitivity to cold are suspected of being involved. Symptoms of Raynaud's phenomenon depend on the severity, frequency, and duration of the blood-vessel spasm. The efficacy of a treatment regimen in a subject suffering from Raynaud's phenomenon can be measured by methods known to those of ordinary skill in the art, such as measurements of digital pulp temperature, photographic assessment of the affected areas, and a visual analogue scale for pain in the affected areas.

Post-herpetic neuralgia is generally considered a complication of shingles, which is caused by the chickenpox (herpes zoster) virus. Postherpetic neuralgia affects nerve fibers and skin, causing burning pain that lasts long after the rash and blisters of shingles disappear. The signs and symptoms of postherpetic neuralgia are generally limited to the area in a subject's skin where the shingles outbreak first occurred. Signs and symptoms of post-herpetic neuralgia may include pain that lasts 3 months or longer after the shingles rash has healed sensitivity to light touch, and itching and numbness in the affected area. The efficacy of a treatment regimen in a subject suffering from post-herpetic neuralgia can be measured by methods known to those of ordinary skill in the art, use of a visual analogue scale for pain in the affected areas.

Hailey-Hailey Disease (familial benign pemphigus) is a genetic disorder that causes blisters to form on the skin and is characterized by outbreaks of rashes and blisters in the skin, usually in the folds of the skins, but also often over large areas of the body. The painful blisters break and sometimes become infected and raw, with new blisters forming over raw skin in a sometimes seemingly unending cycle of outbreaks. The cause of the disease is a haploinsufficiency of the enzyme ATP2C1, which encodes the protein hSPCA1. A mutation on one copy of the gene causes only half of this necessary protein to be made and the cells of the skin do not adhere together properly due to malformation of intercellular desmosomes, causing acantholysis, blisters and rashes. The efficacy of a treatment regimen in a subject suffering from Hailey-Hailey Disease can be measured by methods known to those of ordinary skill in the art, such as counting the total lesion counts in a subject, wherein a reduction in the total lesion count indicates the treatment regimen is having a positive effect.

Linear IgA bullous dermatosis (LABD) is a rare subepidermal blistering disease due to an autoimmune reaction against basement membrane proteins such as the lamina lucida and sublamina densa. The basement membrane anchors the epidermis to the dermis and helps to stabilize the skin. When IgA antibodies target such proteins, the basement membrane destabilizes resulting in tense blister formation. In the majority of LABD cases, the cause is unknown or idiopathic. Furthermore, more than half of all childhood cases tend to remit over a mean course of two to four years. Adults may have a more protracted course and LABD has been shown to occur in those with internal malignancy, infection, and other autoimmune diseases like rheumatoid arthritis or dermatomyositis. Other cases of LABD are drug-induced often due to vancomycin and subjects can break out as early after the first dose of vancomycin in some cases. The efficacy of a treatment regimen in a subject suffering from LABD can be measured by methods known to those of ordinary skill in the art, such as counting the total lesion counts in a subject, wherein a reduction in the total lesion count indicates the treatment regimen is having a positive effect.

Epidermolysis bullosa simplex (EBS) is a chronic vesicular disorder with characteristic manifestations, from birth to infancy, of intraepidermal vesicle and milia formation on the hand, elbow, or knee due to minimal trauma. It is a genetic disorder that is caused by a dominant-negative mutation in either the keratin 5 (KRT5) or the keratin 14 (KRT14) gene. EBS is sub-categorized by its clinical manifestation into the systemic (Koebner), localized (Weber-Cockayne), and herpetiform (Dowling-Meara) 1 types. The localized type of EBS is the mildest form of the subtypes that involves easy development of vesicles on the palms and soles from minimal mechanical trauma. According to molecular genetic studies of EBS, there are mutations in KRT5 and KRT14, which contribute to skeletons on hemidesmosome in keratinocytes located in the basal layer near the dermoepidermal junction. Mutations in each subtype of EBS vary in location and severity2,3. The efficacy of a treatment regimen in a subject suffering from EBS can be measured by methods known to those of ordinary skill in the art, such as counting the total lesion counts in a subject, wherein a reduction in the total lesion count indicates the treatment regimen is having a positive effect.

Darier Disease is a skin condition characterized by wart-like blemishes on the body. The blemishes are usually yellowish in color, hard to the touch, mildly greasy, and can emit a strong odor. The most common sites for blemishes are the scalp, forehead, upper arms, chest, back, knees, elbows, and behind the ear. The mucous membranes can also be affected, with blemishes on the roof of the mouth (palate), tongue, inside of the cheek, gums, and throat. Other features of Darier disease include nail abnormalities, such as red and white streaks in the nails with an irregular texture, and small pits in the palms of the hands and soles of the feet. The wart-like blemishes characteristic of Darier disease usually appear in late childhood to early adulthood. The severity of the disease varies over time; affected people experience flare-ups alternating with periods when they have fewer blemishes. The appearance of the blemishes is influenced by environmental factors. Most people with Darier disease will develop more blemishes during the summertime when they are exposed to heat and humidity. UV light; minor injury or friction, such as rubbing or scratching; and ingestion of certain medications can also cause an increase in blemishes. The efficacy of a treatment regimen in a subject suffering from Darier Disease can be measured by methods known to those of ordinary skill in the art, such as counting the total lesion counts and measuring the size of the lesions in a subject, wherein a reduction in the total lesion count indicates the treatment regimen is having a positive effect.

Pachyonchia Congenita is a condition that primarily affects the nails and skin. The signs and symptoms of this condition in a subject usually become apparent within the first few months of a subject's life. Almost everyone with pachyonychia congenita has hypertrophic nail dystrophy, which causes the fingernails and toenails to become thick and abnormally shaped. Many affected children also develop very painful blisters and calluses on the soles of the feet and, less commonly, on the palms of the hands. This condition is known as palmoplantar keratoderma. Severe blisters and calluses on the feet can make it painful or impossible to walk. Pachyonychia congenita can have several additional features, which vary among affected individuals. These features include thick, white patches on the tongue and inside of the cheeks (oral leukokeratosis); bumps called follicular keratoses that develop around hair follicles on the elbows, knees, and waistline; cysts in the armpits, groin, back, or scalp; and excessive sweating on the palms and soles (palmoplantar hyperhidrosis). Some affected individuals also develop widespread cysts called steatocystomas, which are filled with an oily substance called sebum that normally lubricates the skin and hair. Some babies with pachyonychia congenita have prenatal or natal teeth, which are teeth that are present at birth or in early infancy. Rarely, pachyonychia congenita can affect the voice box (larynx), potentially leading to hoarseness or breathing problems. The efficacy of a treatment regimen in a subject suffering from pachyonchia congenita can be measured by methods known to those of ordinary skill in the art, such as counting the total number of blisters and measuring the size of the blisters in the affected area on a subject.

Aquagenic keratoderma (AK) is a skin disorder also known as acquired aquagenic palmoplantar keratoderma, transient reactive papulotranslucent acrokeratoderma, aquagenic wrinkling of the palms or aquagenic syringeal acrokeratoderma. The main characteristic of the disorder is skin wrinkling with edema of palms/soles, whitish papules, pruritus, burning, and pain after contact with water. Prolongation of water exposure and temperature of the water affect the rate and intensity of lesion development; however, the pathogenesis of AK is poorly understood. The efficacy of a treatment regimen in a subject suffering from AK can be measured by methods known to those of ordinary skill in the art, such as counting the total number of lesions in a subject, the visual analogue pain score, and the visual analogue pruritis score.

Notalgia paresthetic is a sensory neuropathic syndrome of the midback skin, classically described as the unilateral infrascapular area. It is primarily a localized pruritus and dysesthesia syndrome, and it may present with episodic itching or pain on a small patch of the mid back, usually an area of skin just past easy reach. The correlation of notalgia paraesthetica localization with corresponding degenerative changes in the spine suggest that spinal nerve impingement may be a contributing cause, but subjects may have other conditions that predispose them to peripheral neuropathies, such as nerve damage. The efficacy of a treatment regimen in a subject suffering from notalgia paraesthetica can be measured by methods known to those of ordinary skill in the art, such as counting the total number of lesions in a subject, the visual analogue pain score, and the visual analogue pruritis score.

Pompholyx (dyshidrotic eczema) is a skin condition in which very small, fluid-filled blisters appear on the palms of a subject's hands, sides of the fingers, and soles of the feet. The blisters that occur in dyshidrosis may cause intense itching and, once dried, may cause a subject's skin to appear scaly. The blisters typically recur, sometimes before a subject's skin heals completely from the previous blisters. The efficacy of a treatment regimen in a subject suffering from dyshidrotic eczema can be measured by methods known to those of ordinary skill in the art, such as observing the signs and symptoms of eczema, the visual analogue pain score, and the visual analogue pruritis score.

Chromhidrosis is a condition characterized by the secretion of colored sweat and is caused by the deposition of lipofuscin in the sweat glands. It normally affects the apocrine glands, mainly on the face and underarms. The efficacy of a treatment regimen in a subject suffering from chromhidrosis can be measured by methods known to those of ordinary skill in the art, such as observing the signs of sweat and the odor of sweat in an affected subject.

Bromhidrosis, also known as osmidrosis, is a condition of abnormal or offensive body odor, largely determined by apocrine gland secretion, although other sources may play a role. Sudoriferous (sweat) glands are divided into two types: apocrine and eccrine and there is some crossover in some subjects. The efficacy of a treatment regimen in a subject suffering from bromhidrosis can be measured by methods known to those of ordinary skill in the art, such as observing the odor of sweat in an affected subject.

Eccrine nevus is a disease, which may be present at birth or at an early age. It is more often associated with localized hyperhidrosis, while cases not associated have also been reported. It is usually characterized histologically by the increase in number or size of structurally normal eccrine glands. The efficacy of a treatment regimen in a subject suffering from eccrine nevus can be measured by methods known to those of ordinary skill in the art, use of the Hyperhidrosis Disease Severity Scale (HDSS), and measuring the number sweat episodes per month in an affected subject.

Facial rhytides is a condition in subjects that is associated with moderate to severe glabellar lines associated with corrugator and/or procerus muscle activity, moderate to severe lateral canthal lines associated with orbicularis oculi activity, and/or moderate to severe forehead lines associated with frontalis muscle activity. The efficacy of a treatment regimen in a subject having facial rhytides can be measured by methods known to those of ordinary skill in the art, including a 4-point Facial Wrinkle Scale (FWS; 0=none, 1=mild, 2=moderate, 3=severe).

Atrophic acne scarring can occur in subjects suffering from acne. The efficacy of a treatment regimen in a subject suffering from atrophic acne scarring can be measured by methods known to those of ordinary skill in the art, including the Self-assessment of Clinical Acne-Related Scars (SCARS) and the Facial Acne Scar Quality of Life (FASQoL) tools.

In another aspect is provided a method of treating a skin condition in a subject, comprising applying to the skin of the subject a first composition comprising Spongilla, and a second composition comprising one or more botulinum toxins, wherein (a) the second composition is comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G; and (b) the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another embodiment are methods, wherein the one or more botulinum toxin type is selected from botulinum toxin type A and botulinum toxin type B. In another embodiment are methods, wherein the one or more botulinum toxin type is botulinum toxin type A. In another embodiment are methods, wherein the botulinum toxin type A is selected from onabotulinumtoxinA, abobotulinumtoxinA, incobotulinumtoxinA, prabotulinumtoxinA, and daxibotulinumtoxinA. In another embodiment are methods, wherein the botulinum toxin type A is onabotulinumtoxinA. In another embodiment are methods, wherein the botulinum toxin type A is abobotulinumtoxinA. In another embodiment are methods, wherein the botulinum toxin type A is incobotulinumtoxinA. In another aspect is provided any of the methods disclosed herein, wherein the botulinum toxin type A is prabotulinumtoxinA. In another aspect is provided any of the methods disclosed herein, wherein the botulinum toxin type A is daxibotulinumtoxinA. In another embodiment are methods, wherein the one or more botulinum toxin type is botulinum toxin type B. In another aspect is provided any of the methods disclosed herein, wherein the one or more botulinum toxin type B is rimabotulinumtoxinB. In another embodiment are methods, wherein the skin condition in the subject is selected from acne vulgaris, acne rosacea type 1, acne rosacea type 2, psoriasis, and hyperhidrosis. In another aspect is provided any of the methods disclosed herein, wherein the one or more botulinum toxin type is botulinum toxin type E. In another aspect is provided any of the methods disclosed herein, wherein the one or more botulinum toxin type E is EB-001A or EB-001T. In another aspect is provided any of the methods disclosed herein, wherein the one or more botulinum toxin type E is EB-001A. In another aspect is provided any of the methods disclosed herein, wherein the one or more botulinum toxin type E is EB-001T. In another embodiment are methods, wherein the skin condition in the subject is selected from acne vulgaris, acne rosacea type 1, acne rosacea type 2, psoriasis, and hyperhidrosis. In another embodiment are methods, wherein the skin condition in the subject is acne vulgaris. In another embodiment are methods, wherein the skin condition in the subject is acne rosacea type 1. In another embodiment are methods, wherein the skin condition in the subject is acne rosacea type 2. In another embodiment are methods, wherein the skin condition in the subject is psoriasis. In another embodiment are methods, wherein the skin condition in the subject is hyperhidrosis. In another embodiment are methods, wherein the Spongilla is Spongilla lacustris. In another embodiment are methods, wherein the second composition comprising one or more botulinum toxins is applied topically to the skin of the subject. In another embodiment are methods, wherein the second composition comprising one or more botulinum toxins is in the form of an aqueous solution. In another embodiment are methods, wherein the second composition is applied to the skin of the subject in the form of a solution. In another embodiment are methods, wherein the second composition is in the form of an aqueous solution. In another embodiment are methods, wherein the amount of the first composition comprising Spongilla applied to the skin of the subject is from about 0.5 grams to about 50 grams, measured as a dry weight. In another embodiment are methods, wherein the first composition is applied to the skin of the subject in the form of a paste. In another embodiment are methods, wherein the paste further comprises water or saline. In another embodiment are methods, wherein the paste is prepared by mixing a powder comprising Spongilla and an aqueous solution comprising hydrogen peroxide. In another embodiment are methods, wherein the aqueous solution comprises hydrogen peroxide at a w/w concentration of about 3%. In another embodiment are methods, wherein the first composition is applied to the skin of the subject prior to the second composition being applied to the skin of the subject. In another embodiment are methods, wherein the first composition is applied to the skin of the subject and is permitted to dry on the skin of the subject prior to application of the second composition to the skin of the subject. In another embodiment are methods, wherein the first composition is applied to the skin of the subject in the form of an aqueous paste, wherein the aqueous portion is derived from water or saline. In another embodiment are methods, wherein the aqueous paste further comprises hydrogen peroxide. In another embodiment are methods, wherein an aqueous solution of hydrogen peroxide is applied to the face of the subject following application of the first composition to the skin of the subject and prior to the application of the second composition to the skin of the subject. In another embodiment are methods, wherein the first composition further comprises hydrogen peroxide. In another embodiment are methods, wherein the hydrogen peroxide is an aqueous solution. In another embodiment are methods, wherein the aqueous solution comprises hydrogen peroxide at a w/w concentration of about 3%. In another embodiment are methods, wherein the second composition is permitted to dry on the skin of the subject following application to the skin of the subject. In another embodiment are methods, wherein the second composition is applied to the skin of the subject prior to the first composition being applied to the skin of the subject. In another embodiment are methods, wherein the second composition is permitted to dry on the skin of the subject prior to the first composition being applied to the skin of the subject. In another embodiment are methods, wherein the first composition is applied to the skin of the subject in the form of an aqueous paste, wherein the aqueous portion is derived from water or saline. In another embodiment are methods, wherein the aqueous paste further comprises hydrogen peroxide. In another embodiment are methods, wherein an aqueous solution of hydrogen peroxide is applied to the face of the subject following application of the first composition to the skin of the subject. In another embodiment are methods, wherein the hydrogen peroxide is an aqueous solution. In another embodiment are methods, wherein the aqueous solution comprises hydrogen peroxide at a w/w concentration of about 3%. In another embodiment are methods, wherein the first composition is permitted to dry on the skin of the subject. In another embodiment are methods, wherein the first composition and the second composition are mixed together, and the resulting mixture is applied to the skin of the subject. In another embodiment are methods, wherein the first composition is mixed with an aqueous solution of hydrogen peroxide prior to mixing with the second composition. In another embodiment are methods, wherein the mixture of the first composition and the second composition is further mixed with an aqueous solution of hydrogen peroxide prior to application to the skin of the subject. In another embodiment are methods, wherein the aqueous solution comprises hydrogen peroxide at a w/w concentration of about 3%. In another embodiment are methods, wherein the first composition and the second composition are applied to the skin of the subject once per week. In another embodiment are methods, wherein the second composition is applied to the skin of the subject no more than once every 4 weeks. In another embodiment are methods, wherein the first composition is applied to the skin of the subject at least once per week for at least one week. In another embodiment are methods, wherein the first composition is applied to the skin of the subject once per week for 6 weeks. In another embodiment are methods, wherein the first composition and the second composition are applied to the skin of the subject on at least one of the subject's face, back and chest. In another embodiment are methods, wherein the first composition and the second composition are applied to the skin of the subject on the subject's face. In another embodiment are methods, wherein the first composition and the second composition are applied to the skin of the subject on the subject's back. In another embodiment are methods, wherein the first composition c and the second composition comprising are applied to the skin of the subject on the subject's chest.

In one aspect is provided a method of treating a skin condition in a subject, comprising applying to the skin of the subject a first composition and a second composition, wherein (a) the first composition comprises Spongilla powder; (b) the second composition is comprises one or more botulinum toxin type selected from botulinum toxin type A, and botulinum toxin type B; and (c) the skin condition in the subject is selected from acne vulgaris, acne rosacea type 1, acne rosacea type 2, psoriasis, hyperhidrosis, alopecia areata, androgenic alopecia, keloids and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another embodiment are methods, wherein the one or more botulinum toxin type is botulinum toxin type A. In another embodiment are methods, wherein the botulinum toxin type A is selected from onabotulinumtoxinA, abobotulinumtoxinA, incobotulinumtoxinA, prabotulinumtoxinA, and daxibotulinumtoxinA. In another embodiment are methods, wherein the botulinum toxin type A is onabotulinumtoxinA. In another embodiment are methods, wherein the botulinum toxin type A is abobotulinumtoxinA. In another embodiment are methods, wherein the botulinum toxin type A is incobotulinumtoxinA. In another aspect is provided any of the methods disclosed herein, wherein the botulinum toxin type A is prabotulinumtoxinA. In another aspect is provided any of the methods disclosed herein, wherein the botulinum toxin type A is daxibotulinumtoxinA. In another embodiment are methods, wherein the one or more botulinum toxin type is botulinum toxin type B. In another aspect is provided any of the methods disclosed herein, wherein the one or more botulinum toxin type B is rimabotulinumtoxinB. In another embodiment are methods, wherein the one or more botulinum toxin type is botulinum toxin type E. In another aspect is provided any of the methods disclosed herein, wherein the one or more botulinum toxin type E is EB-001A or EB-001T. In another aspect is provided any of the methods disclosed herein, wherein the one or more botulinum toxin type E is EB-001A. In another aspect is provided any of the methods disclosed herein, wherein the one or more botulinum toxin type E is EB-001T. In another embodiment are methods, wherein the skin condition in the subject is selected from acne vulgaris, acne rosacea type 1, acne rosacea type 2, psoriasis, and hyperhidrosis. In another embodiment are methods, wherein the skin condition in the subject is acne vulgaris. In another embodiment are methods, wherein the skin condition in the subject is acne rosacea type 1. In another embodiment are methods, wherein the skin condition in the subject is acne rosacea type 2. In another embodiment are methods, wherein the skin condition in the subject is psoriasis. In another embodiment are methods, wherein the skin condition in the subject is hyperhidrosis. In another embodiment are methods, wherein the Spongilla is Spongilla lacustris. In another embodiment are methods, wherein the second composition comprising one or more botulinum toxins is applied topically to the skin of the subject. In another embodiment are methods, wherein the second composition comprising one or more botulinum toxins is in the form of an aqueous solution. In another embodiment are methods, wherein the second composition is applied to the skin of the subject in the form of a solution. In another embodiment are methods, wherein the second composition is in the form of an aqueous solution. In another embodiment are methods, wherein the amount of the first composition comprising Spongilla applied to the skin of the subject is from about 0.5 grams to about 50 grams, measured as a dry weight. In another embodiment are methods, wherein the first composition is applied to the skin of the subject in the form of a paste. In another embodiment are methods, wherein the paste further comprises water or saline. In another embodiment are methods, wherein the paste is prepared by mixing a powder comprising Spongilla and an aqueous solution comprising hydrogen peroxide. In another embodiment are methods, wherein the aqueous solution comprises hydrogen peroxide at a w/w concentration of about 3%. In another embodiment are methods, wherein the first composition is applied to the skin of the subject prior to the second composition being applied to the skin of the subject. In another embodiment are methods, wherein the first composition is applied to the skin of the subject and is permitted to dry on the skin of the subject prior to application of the second composition to the skin of the subject. In another embodiment are methods, wherein the first composition is applied to the skin of the subject in the form of an aqueous paste, wherein the aqueous portion is derived from water or saline. In another embodiment are methods, wherein the aqueous paste further comprises hydrogen peroxide. In another embodiment are methods, wherein an aqueous solution of hydrogen peroxide is applied to the face of the subject following application of the first composition to the skin of the subject and prior to the application of the second composition to the skin of the subject. In another embodiment are methods, wherein the first composition further comprises hydrogen peroxide. In another embodiment are methods, wherein the hydrogen peroxide is an aqueous solution. In another embodiment are methods, wherein the aqueous solution comprises hydrogen peroxide at a w/w concentration of about 3%. In another embodiment are methods, wherein the second composition is permitted to dry on the skin of the subject following application to the skin of the subject. In another embodiment are methods, wherein the second composition is applied to the skin of the subject prior to the first composition being applied to the skin of the subject. In another embodiment are methods, wherein the second composition is permitted to dry on the skin of the subject prior to the first composition being applied to the skin of the subject. In another embodiment are methods, wherein the first composition is applied to the skin of the subject in the form of an aqueous paste, wherein the aqueous portion is derived from water or saline. In another embodiment are methods, wherein the aqueous paste further comprises hydrogen peroxide. In another embodiment are methods, wherein an aqueous solution of hydrogen peroxide is applied to the face of the subject following application of the first composition to the skin of the subject. In another embodiment are methods, wherein the hydrogen peroxide is an aqueous solution. In another embodiment are methods, wherein the aqueous solution comprises hydrogen peroxide at a w/w concentration of about 3%. In another embodiment are methods, wherein the first composition is permitted to dry on the skin of the subject. In another embodiment are methods, wherein the first composition and the second composition are mixed together, and the resulting mixture is applied to the skin of the subject. In another embodiment are methods, wherein the first composition is mixed with an aqueous solution of hydrogen peroxide prior to mixing with the second composition. In another embodiment are methods, wherein the mixture of the first composition and the second composition is further mixed with an aqueous solution of hydrogen peroxide prior to application to the skin of the subject. In another embodiment are methods, wherein the aqueous solution comprises hydrogen peroxide at a w/w concentration of about 3%. In another embodiment are methods, wherein the first composition and the second composition are applied to the skin of the subject once per week. In another embodiment are methods, wherein the second composition is applied to the skin of the subject no more than once every 4 weeks. In another embodiment are methods, wherein the first composition is applied to the skin of the subject at least once per week for at least one week. In another embodiment are methods, wherein the first composition is applied to the skin of the subject once per week for 6 weeks. In another embodiment are methods, wherein the first composition and the second composition are applied to the skin of the subject on at least one of the subject's face, back and chest. In another embodiment are methods, wherein the first composition and the second composition are applied to the skin of the subject on the subject's face. In another embodiment are methods, wherein the first composition and the second composition are applied to the skin of the subject on the subject's back. In another embodiment are methods, wherein the first composition c and the second composition comprising are applied to the skin of the subject on the subject's chest.

In one aspect is provided a method of treating a skin condition in a subject, comprising applying to the skin of the subject a first composition and a second composition, wherein (a) the first composition comprises Spongilla lacustris powder; (b) the second composition comprises one or more botulinum toxin type selected from botulinum toxin type A; and (c) the skin condition in the subject is selected from acne vulgaris, acne rosacea type 1, acne rosacea type 2, psoriasis, and hyperhidrosis. In another embodiment are methods, wherein the botulinum toxin type A is selected from onabotulinumtoxinA, abobotulinumtoxinA, incobotulinumtoxinA, prabotulinumtoxinA, and daxibotulinumtoxinA. In another embodiment are methods, wherein the botulinum toxin type A is onabotulinumtoxinA. In another embodiment are methods, wherein the botulinum toxin type A is abobotulinumtoxinA. In another embodiment are methods, wherein the botulinum toxin type A is incobotulinumtoxinA. In another aspect is provided any of the methods disclosed herein, wherein the botulinum toxin type A is prabotulinumtoxinA. In another aspect is provided any of the methods disclosed herein, wherein the botulinum toxin type A is daxibotulinumtoxinA. In another embodiment are methods, wherein the skin condition in the subject is acne vulgaris. In another embodiment are methods, wherein the skin condition in the subject is acne rosacea type 1. In another embodiment are methods, wherein the skin condition in the subject is acne rosacea type 2. In another embodiment are methods, wherein the skin condition in the subject is psoriasis. In another embodiment are methods, wherein the skin condition in the subject is hyperhidrosis. In another embodiment are methods, wherein the second composition comprising one or more botulinum toxins is applied topically to the skin of the subject. In another embodiment are methods, wherein the second composition comprising one or more botulinum toxins is in the form of an aqueous solution. In another embodiment are methods, wherein the second composition is applied to the skin of the subject in the form of a solution. In another embodiment are methods, wherein the second composition is in the form of an aqueous solution. In another embodiment are methods, wherein the amount of the first composition comprising Spongilla applied to the skin of the subject is from about 0.5 grams to about 50 grams, measured as a dry weight. In another embodiment are methods, wherein the first composition is applied to the skin of the subject in the form of a paste. In another embodiment are methods, wherein the paste further comprises water or saline. In another embodiment are methods, wherein the paste is prepared by mixing a powder comprising Spongilla and an aqueous solution comprising hydrogen peroxide. In another embodiment are methods, wherein the aqueous solution comprises hydrogen peroxide at a w/w concentration of about 3%. In another embodiment are methods, wherein the first composition is applied to the skin of the subject prior to the second composition being applied to the skin of the subject. In another embodiment are methods, wherein the first composition is applied to the skin of the subject and is permitted to dry on the skin of the subject prior to application of the second composition to the skin of the subject. In another embodiment are methods, wherein the first composition is applied to the skin of the subject in the form of an aqueous paste, wherein the aqueous portion is derived from water or saline. In another embodiment are methods, wherein the aqueous paste further comprises hydrogen peroxide. In another embodiment are methods, wherein an aqueous solution of hydrogen peroxide is applied to the face of the subject following application of the first composition to the skin of the subject and prior to the application of the second composition to the skin of the subject. In another embodiment are methods, wherein the first composition further comprises hydrogen peroxide. In another embodiment are methods, wherein the hydrogen peroxide is an aqueous solution. In another embodiment are methods, wherein the aqueous solution comprises hydrogen peroxide at a w/w concentration of about 3%. In another embodiment are methods, wherein the second composition is permitted to dry on the skin of the subject following application to the skin of the subject. In another embodiment are methods, wherein the second composition is applied to the skin of the subject prior to the first composition being applied to the skin of the subject. In another embodiment are methods, wherein the second composition is permitted to dry on the skin of the subject prior to the first composition being applied to the skin of the subject. In another embodiment are methods, wherein the first composition is applied to the skin of the subject in the form of an aqueous paste, wherein the aqueous portion is derived from water or saline. In another embodiment are methods, wherein the aqueous paste further comprises hydrogen peroxide. In another embodiment are methods, wherein an aqueous solution of hydrogen peroxide is applied to the face of the subject following application of the first composition to the skin of the subject. In another embodiment are methods, wherein the hydrogen peroxide is an aqueous solution. In another embodiment are methods, wherein the aqueous solution comprises hydrogen peroxide at a w/w concentration of about 3%. In another embodiment are methods, wherein the first composition is permitted to dry on the skin of the subject. In another embodiment are methods, wherein the first composition and the second composition are mixed together, and the resulting mixture is applied to the skin of the subject. In another embodiment are methods, wherein the first composition is mixed with an aqueous solution of hydrogen peroxide prior to mixing with the second composition. In another embodiment are methods, wherein the mixture of the first composition and the second composition is further mixed with an aqueous solution of hydrogen peroxide prior to application to the skin of the subject. In another embodiment are methods, wherein the aqueous solution comprises hydrogen peroxide at a w/w concentration of about 3%. In another embodiment are methods, wherein the first composition and the second composition are applied to the skin of the subject once per week. In another embodiment are methods, wherein the second composition is applied to the skin of the subject no more than once every 4 weeks. In another embodiment are methods, wherein the first composition is applied to the skin of the subject at least once per week for at least one week. In another embodiment are methods, wherein the first composition is applied to the skin of the subject once per week for 6 weeks. In another embodiment are methods, wherein the first composition and the second composition are applied to the skin of the subject on at least one of the subject's face, back and chest. In another embodiment are methods, wherein the first composition and the second composition are applied to the skin of the subject on the subject's face. In another embodiment are methods, wherein the first composition and the second composition are applied to the skin of the subject on the subject's back. In another embodiment are methods, wherein the first composition c and the second composition comprising are applied to the skin of the subject on the subject's chest.

In one aspect is provided a method of treating a skin condition in a subject, comprising applying to the skin of the subject a first composition and a second composition, wherein (a) the first composition comprises Spongilla lacustris powder; (b) the second composition comprises one or more botulinum toxin type selected from botulinum toxin type A; and (c) the skin condition in the subject is selected from acne vulgaris, acne rosacea type 1, acne rosacea type 2, psoriasis, and hyperhidrosis. In another embodiment are methods, wherein the second composition comprises onabotulinumtoxinA, and the skin condition in the subject is acne vulgaris. In another embodiment are methods, wherein the second composition comprises onabotulinumtoxinA, and the skin condition in the subject is acne rosacea type 1. In another embodiment are methods, wherein the second composition comprises onabotulinumtoxinA, and the skin condition in the subject is acne rosacea type 2. In another embodiment are methods, wherein the second composition comprises onabotulinumtoxinA, and the skin condition in the subject is psoriasis. In another embodiment are methods, wherein the second composition comprises onabotulinumtoxinA, and the skin condition in the subject is hyperhidrosis. In another embodiment are methods, wherein the second composition comprises abobotulinumtoxinA. In another embodiment are methods, wherein the second composition comprises abobotulinumtoxinA, and the skin condition in the subject is acne vulgaris. In another embodiment are methods, wherein the second composition comprises abobotulinumtoxinA, and the skin condition in the subject is acne rosacea type 1. In another embodiment are methods, wherein the second composition comprises abobotulinumtoxinA, and the skin condition in the subject is acne rosacea type 2. In another embodiment are methods, wherein the second composition comprises abobotulinumtoxinA, and the skin condition in the subject is psoriasis. In another embodiment are methods, wherein the second composition comprises abobotulinumtoxinA, and the skin condition in the subject is hyperhidrosis. In another embodiment are methods, wherein the second composition comprises incobotulinumtoxinA. In another embodiment are methods, wherein the second composition comprises incobotulinumtoxinA, and the skin condition in the subject is acne vulgaris. In another embodiment are methods, wherein the second composition comprises incobotulinumtoxinA, and the skin condition in the subject is acne rosacea type 1. In another embodiment are methods, wherein the second composition comprises incobotulinumtoxinA, and the skin condition in the subject is acne rosacea type 2. In another embodiment are methods, wherein the second composition comprises incobotulinumtoxinA, and the skin condition in the subject is psoriasis. In another embodiment are methods, wherein the second composition comprises incobotulinumtoxinA, and the skin condition in the subject is hyperhidrosis. In another embodiment are methods, wherein the second composition comprises prabotulinumtoxinA. In another embodiment are methods, wherein the second composition comprises prabotulinumtoxinA, and the skin condition in the subject is acne vulgaris. In another embodiment are methods, wherein the second composition comprises prabotulinumtoxinA, and the skin condition in the subject is acne rosacea type 1. In another embodiment are methods, wherein the second composition comprises prabotulinumtoxinA, and the skin condition in the subject is acne rosacea type 2. In another embodiment are methods, wherein the second composition comprises prabotulinumtoxinA, and the skin condition in the subject is psoriasis. In another embodiment are methods, wherein the second composition comprises prabotulinumtoxinA, and the skin condition in the subject is hyperhidrosis. In another embodiment are methods, wherein the second composition comprises daxibotulinumtoxinA. In another embodiment are methods, wherein the second composition comprises daxibotulinumtoxinA, and the skin condition in the subject is acne vulgaris. In another embodiment are methods, wherein the second composition comprises daxibotulinumtoxinA, and the skin condition in the subject is acne rosacea type 1. In another embodiment are methods, wherein the second composition comprises daxibotulinumtoxinA, and the skin condition in the subject is acne rosacea type 2. In another embodiment are methods, wherein the second composition comprises daxibotulinumtoxinA, and the skin condition in the subject is psoriasis. In another embodiment are methods, wherein the second composition comprises daxibotulinumtoxinA, and the skin condition in the subject is hyperhidrosis.

In another embodiment are methods, wherein the second composition comprises EB-001A. In another embodiment are methods, wherein the second composition comprises EB-001A, and the skin condition in the subject is acne vulgaris. In another embodiment are methods, wherein the second composition comprises EB-001A, and the skin condition in the subject is acne rosacea type 1. In another embodiment are methods, wherein the second composition comprises EB-001A, and the skin condition in the subject is acne rosacea type 2. In another embodiment are methods, wherein the second composition comprises EB-001A, and the skin condition in the subject is psoriasis. In another embodiment are methods, wherein the second composition comprises EB-001A, and the skin condition in the subject is hyperhidrosis. In another embodiment are methods, wherein the second composition comprises EB-001T. In another embodiment are methods, wherein the second composition comprises EB-001T, and the skin condition in the subject is acne vulgaris. In another embodiment are methods, wherein the second composition comprises EB-001T, and the skin condition in the subject is acne rosacea type 1. In another embodiment are methods, wherein the second composition comprises EB-001T, and the skin condition in the subject is acne rosacea type 2. In another embodiment are methods, wherein the second composition comprises EB-001T, and the skin condition in the subject is psoriasis. In another embodiment are methods, wherein the second composition comprises EB-001T, and the skin condition in the subject is hyperhidrosis.

In another embodiment are methods, wherein the second composition comprising one or more botulinum toxins is applied topically to the skin of the subject. In another embodiment are methods, wherein the second composition comprising one or more botulinum toxins is in the form of an aqueous solution. In another embodiment are methods, wherein the second composition is applied to the skin of the subject in the form of a solution. In another embodiment are methods, wherein the second composition is in the form of an aqueous solution. In another embodiment are methods, wherein the amount of the first composition comprising Spongilla applied to the skin of the subject is from about 0.5 grams to about 50 grams, measured as a dry weight. In another embodiment are methods, wherein the first composition is applied to the skin of the subject in the form of a paste. In another embodiment are methods, wherein the paste further comprises water or saline. In another embodiment are methods, wherein the paste is prepared by mixing a powder comprising Spongilla and an aqueous solution comprising hydrogen peroxide. In another embodiment are methods, wherein the aqueous solution comprises hydrogen peroxide at a w/w concentration of about 3%. In another embodiment are methods, wherein the first composition is applied to the skin of the subject prior to the second composition being applied to the skin of the subject. In another embodiment are methods, wherein the first composition is applied to the skin of the subject and is permitted to dry on the skin of the subject prior to application of the second composition to the skin of the subject. In another embodiment are methods, wherein the first composition is applied to the skin of the subject in the form of an aqueous paste, wherein the aqueous portion is derived from water or saline. In another embodiment are methods, wherein the aqueous paste further comprises hydrogen peroxide. In another embodiment are methods, wherein an aqueous solution of hydrogen peroxide is applied to the face of the subject following application of the first composition to the skin of the subject and prior to the application of the second composition to the skin of the subject. In another embodiment are methods, wherein the first composition further comprises hydrogen peroxide. In another embodiment are methods, wherein the hydrogen peroxide is an aqueous solution. In another embodiment are methods, wherein the aqueous solution comprises hydrogen peroxide at a w/w concentration of about 3%. In another embodiment are methods, wherein the second composition is permitted to dry on the skin of the subject following application to the skin of the subject. In another embodiment are methods, wherein the second composition is applied to the skin of the subject prior to the first composition being applied to the skin of the subject. In another embodiment are methods, wherein the second composition is permitted to dry on the skin of the subject prior to the first composition being applied to the skin of the subject. In another embodiment are methods, wherein the first composition is applied to the skin of the subject in the form of an aqueous paste, wherein the aqueous portion is derived from water or saline. In another embodiment are methods, wherein the aqueous paste further comprises hydrogen peroxide. In another embodiment are methods, wherein an aqueous solution of hydrogen peroxide is applied to the face of the subject following application of the first composition to the skin of the subject. In another embodiment are methods, wherein the hydrogen peroxide is an aqueous solution. In another embodiment are methods, wherein the aqueous solution comprises hydrogen peroxide at a w/w concentration of about 3%. In another embodiment are methods, wherein the first composition is permitted to dry on the skin of the subject. In another embodiment are methods, wherein the first composition and the second composition are mixed together, and the resulting mixture is applied to the skin of the subject. In another embodiment are methods, wherein the first composition is mixed with an aqueous solution of hydrogen peroxide prior to mixing with the second composition. In another embodiment are methods, wherein the mixture of the first composition and the second composition is further mixed with an aqueous solution of hydrogen peroxide prior to application to the skin of the subject. In another embodiment are methods, wherein the aqueous solution comprises hydrogen peroxide at a w/w concentration of about 3%.

In another aspect is provided a kit, comprising a first composition and a second composition, wherein (a) the first composition comprises a Spongilla; and (b) the second composition comprises one or more botulinum toxins. In another aspect is provided any of the kits described herein, further comprising instructions for use in treating the first and the second composition in the treatment in a subject having a skin condition. In another aspect is provided any of the kits described herein, wherein the first composition comprises Spongilla in the form of a powder. In another aspect is provided any of the kits described herein, wherein the Spongilla is in the form of a powder comprising particles that are substantially uniform in size.

In another aspect is provided any of the kits described herein, wherein not less than 50% of the particles comprising the Spongilla powder pass through a US 70-mesh screen.

In another aspect is provided any of the kits described herein, wherein not less than about 50% of the particles comprising the Spongilla powder pass through a US 70-mesh screen. In another embodiment are methods, wherein not less than about 60%, or about 70%, or about 75%, or about 80%, or about 85%, or about 90%, or about 95%, or about 96%, or about 97%, or about 98%, or about 99% of the particles comprising the Spongilla powder pass through a US 70-mesh screen. In another aspect is provided any of the kits disclosed herein, wherein not less than about 95%, or about 96%, or about 97%, or about 98%, or about 99% of the particles comprising the Spongilla powder pass through a US 70-mesh screen. In another aspect is provided any of the kits disclosed herein, wherein not less than about 95% of the particles comprising the Spongilla powder pass through a US 70-mesh screen. In another aspect is provided any of the kits disclosed herein, wherein not less than about 96% of the particles comprising the Spongilla powder pass through a US 70-mesh screen. In another aspect is provided any of the kits disclosed herein, wherein not less than about 97% of the particles comprising the Spongilla powder pass through a US 70-mesh screen. In another aspect is provided any of the kits disclosed herein, wherein not less than about 98% of the particles comprising the Spongilla powder pass through a US 70-mesh screen. In another aspect is provided any of the kits disclosed herein, wherein not less than about 99% of the particles comprising the Spongilla powder pass through a US 70-mesh screen.

In another aspect is provided any of the kits disclosed herein, wherein the particles comprising the Spongilla powder have an average length of from about 50 μm to about 500 μm. In another aspect is provided any of the kits disclosed herein, wherein the particles comprising the Spongilla powder have an average length of from about 50 μm to about 400 μm, or from about 50 μm to about 350 μm, or from about 50 μm to about 300 μm, or from about 50 μm to about 250 μm, or from about 50 μm to about 200 μm, or from about 75 μm to about 500 μm, or from about 75 μm to about 450 μm, or from about 80 μm to about 450 μm, or from about 80 μm to about 400 μm, or from about 85 μm to about 450 μm, or from about 85 μm to about 400 μm, or from about 90 μm to about 450 μm, or from about 90 μm to about 400 μm, or from about 90 μm to about 350 μm, or from about 100 μm to about 450 μm, or from about 100 μm to about 400 μm, or from about 100 μm to about 350 μm, or from about 100 μm to about 300 μm, or from about 100 μm to about 250 μm, or from about 100 μm to about 200 μm, or from about 150 μm to about 500 μm, or from about 150 μm to about 450 μm, or from about 150 μm to about 400 μm, or from about 150 μm to about 350 μm, or from about 150 μm to about 350 μm, or from about 150 μm to about 300 μm, or from about 150 μm to about 250 μm, or from about 150 μm to about 200 μm, or from about 175 μm to about 450 μm, or from about 175 μm to about 400 μm, or from about 175 μm to about 350 μm, or from about 175 μm to about 300 μm, or from about 175 μm to about 250 μm, or from about 175 μm to about 200 μm. In another aspect is provided any of the kits disclosed herein, wherein the particles comprising the Spongilla powder have an average length of about 50 μm, or about 75 μm, or about 80 μm, or about 85 μm, or about 90 μm, or about 100 μm, or about 125 μm, or about 150 μm, or about 175 μm, or about 200 μm, or about 225 μm, or about 250 μm, or about 300 μm, or about 350 μm, or about 400 μm, or about 450 μm, or about 500 μm. In another aspect is provided any of the kits disclosed herein, wherein the particles comprising the Spongilla powder have an average length of about 200 μm.

In another aspect is provided any of the kits disclosed herein, wherein the particles comprising the Spongilla powder have an average diameter of from about 5 μm to about 50 μm. In another aspect is provided any of the kits disclosed herein, wherein the particles comprising the Spongilla powder have an average diameter of from about 5 μm to about 45 μm, or from about 5 μm to about 40 μm, from about 5 μm to about 35 μm, from about 5 μm to about 30 μm, from about 5 μm to about 25 μm, from about 5 μm to about 20 μm, from about 10 μm to about 50 μm, from about 10 μm to about 45 μm, from about 10 μm to about 40 μm, from about 10 μm to about 35 μm, from about 10 μm to about 30 μm, from about 10 μm to about 25 μm, from about 10 μm to about 20 μm. In another aspect is provided any of the kits disclosed herein, wherein the particles comprising the Spongilla powder have an average diameter of about 5 μm, or about 10 μm, or about 15 μm, or about 20 μm, or about 25 μm, or about 30 μm, or about 35 μm, or about 40 μm, or about 45 μm, or about 50 μm.

In another aspect is provided any of the kits disclosed herein, wherein the particles comprising the Spongilla powder have an aspect ratio of from about 1 to about 100. In another aspect is provided any of the kits disclosed herein, wherein the particles comprising the Spongilla powder have an aspect ratio of from about 1 to about 75, or from about 1 to about 50, or from about 1 to about 25, or from about 1 to about 20, or from about 1 to about 15, or from about 5 to about 100, or from about 5 to about 75, or from about 5 to about 50, or from about 5 to about 40, or from about 5 to about 35, or from about 5 to about 30, or from about 5 to about 25, or from about 5 to about 20, or from about 5 to about 15, or from about 7 to about 50, or from about 7 to about 45, or from about 7 to about 40, or from about 7 to about 35, or from about 7 to about 30, or from about 7 to about 25, or from about 10 to about 50, or from about 10 to about 45, or from about 10 to about 40, or from about 10 to about 35, or from about 10 to about 30, or from about 10 to about 25, or from about 10 to about 15. In another aspect is provided any of the kits disclosed herein, wherein the particles comprising the Spongilla powder have an aspect ratio of about 5, or about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 35, or about 40, or about 45, or about 50, or about 75, or about 100.

In another aspect is provided any of the kits disclosed herein, wherein the first composition has a residual moisture content of not more than about 20%. In another aspect is provided any of the kits disclosed herein, wherein the first composition has a residual moisture content of not more than about 15%, or not more than about 10%, or not more than about 9%, or not more than about 8%, or not more than about 7%, or not more than about 6%, or not more than about 5%, or not more than about 4%, or not more than about 3%, or not more than about 2%, or not more than 1%. In another aspect is provided any of the kits disclosed herein, wherein the first composition has a residual moisture content of not more than about 5%. In another aspect is provided any of the kits disclosed herein, wherein the first composition has a residual moisture content of not more than about 4%. In another aspect is provided any of the kits disclosed herein, wherein the first composition has a residual moisture content of not more than about 3%. In another aspect is provided any of the kits disclosed herein, wherein the first composition has a residual moisture content of not more than about 2%. In another aspect is provided any of the kits disclosed herein, wherein the first composition has a residual moisture content of not more than about 1%.

In another aspect is provided any of the kits disclosed herein, wherein the first composition has a combined aerobic and anaerobic microbial content of not more than about 25×104 colony-forming units per gram (CFU/g). In another aspect is provided any of the kits disclosed herein, wherein the first composition has a combined aerobic and anaerobic microbial content of not more than about 10×10⁴ CFU/g, or not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the kits disclosed herein, wherein the first composition has a combined aerobic and anaerobic microbial content of not more than about 1,000 CFU/g. In another aspect is provided any of the kits disclosed herein, wherein the first composition has a combined aerobic and anaerobic microbial content of not more than about 750 CFU/g. In another aspect is provided any of the kits disclosed herein, wherein the first composition has a combined aerobic and anaerobic microbial content of not more than about 500 CFU/g. In another aspect is provided any of the kits disclosed herein, wherein the first composition has a combined aerobic and anaerobic microbial content of not more than about 250 CFU/g. In another aspect is provided any of the kits disclosed herein, wherein the first composition has a combined aerobic and anaerobic microbial content of not more than about 200 CFU/g. In another aspect is provided any of the kits disclosed herein, wherein the first composition has a combined aerobic and anaerobic microbial content of not more than about 150 CFU/g. In another aspect is provided any of the kits disclosed herein, wherein the first composition has a combined aerobic and anaerobic microbial content of not more than about 100 CFU/g. In another aspect is provided any of the kits disclosed herein, wherein the first composition has a combined aerobic and anaerobic microbial content of not more than about 75 CFU/g. In another aspect is provided any of the kits disclosed herein, wherein the first composition has a combined aerobic and anaerobic microbial content of not more than about 50 CFU/g. In another aspect is provided any of the kits disclosed herein, wherein the first composition has a combined aerobic and anaerobic microbial content of not more than about 25 CFU/g. In another aspect is provided any of the kits disclosed herein, wherein the first composition has a combined aerobic and anaerobic microbial content of not more than about 20 CFU/g. In another aspect is provided any of the kits disclosed herein, wherein the first composition has a combined aerobic and anaerobic microbial content of not more than about 10 CFU/g. In another aspect is provided any of the kits disclosed herein, wherein the first composition has a combined aerobic and anaerobic microbial content of not more than about 1 CFU/g. In another aspect is provided any of the kits disclosed herein, wherein the first composition has a combined aerobic and anaerobic microbial content of not more than about 25×10⁴ colony-forming units per gram (CFU/g). In another aspect is provided any of the kits disclosed herein, wherein the first composition has a combined aerobic and anaerobic microbial content of not more than about 10×10⁴ CFU/g, or not more than about 5×10⁴ CFU/g, or not more than about 1×10⁴ CFU/g, or not more than about 5×10³ CFU/g, or not more than about 1×10³ CFU/g.

In another aspect is provided any of the kits disclosed herein, wherein the first composition has a combined yeast and mold content of not more than about 25×10⁴ colony-forming units per gram (CFU/g). In another aspect is provided any of the kits disclosed herein, wherein the first composition has a combined yeast and mold content of not more than about or not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the kits disclosed herein, wherein the first composition has a combined yeast and mold content of not more than about 1,000 CFU/g. In another aspect is provided any of the kits disclosed herein, wherein the first composition has a combined yeast and mold content of not more than about 750 CFU/g. In another aspect is provided any of the kits disclosed herein, wherein the first composition has a combined yeast and mold content of not more than about 500 CFU/g. In another aspect is provided any of the kits disclosed herein, wherein the first composition has a combined yeast and mold content of not more than about 250 CFU/g. In another aspect is provided any of the kits disclosed herein, wherein the first composition has a combined yeast and mold content of not more than about 200 CFU/g. In another aspect is provided any of the kits disclosed herein, wherein the first composition has a combined yeast and mold content of not more than about 150 CFU/g. In another aspect is provided any of the kits disclosed herein, wherein the first composition has a combined yeast and mold content of not more than about 100 CFU/g. In another aspect is provided any of the kits disclosed herein, wherein the first composition has a combined yeast and mold content of not more than about 75 CFU/g. In another aspect is provided any of the kits disclosed herein, wherein the first composition has a combined yeast and mold content of not more than about 50 CFU/g. In another aspect is provided any of the kits disclosed herein, wherein the first composition has a combined yeast and mold content of not more than about 25 CFU/g. In another aspect is provided any of the kits disclosed herein, wherein the first composition has a combined yeast and mold content of not more than about 20 CFU/g. In another aspect is provided any of the kits disclosed herein, wherein the first composition has a combined yeast and mold content of not more than about 10 CFU/g. In another aspect is provided any of the kits disclosed herein, wherein the first composition has a combined yeast and mold content of not more than about 1 CFU/g. In another aspect is provided any of the kits disclosed herein, wherein the first composition has a combined yeast and mold content of not more than about 25×10⁴ colony-forming units per gram (CFU/g). In another aspect is provided any of the kits disclosed herein, wherein the first composition has a combined yeast and mold content of not more than about or not more than about 5×10⁴ CFU/g, or not more than about 1×10⁴ CFU/g, or not more than about 5×10³ CFU/g, or not more than about 1×10³ CFU/g.

In another aspect is provided any of the kits disclosed herein, wherein the amount of Coliform bacteria in the first composition is not more than about 25×10⁴ colony-forming units per gram (CFU/g). In another aspect is provided any of the kits disclosed herein, wherein the amount of Coliform bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the kits disclosed herein, wherein the first composition has a Coliform bacteria content of not more than about 1,000 CFU/g. In another aspect is provided any of the kits disclosed herein, wherein the first composition has a Coliform bacteria content of not more than about 750 CFU/g. In another aspect is provided any of the kits disclosed herein, wherein the first composition has a Coliform bacteria content of not more than about 500 CFU/g. In another aspect is provided any of the kits disclosed herein, wherein the first composition has a Coliform bacteria content of not more than about 250 CFU/g. In another aspect is provided any of the kits disclosed herein, wherein the first composition has a Coliform bacteria content of not more than about 200 CFU/g. In another aspect is provided any of the kits disclosed herein, wherein the first composition has a Coliform bacteria content of not more than about 150 CFU/g. In another aspect is provided any of the kits disclosed herein, wherein the first composition has a Coliform bacteria content of not more than about 100 CFU/g. In another aspect is provided any of the kits disclosed herein, wherein the first composition has a Coliform bacteria content of not more than about 75 CFU/g. In another aspect is provided any of the kits disclosed herein, wherein the first composition has a Coliform bacteria content of not more than about 50 CFU/g. In another aspect is provided any of the kits disclosed herein, wherein the first composition has a Coliform bacteria content of not more than about 25 CFU/g. In another aspect is provided any of the kits disclosed herein, wherein the first composition has a Coliform bacteria content of not more than about 20 CFU/g. In another aspect is provided any of the kits disclosed herein, wherein the first composition has a Coliform bacteria content of not more than about 10 CFU/g. In another aspect is provided any of the kits disclosed herein, wherein the first composition has a Coliform bacteria content of not more than about 1 CFU/g. In another aspect is provided any of the kits disclosed herein, wherein the first composition has no detectable Coliform bacterial content. In another aspect is provided any of the kits disclosed herein, wherein the amount of Coliform bacteria in the first composition is not more than about 25×10⁴ colony-forming units per gram (CFU/g). In another aspect is provided any of the kits disclosed herein, wherein the amount of Coliform bacteria in the first composition is not more than about 5×10⁴ CFU/g, or not more than about 1×10⁴ CFU/g, or not more than about 5×10³ CFU/g, or not more than about 1×10³ CFU/g

In another aspect is provided any of the kits disclosed herein, wherein the amount of Salmonella in the first composition is not more than about 25×10⁴ colony-forming units per gram (CFU/g). In another aspect is provided any of the kits disclosed herein, wherein the amount of Salmonella in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the kits disclosed herein, wherein the first composition has a Salmonella content of not more than about 1,000 CFU/g. In another aspect is provided any of the kits disclosed herein, wherein the first composition has a Salmonella content of not more than about 750 CFU/g. In another aspect is provided any of the kits disclosed herein, wherein the first composition has a Salmonella content of not more than about 500 CFU/g. In another aspect is provided any of the kits disclosed herein, wherein the first composition has a Salmonella content of not more than about 250 CFU/g. In another aspect is provided any of the kits disclosed herein, wherein the first composition has a Salmonella content of not more than about 200 CFU/g. In another aspect is provided any of the kits disclosed herein, wherein the first composition has a Salmonella content of not more than about 150 CFU/g. In another aspect is provided any of the kits disclosed herein, wherein the first composition has a Salmonella content of not more than about 100 CFU/g. In another aspect is provided any of the kits disclosed herein, wherein the first composition has a Salmonella content of not more than about 75 CFU/g. In another aspect is provided any of the kits disclosed herein, wherein the first composition has a Salmonella content of not more than about 50 CFU/g. In another aspect is provided any of the kits disclosed herein, wherein the first composition has a Salmonella content of not more than about 25 CFU/g. In another aspect is provided any of the kits disclosed herein, wherein the first composition has a Salmonella content of not more than about 20 CFU/g. In another aspect is provided any of the kits disclosed herein, wherein the first composition has a Salmonella content of not more than about 10 CFU/g. In another aspect is provided any of the kits disclosed herein, wherein the first composition has a Salmonella content of not more than about 1 CFU/g. In another aspect is provided any of the kits disclosed herein, wherein the first composition has no detectable Salmonella content. In another aspect is provided any of the kits disclosed herein, wherein the amount of Salmonella in the first composition is not more than about 25×10⁴ colony-forming units per gram (CFU/g). In another aspect is provided any of the kits disclosed herein, wherein the amount of Salmonella in the first composition is not more than about 5×10⁴ CFU/g, or not more than about 1×10⁴ CFU/g, or not more than about 5×10³ CFU/g, or not more than about 1×10³ CFU/g.

In another aspect is provided any of the kits disclosed herein, wherein the amount of Pseudomonas aeruginosa bacteria in the first composition is not more than about 25×10⁴ colony-forming units per gram (CFU/g). In another aspect is provided any of the kits disclosed herein, wherein the amount of Pseudomonas aeruginosa bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the kits disclosed herein, wherein the first composition has a Pseudomonas aeruginosa bacteria content of not more than about 1,000 CFU/g. In another aspect is provided any of the kits disclosed herein, wherein the first composition has a Pseudomonas aeruginosa bacteria content of not more than about 750 CFU/g. In another aspect is provided any of the kits disclosed herein, wherein the first composition has a Pseudomonas aeruginosa bacteria content of not more than about 500 CFU/g. In another aspect is provided any of the kits disclosed herein, wherein the first composition has a Pseudomonas aeruginosa bacteria content of not more than about 250 CFU/g. In another aspect is provided any of the kits disclosed herein, wherein the first composition has a Pseudomonas aeruginosa bacteria content of not more than about 200 CFU/g. In another aspect is provided any of the kits disclosed herein, wherein the first composition has a Pseudomonas aeruginosa bacteria content of not more than about 150 CFU/g. In another aspect is provided any of the kits disclosed herein, wherein the first composition has a Pseudomonas aeruginosa bacteria content of not more than about 100 CFU/g. In another aspect is provided any of the kits disclosed herein, wherein the first composition has a Pseudomonas aeruginosa bacteria content of not more than about 75 CFU/g. In another aspect is provided any of the kits disclosed herein, wherein the first composition has a Pseudomonas aeruginosa bacteria content of not more than about 50 CFU/g. In another aspect is provided any of the kits disclosed herein, wherein the first composition has a Pseudomonas aeruginosa bacteria content of not more than about 25 CFU/g. In another aspect is provided any of the kits disclosed herein, wherein the first composition has a Pseudomonas aeruginosa bacteria content of not more than about 20 CFU/g. In another aspect is provided any of the kits disclosed herein, wherein the first composition has a Pseudomonas aeruginosa bacteria content of not more than about 10 CFU/g. In another aspect is provided any of the kits disclosed herein, wherein the first composition has a Pseudomonas aeruginosa bacteria content of not more than about 1 CFU/g. In another aspect is provided any of the kits disclosed herein, wherein the first composition has no detectable Pseudomonas aeruginosa bacteria content. In another aspect is provided any of the kits disclosed herein, wherein the amount of Pseudomonas aeruginosa bacteria in the first composition is not more than about 25×10⁴ colony-forming units per gram (CFU/g). In another aspect is provided any of the kits disclosed herein, wherein the amount of Pseudomonas aeruginosa bacteria in the first composition is not more than about 5×10⁴ CFU/g, or not more than about 1×10⁴ CFU/g, or not more than about 5×10³ CFU/g, or not more than about 1×10³ CFU/g.

In another aspect is provided any of the kits disclosed herein, wherein the amount of Staphylococcus aureus bacteria in the first composition is not more than about 25×10⁴ colony-forming units per gram (CFU/g). In another aspect is provided any of the kits disclosed herein, wherein the amount of Staphylococcus aureus bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the kits disclosed herein, wherein the first composition has a Staphylococcus aureus bacteria content of not more than about 1,000 CFU/g. In another aspect is provided any of the kits disclosed herein, wherein the first composition has a Staphylococcus aureus bacteria content of not more than about 750 CFU/g. In another aspect is provided any of the kits disclosed herein, wherein the first composition has a Staphylococcus aureus bacteria content of not more than about 500 CFU/g. In another aspect is provided any of the kits disclosed herein, wherein the first composition has a Staphylococcus aureus bacteria content of not more than about 250 CFU/g. In another aspect is provided any of the kits disclosed herein, wherein the first composition has a Staphylococcus aureus bacteria content of not more than about 200 CFU/g. In another aspect is provided any of the kits disclosed herein, wherein the first composition has a Staphylococcus aureus bacteria content of not more than about 150 CFU/g. In another aspect is provided any of the kits disclosed herein, wherein the first composition has a Staphylococcus aureus bacteria content of not more than about 100 CFU/g. In another aspect is provided any of the kits disclosed herein, wherein the first composition has a Staphylococcus aureus bacteria content of not more than about 75 CFU/g. In another aspect is provided any of the kits disclosed herein, wherein the first composition has a Staphylococcus aureus bacteria content of not more than about 50 CFU/g. In another aspect is provided any of the kits disclosed herein, wherein the first composition has a Staphylococcus aureus bacteria content of not more than about 25 CFU/g. In another aspect is provided any of the kits disclosed herein, wherein the first composition has a Staphylococcus aureus bacteria content of not more than about 20 CFU/g. In another aspect is provided any of the kits disclosed herein, wherein the first composition has a Staphylococcus aureus bacteria content of not more than about 10 CFU/g. In another aspect is provided any of the kits disclosed herein, wherein the first composition has a Staphylococcus aureus bacteria content of not more than about 1 CFU/g. In another aspect is provided any of the kits disclosed herein, wherein the first composition has no detectable Staphylococcus aureus bacteria content. In another aspect is provided any of the kits disclosed herein, wherein the amount of Staphylococcus aureus bacteria in the first composition is not more than about 25×10⁴ colony-forming units per gram (CFU/g). In another aspect is provided any of the kits disclosed herein, wherein the amount of Staphylococcus aureus bacteria in the first composition is not more than about 5×10⁴ CFU/g, or not more than about 1×10⁴ CFU/g, or not more than about 5×10³ CFU/g, or not more than about 1×10³ CFU/g.

In another aspect is provided any of the kits disclosed herein, wherein the first composition is packaged prior to use. In another aspect is provided any of the kits disclosed herein, wherein the first composition is prepared by heating to at least about 70° C. prior to being packaged. In another aspect is provided any of the kits disclosed herein, wherein the first composition is prepared by heating to at least about 50° C., or at least about 60° C., or at least about 75° C., or at least about 80° C., or at least about 85° C., or at least about 90° C., or at least about 100° C., or at least about 110° C., or at least about 115° C., or at least about 120° C., or at least about 125° C., or at least about 130° C., or at least about 135° C., or at least about 140° C., or at least about 150° C., or at least about 160° C., or at least about 170° C., or at least about 180° C., or at least about 190° C., or at least about 200° C. prior to being packaged.

In another aspect is provided any of the kits disclosed herein, wherein the first composition is heated to at least about 70° C. for at least about 5 minutes prior being packaged. In another aspect is provided any of the kits disclosed herein, wherein the first composition is heated to at least about 70° C. for at least about 10 minutes, or at least about 15 minutes, or at least about 20 minutes, or at least about 25 minutes, or at least about 30 minutes, or at least about 35 minutes, or at least about 40 minutes, or at least about 45 minutes, or at least about 50 minutes, or at least about 55 minutes, or at least about 60 minutes, or at least about 75 minutes, or at least about 90 minutes, or at least about 120 minutes, or at least about 180 minutes, or at least about 4 hours, or at least about 5 hours, or at least about 6 hours, or at least about 7 hours, or at least about 8 hours, or at least about 9 hours, or at least about 10 hours, or at least about 11 hours, or at least about 12 hours, or at least about 24 hours prior being packaged.

In another aspect is provided any of the kits disclosed herein, wherein the first composition is prepared by treatment with ionizing radiation, such as gamma radiation, prior to being packaged or after packaging. For example, gamma irradiation may be performed on the raw Spongilla material prior to grinding to reduce the particle size, following grinding to reduce the particle size, the materials packaged in bulk and or the materials following packaging in unit dose containers. The materials comprising the kits disclosed herein may be treated with ionizing radiation, such as gamma radiation, using methods and equipment known to those of ordinary skill in the art, such as gamma irradiators or electron beam irradiators. In another aspect is provided any of the kits disclosed herein, wherein the first composition is prepared by treating with ionizing radiation, such as gamma radiation, to deliver an absorbed radiation dose between about 1 kGy and about 50 kGy prior to being packaged. In another aspect is provided any of the kits disclosed herein, wherein the first composition is prepared by treating with ionizing radiation, such as gamma radiation, to deliver an absorbed radiation dose between about 1 kGy and about 45 kGy, or between about 1 kGy and about 40 kGy, between about 1 kGy and about 35 kGy, between about 1 kGy and about 30 kGy, or between about 1 kGy and about 25 kGy or between about 5 kGy and about 50 kGy, or between about 5 kGy and about 45 kGy, or between about 5 kGy and about 40 kGy, or between about 5 kGy and about 35 kGy, or between about 5 kGy and about 30 kGy, or between about 5 kGy and about 25 kGy, or between about 10 kGy and about 50 kGy, or between about 10 kGy and about 45 kGy, or between about 10 kGy and about 40 kGy, or between about 10 kGy and about 35 kGy, or between about 10 kGy and about 30 kGy, or between about 10 kGy and about 25 kGy, or between about 15 kGy and about 50 kGy, or between about 15 kGy and about 45 kGy, or between about 15 kGy and about 40 kGy, or between about 15 kGy and about 35 kGy, or between about 15 kGy and about 30 kGy, or between about 15 kGy and about 25 kGy. In another aspect is provided any of the methods disclosed herein, wherein the first composition is prepared by treating with ionizing radiation, such as gamma radiation, to deliver an absorbed radiation dose of about 1 kGy, or about 5 kGy, or about 10 kGy, 11 kGy, or about 12 kGy, or about 13 kGy, or about 14 kGy, or about 15 kGy, or about 16 kGy, or about 17 kGy, or about 18 kGy, or about 19 kGy, or about 20 kGy, or about 21 kGy, or about 22 kGy, or about 23 kGy, or about 24 kGy, or about 25 kGy, or about 26 kGy, or about 27 kGy, or about 28 kGy, or about 29 kGy, or about 30 kGy, or about 31 kGy, or about 32 kGy, or about 33 kGy, or about 34 kGy, or about 35 kGy, or about 36 kGy, or about 37 kGy, or about 38 kGy, or about 39 kGy, or about 40 kGy, or about 41 kGy, or about 42 kGy, or about 43 kGy, or about 44 kGy, or about 45 kGy, or about 46 kGy, or about 47 kGy, or about 48 kGy, or about 49 kGy, or about 50 kGy.

In another aspect is provided any of the kits disclosed herein, wherein the first composition further comprises an aqueous solution of hydrogen peroxide. In another aspect is provided any of the kits disclosed herein, wherein the hydrogen peroxide is at a concentration of from about 0.1% w/w to about 50% w/w, or from about 0.1% w/w to about 45% w/w, or from about 0.1% w/w to about 40% w/w, or from about 0.1% w/w to about 35% w/w, or from about 0.1% w/w to about 30% w/w, or from about 0.1% w/w to about 25% w/w, or from about 0.1% w/w to about 20% w/w, or from about 0.1% w/w to about 15% w/w, or from about 0.1% w/w to about 10% w/w, or from about 0.1% w/w to about 9% w/w, or from about 0.1% w/w to about 8% w/w, or from about 0.1% w/w to about 7% w/w, or from about 0.1% w/w to about 6% w/w, or from about 0.1% w/w to about 5% w/w, or from about 0.1% w/w to about 4% w/w, or from about 0.1% w/w to about 3% w/w, or from about 0.1% w/w to about 2% w/w, or from about 0.1% w/w to about 1% w/w, or from about 0.5% w/w to about 45% w/w, or from about 1% w/w to about 45% w/w, or from about 1% w/w to about 40% w/w, or from about 1% w/w to about 35% w/w, or from about 1% w/w to about 30% w/w, or from about 1% w/w to about 25% w/w, or from about 1% w/w to about 20% w/w, or from about 1% w/w to about 15% w/w, or from about 1% w/w to about 10% w/w, or from about 1% w/w to about 9% w/w, or from about 1% w/w to about 8% w/w, or from about 1% w/w to about 7% w/w, or from about 1% w/w to about 6% w/w, or from about 1% w/w to about 5% w/w, or from about 1% w/w to about 4% w/w, or from about 1% w/w to about 3% w/w, or from about 1% w/w to about 2% w/w, or from about 2% w/w to about 45% w/w, or from about 2% w/w to about 40% w/w, or from about 2% w/w to about 35% w/w, or from about 2% w/w to about 30% w/w, or from about 2% w/w to about 25% w/w, or from about 2% w/w to about 20% w/w, or from about 2% w/w to about 15% w/w, or from about 2% w/w to about 10% w/w, or from about 2% w/w to about 9% w/w, or from about 2% w/w to about 8% w/w, or from about 1% w/w to about 7% w/w, or from about 2% w/w to about 6% w/w, or from about 2% w/w to about 5% w/w, or from about 2% w/w to about 4% w/w, or from about 2% w/w to about 3% w/w. In another aspect is provided any of the kits disclosed herein, wherein the hydrogen peroxide is at a concentration of about 0.1% w/w, or about 0.5% w/w, or about 1% w/w, or about 2% w/w, or about 3% w/w, or about 4% w/w, or about 5% w/w, or about 6% w/w, or about 7% w/w, or about 8% w/w, or about 9% w/w, or about 10% w/w, or about 15% w/w, or about 20% w/w, or about 25% w/w, or about 30% w/w, or about 35% w/w, or about 40% w/w, or about 45% w/w, or about 50% w/w. In another aspect is provided any of the methods disclosed herein, wherein the hydrogen peroxide is at a concentration of about 3% w/w. Aqueous hydrogen peroxide solutions that may be useful in treating skin conditions in a subject as disclosed herein are commercially available or may be prepared by methods known to those of ordinary skill in the art.

In another aspect is provided any of the kits disclosed herein, further comprising a gel or cream comprising hydrogen peroxide. Such gels or creams are generally commercially available any may contain from about 0.5% w/w to about 50% w/w hydrogen peroxide. For example, a gel containing about 1% w/w, or about 2% w/w, or about 3% w/w, or about 4% w/w, or about 6% w/w, or about 7% w/w, or about 8% w/w, or about 9% w/w, or about 10% w/w, or about 15% w/w, or about 20% w/w, or about 25% w/w, or about 30% w/w, or about 40% w/w, or about 45% w/w, or about 50% w/w hydrogen peroxide may be used in any of the methods and kits disclosed herein in combination with the first composition and the second composition.

In another aspect is provided any of the kits disclosed herein, wherein the Spongilla is Spongilla lacustris.

In another aspect is provided any of the kits described herein, wherein the second composition comprises one or more botulinum toxin type selected from botulinum toxin type A, botulinum toxin type B, botulinum toxin type C₁, botulinum toxin type C₂, botulinum toxin type D, botulinum toxin type E, botulinum toxin type F and botulinum toxin type G. In another aspect is provided any of the kits described herein, wherein the one or more botulinum toxin type is selected from botulinum toxin type A and botulinum toxin type B. In another aspect is provided any of the kits described herein, wherein the one or more botulinum toxin type is botulinum toxin type A. In another aspect is provided any of the kits described herein, wherein the botulinum toxin type A is selected from onabotulinumtoxinA, abobotulinumtoxinA, incobotulinumtoxinA, prabotulinumtoxinA, and daxibotulinumtoxinA. In another aspect is provided any of the kits described herein, wherein the botulinum toxin type A is onabotulinumtoxinA. In another aspect is provided any of the kits described herein, wherein the botulinum toxin type A is abobotulinumtoxinA. In another aspect is provided any of the kits described herein, wherein the botulinum toxin type A is incobotulinumtoxinA. In another aspect is provided any of the kits described herein, wherein the botulinum toxin type A is prabotulinumtoxinA. In another aspect is provided any of the kits described herein, wherein the botulinum toxin type A is daxibotulinumtoxinA. In another aspect is provided any of the kits described herein, wherein the one or more botulinum toxin type is botulinum toxin type B. In another aspect is provided any of the methods disclosed herein, wherein the one or more botulinum toxin type B is rimabotulinumtoxinB. In another aspect is provided any of the kits described herein, wherein the one or more botulinum toxin type is botulinum toxin type C₁. In another aspect is provided any of the kits described herein, wherein the one or more botulinum toxin type is botulinum toxin type C₂. In another aspect is provided any of the kits described herein, wherein the one or more botulinum toxin type is botulinum toxin type D. In another aspect is provided any of the kits described herein, wherein the one or more botulinum toxin type is botulinum toxin type E.

In another aspect is provided any of the methods disclosed herein, wherein the one or more botulinum toxin type E is EB-001A or EB-001T. In another aspect is provided any of the methods disclosed herein, wherein the one or more botulinum toxin type E is EB-001A. In another aspect is provided any of the methods disclosed herein, wherein the one or more botulinum toxin type E is EB-001T. In another aspect is provided any of the kits described herein, wherein the one or more botulinum toxin type is botulinum toxin type F. In another aspect is provided any of the kits described herein, wherein the one or more botulinum toxin type is botulinum toxin type G.

In another aspect is provided any of the kits described herein, wherein the kit is used for the treatment of a skin condition in a subject. In another aspect is provided any of the kits described herein, wherein the kit is for use in the treatment of a skin condition in a subject. In another aspect is provided any of the kits described herein, wherein the skin condition in the subject is selected from acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma. In another aspect is provided any of the kits described herein, wherein the skin condition in the subject is selected from acne vulgaris, acne rosacea type 1, acne rosacea type 2, psoriasis, and hyperhidrosis. In another aspect is provided any of the kits described herein, wherein the skin condition in the subject is acne vulgaris. In another aspect is provided any of the kits described herein, wherein the skin condition in the subject is acne rosacea type 1. In another aspect is provided any of the kits described herein, wherein the skin condition in the subject is acne rosacea type 2. In another aspect is provided any of the kits described herein, wherein the skin condition in the subject is psoriasis. In another aspect is provided any of the kits described herein, wherein the skin condition in the subject is hyperhidrosis.

In another embodiment are provided methods to treat, reduce the symptoms of, ameliorate the symptoms of, delay the onset of, or otherwise pharmaceutically address acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma.

The compositions disclosed herein, such as the first composition comprising Spongilla, may further comprise one or more conventional pharmaceutical carriers or excipients. Suitable pharmaceutical carriers and excipients include inert diluents, binders (such as starches), fillers (such as colloidal silicon dioxide, sugars, including lactose, sucrose, mannitol, or sorbitol; and cellulose preparations, for example, maize starch, wheat starch, rice starch, potato starch, gelatin, gum, methyl cellulose, hydroxypropylmethyl cellulose, sodium carboxymethylcellulose, or polyvinylpyrrolidone (PVP)), bulking agents, lubricants (such as magnesium stearate, sodium lauryl sulfate and talc), coloring matters or dyes and, if desired, emulsifying agents or suspending agents, together with diluents such as water, saline, ethanol, propylene glycol, glycerin, or combinations thereof.

The pharmaceutical compositions disclosed herein may be in unit dosage forms suitable for single administration of precise dosages. In another aspect is provided any of the methods or kits disclosed herein, wherein the unit dosage forms of the first compositions and/or the second composition are suitable for two administrations, three administrations, four administrations, five administrations, six administrations, seven administrations, eight administrations, nine administrations, 10 administrations, 11 administrations, 12 administrations, 13 administrations, 14 administrations, 15 administrations, 16 administrations, 17 administrations, 18 administrations, 19 administrations, 20 administrations, 21 administrations, 22 administrations, 23 administrations, 24 administrations, 25 administrations, 26 administrations, 27 administrations, 28 administrations, 29 administrations, 30 administrations, administrations for two months, administrations for three months, administrations for four months, administrations for five months, administrations for six months, administrations for seven months, administrations for eight months, administrations for nine months, administrations for ten months, administrations for eleven months, or administrations for 12 months.

It will be appreciated that the actual dosages of the compositions disclosed herein, may vary according to the composition being used, the mode of administration, and the particular site of the subject being treated, and the skin condition being treated. Those skilled in the art using conventional dosage-determination tests in view of the experimental data for a given composition may ascertain optimal dosages for a given set of conditions. This amount will vary depending upon a variety of factors, including but not limited to the characteristics of the compositions and formulations disclosed herein (including activity, pharmacokinetics, pharmacodynamics, and bioavailability thereof), the physiological condition of the subject treated (including age, sex, disease type and stage, general physical condition, responsiveness to a given dosage, and type of medication) or cells, the nature of the pharmaceutically acceptable carrier mg/kg or carriers in the formulation, and the route of administration. Further, an effective or therapeutically effective amount may vary depending on whether the one or more compositions and formulations disclosed herein is administered alone or in combination with other drug(s), other therapy/therapies or other therapeutic method(s) or modality/modalities. One skilled in the clinical and pharmacological arts will be able to determine an effective amount or therapeutically effective amount through routine experimentation, namely by monitoring a cell's or subject's response to administration of the one or more compositions and formulations disclosed herein and adjusting the dosage accordingly.

Dosage regimens using the first composition and the second composition may be adjusted to provide the optimum desired response. Dosage unit form, as used herein, refers to physically discrete units suited as unitary dosages for the subjects to be treated; each unit containing a predetermined quantity of the compositions disclosed herein, calculated to produce the desired therapeutic effect in association with the required pharmaceutical carrier. The specification for the dosage unit forms of the compositions disclosed herein are dictated by and directly dependent on (a) the characteristics of the composition and the particular therapeutic or prophylactic effect to be achieved, and (b) the limitations inherent in the art of compounding such a composition for the treatment a particular condition in a subject.

Thus, the skilled artisan would appreciate, based upon the disclosure provided herein, that the dose and dosing regimen using the compositions disclosed herein may be adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the presently disclosed methods.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. The embodiments disclosed herein are intended to encompass intra-subject dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

In some embodiments, the compositions may be used in combination with one or more additional compositions useful in treating skin conditions in a subject which are described below. When a combination therapy is used, the one or more additional compositions may be administered sequentially or simultaneously with the first composition and/or the second composition disclosed herein. In some embodiments, the additional compositions is administered to a subject prior to, at the same time as, or following administration of the first composition and/or the second composition disclosed herein. In some embodiments, the additional composition is administered to the subject prior to the administration of the first composition and/or the second composition disclosed herein. In some embodiments, the additional composition is administered to the subject at the same time the first composition and/or the second composition disclosed herein are administered to the subject. In some embodiments, the additional composition is administered to the subject following to the administration of the first composition and/or the second composition disclosed herein. Among the additional compositions that may be used according to any of the methods disclosed herein include, but are not limited to, cromolyn sodium (also known as sodium cromoglycate), topical alpha agonists (including, but not limited to, oxymetazoline hydrochloride, clonidine hydrochloride, apraclonidine hydrochloride, and brimonidine tartrate), topical antibiotics (including, but not limited to, tetracyclines [tetracycline, doxycycline, minocycline, sarecycline], clindamycin, and erythromycin), benzoyl peroxide, salicylic acid, azelaic acid, retinoids, topical anticholinergics (including, but not limited to, oxybutynin, glycopyrrolate, propantheline), topical prostaglandin analogs (including, but not limited to, latanoprost, bimatoprost, travoprost, and tafluprost), and topical hydroquinone or a combination of fluocinolone acetonide, hydroquinone, and tretinoin (sold as Tri-Luma® cream).

In another aspect is provided any of the methods disclosed herein, wherein the Spongilla is Spongilla lacustris. In another aspect is provided any of the kits disclosed herein, wherein the Spongilla is Spongilla lacustris.

In another aspect is provided any of the methods, compositions and kits disclosed herein wherein the first composition is derived from one or more sponges. In another aspect, the one or more sponges may be marine sponges or freshwater sponges. In another aspect, the one or more sponges is a marine sponge. In another aspect, the sponge is a freshwater sponge. In another aspect, the compositions are derived from sponges of the phylum Porifera. In another aspect, the compositions are derived from sponges of the class Demospongiae. In another aspect, the compositions are derived from sponges of the order Spongdilla. In another aspect, the compositions are derived from sponges of the family Spongillidae. In another aspect, the compositions are derived from sponges of the genus Spongilla. In another aspect, the compositions are derived from sponges of the species Spongilla lacustris. In another aspect, the compositions are derived from sponges of the order Haplosclerida. In another aspect, the compositions are derived from sponges of the family Chalinidea. In another aspect, the compositions are derived from sponges of the genus Halciona.

As will be understood by one skilled in the art, for all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

The preparations and examples of a number of embodiments disclosed herein are intended to be illustrative and not limiting. All starting materials are available commercially or are described in the literature. All temperatures are reported in ° C.

### Example 1: Regimen 1

Week 1: The subject's skin is washed and dried with a non-comedogenic cleanser. A container comprising botulinum toxin (botulinum toxin type A, botulinum toxin type B, botulinum toxin type C1, botulinum toxin type C2, botulinum toxin type D, botulinum toxin type E, botulinum toxin type F and botulinum toxin type G, onabotulinumtoxinA, abobotulinumtoxinA, incobotulinumtoxinA, prabotulinumtoxinA, daxibotulinumtoxinA, rimabotulinumtoxinB, EB-001A, or EB-001T) is reconstituted with 10 mL of 0.9% sterile saline and the reconstituted solution is set aside. Three-percent (3%) hydrogen peroxide USP 9 (6 mL) is added to 3 grams (dry weight) of Spongilla powder, and the resulting mixture is stirred until thoroughly mixed. The Spongilla mixture is applied to the affected area of the subject's skin taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser. The reconstituted botulinum toxin composition is then applied to the skin of the subject in the affected areas and in the areas in which the Spongilla composition was applied taking care to avoid mucous membranes (e.g., eyes, mouth, and nostrils). The botulinum toxin composition is allowed to dry on the application area and is then cleaned using non-comedogenic wipes or water and a non-comedogenic cleanser.

Weeks 2 through 6: Three-percent (3%) hydrogen peroxide USP (6 mL) are added to 3 grams (dry weight) of Spongilla powder composition and the resulting mixture is stirred until thoroughly mixed. The Spongilla mixture is applied to the affected area of the subject's skin taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser.

Maintenance Regimen: After the 6-week treatment regimen is complete, the subject can apply the composition comprising Spongilla and/or Spongilla and 3% hydrogen peroxide to the affected areas monthly. The application of the composition comprising botulinum toxin may be repeated as indicated by the symptoms of the subject, but no sooner than 3 months since the immediately prior treatment using the composition comprising the botulinum toxin.

### Example 2

Week 1: The subject's skin is washed and dried with a non-comedogenic cleanser. A container comprising botulinum toxin (botulinum toxin type A, botulinum toxin type B, botulinum toxin type C1, botulinum toxin type C2, botulinum toxin type D, botulinum toxin type E, botulinum toxin type F and botulinum toxin type G, onabotulinumtoxinA, abobotulinumtoxinA, incobotulinumtoxinA, prabotulinumtoxinA, daxibotulinumtoxinA, rimabotulinumtoxinB, EB-001A, or EB-001T) is reconstituted with 10 mL of 0.9% sterile saline and the reconstituted solution is set aside. Three percent (3%) hydrogen peroxide USP (6 mL) is added to 3 grams (dry weight) of Spongilla powder, and the resulting mixture is stirred thoroughly mixed. The reconstituted botulinum toxin composition is then applied to the skin of the subject in the affected areas and in the areas in which the Spongilla composition was applied taking care to avoid mucous membranes (e.g., eyes, mouth, and nostrils). The Spongilla mixture is applied to the affected area of the subject's skin taking care to avoid mucous membranes (e.g., eyes, mouth, and nostrils). Additional Spongilla materials may be applied to areas on the subject's skin where lesions and hyperpigmentation are present. The compositions are allowed to dry on subject's skin to which they were applied and is then removed using non-comedogenic wipes or water and a non-comedogenic cleanser.

Weeks 2 through 6: Three percent (3%) hydrogen peroxide USP (6 mL) are added to 3 grams (dry weight) of Spongilla powder composition and the resulting mixture is stirred thoroughly mixed. The Spongilla mixture is applied to the affected area of the subject's skin taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser.

Maintenance Regimen: After the 6-week treatment regimen is complete, the subject can apply the composition comprising Spongilla and/or Spongilla and 3% hydrogen peroxide to the affected areas monthly. The application of the composition comprising botulinum toxin may be repeated as indicated by the symptoms of the subject, but no sooner than 3 months since the immediately prior treatment using the composition comprising the botulinum toxin.

### Example 3

Week 1: The subject's skin is washed and dried with a non-comedogenic cleanser. A container comprising botulinum toxin (botulinum toxin type A, botulinum toxin type B, botulinum toxin type C1, botulinum toxin type C2, botulinum toxin type D, botulinum toxin type E, botulinum toxin type F and botulinum toxin type G, onabotulinumtoxinA, abobotulinumtoxinA, incobotulinumtoxinA, prabotulinumtoxinA, daxibotulinumtoxinA, rimabotulinumtoxinB, EB-001A, or EB-001T) is reconstituted with 10 mL of 0.9% sterile saline and the reconstituted solution is set aside. Three percent (3%) hydrogen peroxide USP (6 mL) is added to 3 grams (dry weight) of Spongilla powder, and the resulting mixture is stirred thoroughly mixed. A portion of the reconstituted botulinum toxin composition is added to the Spongilla mixture and the resulting mixture is applied to the skin of the subject in the affected areas, taking care to avoid mucous membranes (e.g., eyes, mouth, and nostrils). The compositions are allowed to dry on subject's skin to which they were applied and is then removed using non-comedogenic wipes or water and a non-comedogenic cleanser.

Weeks 2 through 6: Three percent (3%) hydrogen peroxide USP (6 mL) are added to 3 grams (dry weight) of Spongilla powder composition and the resulting mixture is stirred thoroughly mixed. The Spongilla mixture is applied to the affected area of the subject's skin taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser.

Maintenance Regimen: After the 6-week treatment regimen is complete, the subject can apply the composition comprising Spongilla and/or Spongilla and 3% hydrogen peroxide to the affected areas monthly. The application of the composition comprising botulinum toxin may be repeated as indicated by the symptoms of the subject, but no sooner than 3 months since the immediately prior treatment using the composition comprising the botulinum toxin.

### Example 4

Week 1: The subject's skin is washed and dried with a non-comedogenic cleanser. A container comprising botulinum toxin (botulinum toxin type A, botulinum toxin type B, botulinum toxin type C1, botulinum toxin type C2, botulinum toxin type D, botulinum toxin type E, botulinum toxin type F and botulinum toxin type G, onabotulinumtoxinA, abobotulinumtoxinA, incobotulinumtoxinA, prabotulinumtoxinA, daxibotulinumtoxinA, rimabotulinumtoxinB, EB-001A, or EB-001T) is reconstituted with 10 mL of 0.9% sterile saline and the reconstituted solution is set aside. A portion of the reconstituted botulinum toxin composition is added to the Spongilla mixture and the resulting mixture is applied to the to the skin of the subject in the affected areas, taking care to avoid mucous membranes (e.g., eyes, mouth, and nostrils). The compositions are allowed to dry on subject's skin to which they were applied and is then removed using non-comedogenic wipes or water and a non-comedogenic cleanser.

Weeks 2 through 6: Three percent (3%) hydrogen peroxide USP (6 mL) are added to 3 grams (dry weight) of Spongilla powder composition and the resulting mixture is stirred thoroughly mixed. The Spongilla mixture is applied to the affected area of the subject's skin taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser.

Maintenance Regimen: After the 6-week treatment regimen is complete, the subject can apply the composition comprising Spongilla and/or Spongilla and 3% hydrogen peroxide to the affected areas monthly. The application of the composition comprising botulinum toxin may be repeated as indicated by the symptoms of the subject, but no sooner than 3 months since the immediately prior treatment using the composition comprising the botulinum toxin.

### Example 5

Week 1: The subject's skin is washed and dried with a non-comedogenic cleanser. A container comprising botulinum toxin (botulinum toxin type A, botulinum toxin type B, botulinum toxin type C1, botulinum toxin type C2, botulinum toxin type D, botulinum toxin type E, botulinum toxin type F and botulinum toxin type G, onabotulinumtoxinA, abobotulinumtoxinA, incobotulinumtoxinA, prabotulinumtoxinA, daxibotulinumtoxinA, rimabotulinumtoxinB, EB-001A, or EB-001T) is reconstituted with 10 mL of 0.9% sterile saline and the reconstituted solution is set aside. A composition comprising Spongilla powder is applied to the affected area of the subject's skin taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser. The reconstituted botulinum toxin composition is then applied to the skin of the subject in the affected areas and in the areas in which the Spongilla composition was applied taking care to avoid mucous membranes (e.g., eyes, mouth, and nostrils). The botulinum toxin composition is allowed to dry on the application area and is then cleaned using non-comedogenic wipes or water and a non-comedogenic cleanser.

Weeks 2 through 6: Three percent (3%) hydrogen peroxide USP (6 mL) are added to 3 grams (dry weight) of Spongilla powder composition and the resulting mixture is stirred thoroughly mixed. The Spongilla mixture is applied to the affected area of the subject's skin taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser.

Maintenance Regimen: After the 6-week treatment regimen is complete, the subject can apply the composition comprising Spongilla and/or Spongilla and 3% hydrogen peroxide to the affected areas monthly. The application of the composition comprising botulinum toxin may be repeated as indicated by the symptoms of the subject, but no sooner than 3 months since the immediately prior treatment using the composition comprising the botulinum toxin.

### Example 6

Week 1: The subject's skin is washed and dried with a non-comedogenic cleanser. A container comprising botulinum toxin (botulinum toxin type A, botulinum toxin type B, botulinum toxin type C1, botulinum toxin type C2, botulinum toxin type D, botulinum toxin type E, botulinum toxin type F and botulinum toxin type G, onabotulinumtoxinA, abobotulinumtoxinA, incobotulinumtoxinA, prabotulinumtoxinA, daxibotulinumtoxinA, rimabotulinumtoxinB, EB-001A, or EB-001T) is reconstituted with 10 mL of 0.9% sterile saline and the reconstituted solution is set aside. The reconstituted botulinum toxin composition is then applied to the skin of the subject in the affected areas, taking care to avoid mucous membranes (e.g., eyes, mouth, and nostrils). A composition comprising Spongilla powder is then applied to the affected area of the subject's skin taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser.

Weeks 2 through 6: Three percent (3%) hydrogen peroxide USP (6 mL) are added to 3 grams (dry weight) of Spongilla powder composition and the resulting mixture is stirred thoroughly mixed. The Spongilla mixture is applied to the affected area of the subject's skin, taking care to avoid mucous membranes (e.g., eyes, mouth, and nostrils). Additional Spongilla materials may be applied to areas on the subject's skin where lesions and hyperpigmentation are present. The Spongilla composition is allowed to dry on subject's skin to which it was applied and is then removed using non-comedogenic wipes or water and a non-comedogenic cleanser.

Maintenance Regimen: After the 6-week treatment regimen is complete, the subject can apply the composition comprising Spongilla and/or Spongilla and 3% hydrogen peroxide to the affected areas monthly. The application of the composition comprising botulinum toxin may be repeated as indicated by the symptoms of the subject, but no sooner than 3 months since the immediately prior treatment using the composition comprising the botulinum toxin.

### Example 7: Treatment of Subjects Having Moderate to Severe Facial Acne Vulgaris with a Combination of Spongilla lacustris and OnabotulinumtoxinA

The objective of the study is to evaluate the tolerability, safety, and efficacy of powdered Spongilla lacustris administered with onabotulinumtoxinA (BOTOX®) 100U following 6 weeks of topical administration (comprising one topical treatment using onabotulinumtoxinA and once-weekly topical treatment of Spongilla lacustris in male and female subjects with moderate to severe facial acne vulgaris.

The study is an open-label, parallel-group study of approximately 12 weeks duration (day 1 treatment to day 85 study exit). The study groups will receive treatment with a combination of powdered Spongilla lacustris, that is mixed with 3% H₂O₂ or water prior to administration, and onabotulinumtoxinA (BOTOX®) 100U. Powdered Spongilla lacustris is administered to the entire face of the subject for once weekly for 6 weeks and onabotulinumtoxinA (BOTOX®) 100U is administered topically to the face of the subject on study day 1. On day 1, subjects are randomly assigned to 1 of 6 treatment groups in a 1:1:1:1:1:1 ratio to receive one of six regimens as summarized in Table 1.

TABLE 1
Treatment Allocation

| Treatment | |
|---|---|
| Regimen | Study Treatment |
| 1 | Topical application of a first composition |
| | comprising Spongilla lacustris powder mixed with |
| | 3% H₂O₂, followed by topical application of a |
| | second composition comprising onabotulinumtoxinA |
| | (BOTOX ®) 100 U |
| 2 | Topical application of a first composition |
| | comprising onabotulinumtoxinA (BOTOX ®) |
| | 100 U, followed by topical application of a second |
| | composition comprising Spongilla lacustris |
| | powder mixed with 3% H₂O₂ |
| 3 | Topical application of a single composition |
| | comprising onabotulinumtoxinA (BOTOX ®) 100 U |
| | and Spongilla lacustris powder mixed with 3% |
| | H₂O₂ |
| 4 | Topical application of a first composition |
| | comprising Spongilla lacustris powder mixed with |
| | water, followed by topical application of a second |
| | composition comprising onabotulinumtoxinA |
| | (BOTOX ®) 100 U |
| 5 | Topical application of a first composition |
| | comprising onabotulinumtoxinA (BOTOX ®) |
| | 100 U, followed by topical application of a |
| | second composition comprising Spongilla |
| | lacustris powder mixed with water |
| 6 | Topical application of a single composition |
| | comprising onabotulinumtoxinA (BOTOX ®) |
| | 100 U and Spongilla lacustris powder mixed |
| | with water |

### Study Population

Male and female subjects 12 years or older with moderate to severe acne vulgaris with facial involvement who are in otherwise good health are enrolled.

### Inclusion Criteria

Each subject has moderate to severe acne vulgaris defined as meeting all of the following criteria: (a) a minimum of 20 but not more than 150 inflammatory lesions (papules and pustules) on the face; (b) a minimum of 20 but not more than 300 noninflammatory lesions (open comedones and closed comedones) on the face; (c) not more than 2 nodules on the face above the mandibular line; and (d) an investigator's Global Assessment (IGA) score of 3 or 4 as assessed by the investigator (the area considered for the IGA must be confined to the face)

### Exclusion Criteria:

Subjects are excluded from the study if they have a known allergy or sensitivity to any botulinum toxin preparation, or if they have any medical condition that may put them at increased risk with exposure to onabotulinumtoxinA (BOTOX®) 100U, including diagnosed myasthenia gravis, Eaton-Lambert syndrome, or amyotrophic lateral sclerosis

### Measurement of Efficacy:

Efficacy is measured by lesion counts (inflammatory and noninflammatory) on the face of each subject and by use of an Investigator's Global Assessment using the scores in Table 2, below. Three analysis populations will be used as follows: (a) the intent-to-treat (ITT) population will consist of all randomized subjects; (b) the per protocol (PP) population will consist of all randomized subjects with no significant protocol violations during the study that would affect the efficacy analyses (the PP population will be determined prior to database lock); and (c) the safety population will consist of all subjects who receive at least 1 dose of study medication in the study.

All efficacy analyses will be performed using the ITT and PP population. For lesion counts, absolute and percent change from baseline in lesion counts will be summarized using descriptive statistics. A frequency distribution will be used to analyze the proportion of subjects with a complete or partial response.

TABLE 2
Investigator's Global Assessment (IGA)

| Score | Grade | Description |
|---|---|---|
| 0 | None | No evidence of facial acne vulgaris |
| 1 | Minimal | Few noninflammatory lesions (comedones) are present; |
| | | a few inflammatory lesions(papules/pustules) may be |
| | | present; no nodulo-cystic lesions are allowed) |
| 2 | Mild | Several to many noninflammatory lesions (comedones) |
| | | are present; a few inflammatory lesions |
| | | (papules/pustules) are present; no nodulo-cystic lesions |
| | | are allowed |
| 3 | Moderate | Many noninflammatory (comedones) and |
| | | inflammatory lesions (papules/pustules) are present; no |
| | | nodulo-cystic lesions are allowed |
| 4 | Severe | Significant degree of inflammatory disease; |
| | | papules/pustules are a predominant feature; a few |
| | | nodulo-cystic lesions may be present; comedones may |
| | | be present |

The safety of the treatment regimens is evaluated by measuring the number of any events that are determined to be treatment-related adverse events, directed physical examination, measurement of vital signs (including height, weight, body temperature, pulse rate, respiratory rate, and blood pressure), and local (dermal) tolerability (as measured by the investigator's assessment of the dryness, scaling and/or erythema of a subject's skin in the treatment area). All adverse events will be coded from the verbatim text to the lower level term and mapped to preferred term (PT) and primary system organ class (SOC) using the Medical Dictionary for Regulatory Activities.

Data will be summarized with descriptive statistics with 95% confidence intervals, frequency tables, and data listings.

### Sample Size Calculation:

Approximately 60 subjects will be entered into the study. When the sample size is 10 per group, a two-sided 95.0% confidence interval for a single proportion using the binomial approximation will extend 0.262 to 0.878 for an expected proportion of 0.600 of IGA responders. When the sample size is 10 per group, a two-sided 95.0% confidence interval for the mean change from baseline lesion counts using the normal approximation will extend −7.85 to −22.15 assuming a mean change from baseline of −15 and a standard deviation of 10.

The application of Spongilla lacustris and onabotulinumtoxinA (BOTOX®) 100U has an acceptable response rate and decreases the total number of lesions in the treatment area in subjects with acne vulgaris after 6 weeks of topical administration (one topical treatment of onabotulinumtoxinA (BOTOX®) 100U and once-weekly topical treatment with Spongilla), measured by both investigator's global assessment (IGA), and total lesion counts (nodules, cysts, inflammatory and non-inflammatory).

### Example 8: Preparation of Spongilla Materials

Spongilla raw material was treated with heat or gamma irradiation to reduce bioburden to acceptable levels. The resulting materials were milled and sieved to obtain a material having a particle size of no larger than 200 μm. The sized material was dried using a low temperature tray dryer at a temperature of about 40° C. to obtain a target moisture content of less than 1%. A representative batch of materials was irradiated as follows using Nordion Cobalt-60 Batch JS 8900 Irradiator #139, ON-STD; processing start time=10:57:38 am; processing end time: 01:31:14 pm; minimum specified dose (kGy): 25.0; maximum specified dose (kGy): 32.5; minimum delivered dose (kGy): 26.2; maximum delivered dose (kGy): 31.2. The resulting material was filled and sealed into high-density polyethylene jars that were packaged into foil laminate pouches containing desiccant packets. The packaged product may be further gamma irradiated if the material does not otherwise meet the microbial limits set forth in USP <1111>. The resulting materials were stored at a temperature of below 30° C. and were protected from light and moisture prior to use.

### Example 9: Treatment of Subjects Having Hyperhidrosis with a Combination of Spongilla lacustris and onabotulinumtoxinA

The objective of the study was to evaluate the tolerability, safety, and efficacy of (a) Spongilla powder mixed with 3% hydrogen peroxide USP, or (b) Spongilla powder, mixed with 0.9% sodium chloride USP, followed by topical administration of BOTOX® (onabotulinumtoxinA) Purified Neurotoxin complex to the axilla in subjects with primary axillary hyperhidrosis. The study was structured as a single center, two arm, open label, study of approximately 4 weeks (day 1 treatment to day 29 study exit).

To be enrolled in the study, each subject had diagnosis of primary, axillary hyperhidrosis within 6 months of baseline (study day 1) and a HDSS score of 3 or 4 (based on both axillae) using the criteria in Table 3. The subject must also have had sweat production of at least 50 mg over about 5 minutes in each axilla assessed gravimetrically.

TABLE 3
HYPERHIDROSIS DISEASE SEVERITY SCALE (HDSS)

| Score | Description |
|---|---|
| 1 | My underarm sweating is never noticeable and never interferes |
| | with my daily activities |
| 2 | My underarm sweating is tolerable and sometimes interferes with |
| | my daily activities |
| 3 | My underarm sweating is barely tolerable and frequently interferes |
| | with my daily activities |
| 4 | My underarm sweating is intolerable and always interferes with |
| | my daily activities |

All subjects received active therapy in both right and left axillae on day one of the study. Each subject received Spongilla powder mixed with 3% hydrogen peroxide USP in one axilla and Spongilla powder mixed with 0.9% sodium chloride for injection USP in the contralateral axilla. The investigational products were as set forth in Table 4.

TABLE 4
Description of Investigational Products

| | |
|---|---|
| IP Name: | Spongilla topical powder plus hydrogen peroxide |
| Active Ingredient: | 2 gm of Spongilla powder |
| Other Ingredients: | 6 mL of 3% hydrogen peroxide USP |
| IP Name: | Spongilla topical powder plus sodium chloride solution |
| Active Ingredient: | 2 gm of Spongilla powder |
| Other Ingredients: | 6 mL of 0.9% sodium chloride for Injection USP |
| IP Name: | BOTOX ® (Botulinum Toxin Type A) Purified |
| | Neurotoxin Complex |
| Active Ingredient: | 100 units (U) of Clostridium botulinum toxin Type A |
| Other Ingredients: | 0.5 mg albumin (human), and 0.9 mg sodium chloride |

The choice of diluent (3% hydrogen peroxide USP or 0.9% sodium chloride) was randomly allocated to right and left axillae for each subject. After approximately 10 minutes, the composition containing Spongilla was removed from the subject's skin with Cetaphil® wipes and onabotulinumtoxinA 100U was massaged into the axilla. Reconstituted onabotulinumtoxinA 100U was applied to each axilla and massaged into the skin of the axilla. The onabotulinumtoxinA composition was allowed to remain on the skin for approximately 15 minutes, then the subject's skin was cleaned with a pre-moistened cleaning wipe (e.g., Cetaphil® wipe) and then the area was gently dried. Treatments were applied once, on study day 1.

Gravimetric sweat production in each subject was measured (a) prior to day one of the study; (b) on study day one in which the treatment is applied to the subject; and (c) on study day 29±3. The measurement of gravimetric sweat production was performed on each axilla at the Screening, Day 1, and 29/early exit visits. Gravimetric assessment were conducted after about 15 minutes at rest in a sitting position. All tests were performed in the same room at a room temperature of about 25° C. The axillae were thoroughly cleaned with an absorbent paper before gravimetry. A 90-mm diameter round filter paper was weighed with a microbalance and the weight recorded. The filter paper was placed under the axilla and a plastic bag was placed under the filter paper, to avoid evaporation of sweat. After about 5 minutes, the round filter paper was reweighed, and the difference between the 2 weights was taken as sweat production in milligrams over about 5 minutes. Each subject's overall severity of hyperhidrosis was also evaluated by a 4-point HDSS assessment that is self-assessment by the subject and was completed (a) at screening; (b) on study day one; (c) and on study day 29 t 3 or earlier if the subject did not complete the 29-day study.

Treatment efficacy was measured as the percentage of subjects with a ≥2-grade improvement in the Hyperhidrosis Disease Severity Scale (HDSS) from baseline 4 weeks following treatment. Another measure of treatment efficacy was the percentage of subjects having ≥50% reduction in gravimetrically measured sweat production from baseline in the 4 weeks following treatment. Another measure of treatment efficacy was the mean absolute change from baseline in gravimetrically-measured sweat production in subjects in the 4 weeks following treatment. Three analysis populations were used as follows: (1) the intent-to-treat (ITT) population will consist of all enrolled subjects; (2) the per protocol (PP) population will consist of all enrolled subjects with no significant protocol violations during the study that would affect the efficacy analyses. The PP population was determined prior to database lock; and (3) the safety population consisted of all subjects who receive at least 1 dose of study medication in the study. Efficacy Analysis: All efficacy analyses were performed using the ITT and PP population. For continuous variables, (e.g., change from baseline in gravimetric sweat production) data was summarized using descriptive statistics by treatment group with 95% confidence intervals. For categorical variables, a frequency distribution was used to analyze the proportion of subjects with a response (e.g., % of subjects with ≥50% reduction in gravimetric sweat production).

TABLE 5
Summary of Sweat Gravimetric Sweat Production and
Hyperhidrosis Disease Severity Scale (HDSS)

| | Day 1 | | Day 2 | | | | | |
|---|---|---|---|---|---|---|---|---|
| | Gravimetric Test | | Gravimetric Test | | Day 1 HDSS | | Day 29 HDSS | |
| Subject | Left | Right | Left | Right | Left | Right | Left | Right |
| No. | axilla | Axilla | axilla | Axilla | axilla | Axilla | axilla | Axilla |
| 001-001 | 83 mg | 110 mg | 5 mg | 1 mg | 3 | 3 | 3 | 3 |
| | (0.9% | (H₂O₂) | (0.9% | (H₂O₂) | | | | |
| | saline) | | saline) | | | | | |
| 001-006 | 90 mg | 60 mg | 34 mg | 28 mg | 4 | 3 | 2 | 2 |
| | (H₂O₂) | (0.9% | (H₂O₂) | (0.9% | | | | |
| | | saline) | | saline) | | | | |
| 001-008 | 275 mg | 310 mg | 183 mg | 284 mg | 4 | 4 | 4 | 4 |
| | (H₂O₂) | (0.9% | (H₂O₂) | (0.9% | | | | |
| | | saline) | | saline) | | | | |
| 001-011 | 55 mg | 73 mg | 4 mg | 4 mg | 4 | 4 | 4 | 4 |
| | (0.9% | (H₂O₂) | (0.9% | (H₂O₂) | | | | |
| | saline) | | saline) | | | | | |
| 001-012 | 230 mg | 165 mg | 15.7 mg | 36.6 mg | 3 | 3 | 3 | 3 |
| | (H₂O₂) | (0.9% | (H₂O₂) | (0.9% | | | | |
| | | saline) | | saline) | | | | |
| 001-013 | 122 mg | 151 mg | 5.7 mg | 9.1 mg | 3 | 3 | 2 | 2 |
| | (H₂O₂) | (0.9% | (H₂O₂) | (0.9% | | | | |
| | | saline) | | saline) | | | | |
| 001-014 | 100 mg | 103 mg | 41.1 mg | 53 mg | 4 | 4 | 3 | 3 |
| | (0.9% | (H₂O₂) | (0.9% | (H₂O₂) | | | | |
| | saline) | | saline) | | | | | |
| 001-016 | 231 mg | 280 mg | 3 mg | 11 mg | 3 | 3 | 3 | 3 |
| | (0.9% | (H₂O₂) | (0.9% | (H₂O₂) | | | | |
| | saline) | | saline) | | | | | |
| 001-025 | 274 mg | 208 mg | 22 mg | 10 mg | 3 | 3 | 3 | 3 |
| | (0.9% | (H₂O₂) | (0.9% | (H₂O₂) | | | | |
| | saline) | | saline) | | | | | |
| 001-027 | 61 mg | 133 mg | 40 mg | 31 mg | 3 | 3 | 3 | 3 |
| | (H₂O₂) | (0.9% | (H₂O₂) | (0.9% | | | | |
| | | saline) | | saline) | | | | |

TABLE 6
Summary of Response Rate in Subjects
(0.9% saline and H₂O₂ arms combined)

| | Total No. Axilla = 20 (10 subjects each | | |
|---|---|---|---|
| | contributed 2 axilla that received treatment) | | |
| | Response Rate | Z-score | p-value |
| Decrease in | 80% | 2.26 | 0.024 |
| gravimetric sweat | | | |
| production ≥ 50% at | | | |
| Week 4 | | | |
| Gravimetric sweat | 85% | 2.58 | 0.009 |
| production < 50 mg at | | | |
| Week 4 | | | |

In Table 6, the Z-score is from a one sample test for a single proportion. Assumption for the analysis is that the observed proportion is not different from 40% (Placebo Response Rate) (Fleiss, D. B. 1981. Statistical Methods for Rates and Proportions, 2nd Ed. Wiley and Sons, p. 13-14).

TABLE 7
Mean Change in Gravimetric Sweat Production

| | | Hydrogen peroxide | |
|---|---|---|---|
| | Saline Arm | arm | p-value |
| No. of Subjects | 10 | 10 | |
| Mean change in | −109.82 | −119.46 | 0.791 |
| gravimetric sweat | | | |
| production at week 4 | | | |
| Percent change in | −0.746 | −0.752 | 0.965 |
| gravimetric sweat | | | |
| production at week 4 | | | |

TABLE 8
Summary of Gravimetric Sweat Production in Subjects
(0.9% saline and H₂O₂ arms combined)

| | Gravimetric Measurements (mg sweat) by Time | | | |
|---|---|---|---|---|
| | Total No. Axilla = | | | |
| | 20 (10 subjects each | | | |
| | contributed 2 axilla | | | |
| | that received | | | |
| | treatment) | | t-score | p-value |
| Baseline sweat | 155.7 | mg | | |
| production (mean) | | | | |
| Sweat production in | 41.0 | mg | | |
| week 4 following | | | | |
| treatment (mean) | | | | |
| Mean change in | −114.64 | mg | 2.44 | 0.024 |
| sweat production | | | | |
| from baseline in | | | | |
| week 4 following | | | | |
| treatment (mg) | | | | |
| Mean Percent | −75% | | 8.93 | <0.001 |
| Change from | | | | |
| Baseline | | | | |

In table 8, the t-score of 2.44 is from a one sample test for a single mean and assumes for the analysis is that the observed mean change is not different from 72 mg. In table 8, the t-score of 8.93 is from a one sample test for a single mean and assumes for the analysis is that the observed mean percent change is not different from 21%.

TABLE 9
Treatment Emergent Adverse Events

| | No. Subjects (% out of 10 |
|---|---|
| Adverse Event | subjects in study) |
| Subjects reporting at least one AE | 2 (20%) |
| Skin and subcutaneous tissue disorders | 1 (10%) |
| Irritant Dermatitis | 1 (10%) |
| Renal and Urinary Disorders | 1 (10%) |
| Urinary Tract Infection | 1 (10%) |

Subject 01-016 experienced dermatitis of the right axilla 2 days post treatment with DMT410 plus hydrogen peroxide. Subject applied ice and aloe vera gel to the affected are and the event resolved completely within 24 hours.

1. A method of treating hyperhidrosis in a subject, comprising applying to the skin of the subject a first composition comprising Spongilla, and a second composition comprising one or more botulinum toxins.

2. The method according to claim 1, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G.

3. The method according to claim 2, wherein the second composition comprises one or more botulinum toxin type selected from botulinum toxin type A.

4. The method according to claim 3, wherein the botulinum toxin type A is onabotulinumtoxinA.

5. The method according to claim 1, wherein the subject experiences at least a two-grade improvement in the HDSS score following treatment compared to the subject's HDSS score prior to treatment.

6. The method according to claim 1, wherein the subject experiences a greater than 10% reduction in gravimetrically-measured sweat production following treatment compared to the subject's gravimetrically-measured sweat production prior to treatment.

7. The method according to claim 1, wherein the subject experiences at least a 4-point improvement from baseline in the weekly mean Axillary Sweating Daily Diary (ASDD) item #2 score compared to the subject's ASDD score prior to treatment.

8. The method according to claim 1, wherein the subject is treated once per month, or once every two months, or once every three months, or once every four months, or once every five months, or once every six months, or once every seven months, or once every eight months, or once every nine months, or once every 10 months, or once every 11 months, or once every 12 months.

9. The method according to claim 1, wherein the subject is treated once per week.

10. The method according to claim 1, wherein the subject suffers from primary axillary hyperhidrosis.

11. The method according to claim 1, wherein the first composition comprises from about 0.25 grams to about 10 grams Spongilla.

12. The method according to claim 1, wherein the first composition comprises about 2 grams of Spongilla and about 6 mL of 3% hydrogen peroxide or about 6 mL of saline.

13. A composition comprising a first composition and a second composition for use in the treatment of hyperhidrosis in a subject, wherein (a) the first composition comprises Spongilla; and (b) the second composition comprises one or more botulinum toxins.

14. The composition according to claim 13, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G.

15. The composition according to claim 14, wherein the second composition comprises one or more botulinum toxin type selected from botulinum toxin type A.

16. The composition according to claim 15, wherein the botulinum toxin type A is onabotulinumtoxinA.

17. A kit, comprising a first composition and a second composition, wherein:
(a) the first composition comprises Spongilla; and
(b) the second composition comprises one or more botulinum toxins.

18. The kit according to claim 17 for use in the treatment of a subject having hyperhidrosis.

19. The kit according to claim 17, wherein the second composition comprises one or more botulinum toxin type selected from botulinum toxin type A, botulinum toxin type B, botulinum toxin type C1, botulinum toxin type C2, botulinum toxin type D, botulinum toxin type E, botulinum toxin type F and botulinum toxin type G.

20. The kit according to claim 1, wherein the botulinum toxin is onabotulinumtoxinA.
